(19) United States
(12) Patent Application Publication
(10) Pub. No.: US 2011/0023162 A1
(43) Pub. Date: Jan. 27, 2011

(54) GENETICALLY MODIFIED CHRYSANTHEMUMS

(75) Inventor: Filippa Brugliera, Victoria (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: International Flower Developments Pty. Ltd., Victoria (AU)

(21) Appl. No.: 12/743,165

(22) PCT Filed: Nov. 14, 2008
(86) PCT No.: PCT/AU2008/001694

§ 371 Date: Sep. 28, 2010

Related U.S. Application Data
(60) Provisional application No. 60/988,331, filed on Nov. 15, 2007.

(51) Int. Cl. A01H 1/04; A01H 5/00; A01H 5/02; C12N 5/10; C12N 15/82

(52) U.S. Cl. 800/260; 800/323.2; 435/419; 800/282

(57) ABSTRACT

The present invention relates to genetically modified chrysanthemum plants expressing altered inflorescence. The chrysanthemum flavonoid pathway is manipulated to produce plants with blue or violet inflorescence. Blue delphinidin pigments are produced by the expression of flavonoid 3′5′ hydroxylase and (optionally) the suppression of flavonoid 3′ hydroxylase activity.

### FILING DATA

This application is associated with and claims priority from U.S. Provisional Patent Application No. 60/988,331, filed on 15 Nov. 2007, the entire contents of which, are incorporated herein by reference.

### FIELD

The present invention relates generally to the field of genetically modified plants. More particularly, the present invention is directed to genetically modified ornamental flowering plants expressing selected color phenotypes in desired parts of the plants. Even more particularly, the present invention provides genetically modified chrysanthemum plants expressing altered or desired inflorescence.

### BACKGROUND

Bibliographic details of references provided in the subject specification are listed at the end of the specification.

Reference to any prior art is not, and should not be taken as an acknowledgment or any form of suggestion that this prior art forms part of the common general knowledge in any country.

The flower or ornamental plant industry strives to develop new and different varieties of flowers and/or plants. An effective way to create such novel varieties is through the manipulation of flower color. Classical breeding techniques have been used with some success to produce a wide range of colors for almost all of the commercial varieties of flowers and/or plants available today. This approach has been limited, however, by the constraints of a particular species' gene pool and for this reason it is rare for a single species to have the full spectrum of colored varieties. For example, the development of novel colored varieties of plants or plant parts such as flowers, foliage and stems would offer a significant opportunity in both the cut flower and ornamental markets. In the flower or ornamental plant industry, the development of novel colored varieties of chrysanthemum is of particular interest. This includes not only different colored flowers but also anthers and styles.

Flower color is predominantly due to three types of pigment: flavonoids, carotenoids and betalains. Of the three, the flavonoids are the most common and contribute to a range of colors from yellow to red to blue. The flavonoid molecules that make the major contribution to flower color are the anthocyanins, which are glycosylated derivatives of cyanidin and its methylated derivative peonidin, delphinidin and its methylated derivatives petunidin and malvidin and pelargonidin. Anthocyanins are localized in the vacuole of the epidermal cells of petals or the vacuole of the sub epidermal cells of leaves.

The flavonoid pigments are secondary metabolites of the phenylpropanoid pathway. The biosynthetic pathway for the flavonoid pigments (flavonoid pathway) is well established, (Holton and Cornish, Plant Cell 7:1071-1083, 1995; Mol et al, Trends Plant Sci. 3:212-217, 1998; Winkel-Shirley, Plant Physiol. 126:485-493, 2001a; and Winkel-Shirley, Plant Physiol. 127:1399-1404, 2001b, Tanaka and Mason, In Plant Genetic Engineering, Singh and Jaiwal (eds) SciTech Publishing Llc., USA, 1:361-385, 2003, Tanaka et al, Plant Cell, Tissue and Organ Culture 80:1-24, 2005, Tanaka and Brugliera, In Flowering and Its Manipulation, Annual Plant Reviews Ainsworth (ed), Blackwell Publishing, UK, 20:201-239, 2006) and is shown in FIG. 1. Three reactions and enzymes are involved in the conversion of phenylalanine to p-coumaroyl-CoA, one of the first key substrates in the flavonoid pathway. The enzymes are phenylalanine ammonia-lyase (PAL), cinnamate 4-hydroxylase (C4H) and 4-coumarate: CoA ligase (4CL). The first committed step in the pathway involves the condensation of three molecules of malonyl-CoA (provided by the action of acetyl CoA carboxylase (ACC) on acetyl CoA and CO₂) with one molecule of p-coumaroyl-CoA. This reaction is catalyzed by the enzyme chalcone synthase (CHS). The product of this reaction, 2′,4,4′,6′, tetrahydroxy-chalcone, is normally rapidly isomerized by the enzyme chalcone flavanone isomerase (CHI) to produce naringenin. Naringenin is subsequently hydroxylated at the 3 position of the central ring by flavanone 3-hydroxylase (F3H) to produce dihydrokaempferol (DHK).

The pattern of hydroxylation of the B-ring of dDHK plays a key role in determining petal color. The B-ring can be hydroxylated at either the 3′, or both the 3′ and 5′ positions, to produce dihydroquercetin (DHQ) or dihydromyricetin (DHM), respectively. Two key enzymes involved in this part of the pathway are flavonoid 3′-hydroxylase (F3′H) and flavonoid 3′,5′-hydroxylase (F3′5′H), both members of the cytochrome P450 class of enzymes. F3′H is involved in the generation of cyanidin-based pigments which, in many plant species, contribute to red and pink flower color.

Nucleotide sequences encoding F3′5′Hs have been cloned (see International Patent Application No. PCT/AU92/00334 and Holton et al, Nature, 366:276-279, 1993 and International Patent Application No. PCT/AU03/01111). These sequences were efficient in modulating 3′, 5′ hydroxylation of flavonoids in petunia (see International Patent Application No. PCT/AU92/00334 incorporated herein by reference and Holton et al, 1993, supra), tobacco (see International Patent Application No. PCT/AU92/00334 incorporated herein by reference), carnations (see International Patent Application No. PCT/AU96/00296 incorporated herein by reference) and roses (see International Patent Application No. PCT/AU03/01111 incorporated herein by reference).

The next step in the pathway, leading to the production of the colored anthocyanins from the dihydroflavonols (DHK, DHQ, DHM), involves dihydroflavonol-4-reductase (DFR) leading to the production of the leucoanthocyanidins. The leucoanthocyanidins are subsequently converted to the anthocyanidins, pelargonidin, cyanidin and delphinidin. These flavonoid molecules are unstable under normal physiological conditions and glycosylation at the 3-position, through the action of glycosyltransferases, stabilizes the anthocyanidin molecule thus allowing accumulation of the anthocyanins. In general, the glycosyltransferases transfer the sugar moieties from UDP sugars to the flavonoid molecules and show high specificities for the position of glycosylation and relatively low specificities for the acceptor substrates (Seitz and Hinderer, Anthocyanins. In: Cell Culture and Somatic Cell Genetics of Plants. Constabel and Vasil (eds.). Academic Press. New York, USA, 5:49-76, 1988). Anthocyanins can occur as 3-monosides, 3-biosides and 3-triosides as well as 3,5-diglycosides and 3,7-diglycosides associated with the sugars glucose, galactose, rhamnose, arabinose and xylose (Strack and Wray, In: The Flavonoids—Advances in Research since 1986. Harborne, J. B. (ed), Chapman and Hall, London, UK, 1-22, 1993).

Chrysanthemums are a highly evolved flowering plant. A member of the Asteraceae family, species of Chrysanthemum are herbaceous perennial plants growing to 50-150 cm tall, with deeply lobed leaves and large flower heads, white, yellow or pink in the wild species. The flowers occur in various forms and can be daisy-like, decorative, pompons or buttons. The National Chrysanthemum Society (USA) divides chrysanthemums into 13 classes based on flower shape. The classes are respectively irregular incurve, reflex, regular incurve, decorative, intermediate incurve, pompon, single and semi-double, anemone, spoon, quill, spider, brush or thistle and unclassified.

The anthocyanins found in chrysanthemums are generally based on cyanidin. Delphinidin-based pigments are not present due to the lack of a F3′5′H activity and pelargonidin-based pigments are rarely found. It has been suggested that the absence of pelargonidin-based pigments in chrysanthemum is due to the presence of a F3′H activity rather than the DFR specificity. For example, when chrysanthemum petals were fed with a cytochrome P450 inhibitor, pelargonidin-based pigments were detected (Schwinn et al, Phytochemistry, 35:145-150, 1993).

Whilst classical breeding practices have produced hybrid chrysanthemums, genetic engineering has failed to produce a violet/purple to blue color in chrysanthemum flowers. There is a need to develop ornamental flowering plants and in particular chrysanthemum plants with altered inflorescence.

### SUMMARY

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO). The SEQ ID NOs correspond numerically to the sequence identifiers <400>1 (SEQ ID NO:1), <400>2 (SEQ ID NO:2), etc.

A summary of sequence identifiers used throughout the subject specification is provided in Table 1.

The present invention provides genetically modified ornamental flowering plants and in particular genetically modified chrysanthemums exhibiting altered including selected or desired inflorescence. Even more particularly, the altered inflorescence is a color in the range of red-purple to blue in chrysanthemum tissue or organelles including flowers, petals, anthers and styles. In one embodiment, the color is determined using the Royal Horticultural Society (RHS) color chart where colors are arranged in order of the fully saturated colors with the less saturated and less bright colors alongside. The color groups proceed through the spectrum and the colors referred to in this application are generally in the red-purple (RHSCC 58-74), purple (RHSCC 75-79), purple-violet (RHSCC 81-82), violet (RHSCC 83-88), violet-blue (RHSCC 89-98), blue (RHSCC 99-110) groups contained in Fan 2. Colors are selected from the range including 75C, 75D, 76A, 76B, 76C, 77C, 77D, 78D, N80D, N81B, 84C and colors in between or proximal thereto. In particular, genetically modified chrysanthemums are provided which produce delphinidin and which are purple/violet to blue in color. Non-genetically modified chrysanthemums do not produce delphinidin-based flavonoid molecules due to an absence of F3′5′H activity. Color change is facilitated by the presence of delphinidin. Conveniently, delphinidin can be detected using TLC or HPLC.

TABLE 1
Summary of sequence identifiers

| SEQ ID | | | TYPE | |
|---|---|---|---|---|
| NO: | NAME | SPECIES | OF SEQ | DESCRIPTION |
| 1 | BPF3′5′H#18.nt | Viola sp | nucleotide¹ | F3′5′H cDNA |
| 2 | BPF3′5′H#18.aa | Viola sp | amino acid | deduced F3′5′H amino |
| | | | | acid sequence |
| 3 | BPF3′5′H#40.nt | Viola sp | nucleotide¹ | F3′5′H cDNA |
| 4 | BPF3′5′H#40.aa | Viola sp | amino acid | deduced F3′5′H amino |
| | | | | acid sequence |
| 5 | SalF3′5′H#47.nt | Salvia sp | nucleotide¹ | F3′5′H cDNA |
| 6 | SalF3′5′H#47.aa | Salvia sp | amino acid | deduced F3′5′H amino |
| | | | | acid sequence |
| 7 | PetHf1.nt | Petunia hybrida | nucleotide | F3′5′H cDNA |
| 8 | PetHf1.aa | Petunia hybrida | amino acid | deduced F3′5′H amino |
| | | | | acid sequence |
| 9 | RoseCHS 5′ | Rosa hybrida | nucleotide | promoter sequence |
| 10 | carnANS 5′ | Dianthus | nucleotide | promoter sequence |
| | | caryophyllus | | |
| 11 | carnANS 3′ | Dianthus | nucleotide | terminator sequence |
| | | caryophyllus | | |
| 12 | Cineraria gF3′5′H | Cineraria sp | nucleotide | F3′5′H genomic clone |
| 13 | Cineraria gF3′5′H | Cineraria sp | amino acid | deduced F3′5′H amino |
| | | | | acid sequence |
| 14 | Cineraria F3′5′H 5′ | Cineraria sp | nucleotide | promoter sequence |
| 15 | Cineraria F3′5′H 3′ | Cineraria sp | nucleotide | terminator sequence |
| 16 | R. rugosa DFR 5′ | Rosa rugosa | nucleotide | promoter sequence |
| 17 | R. rugosa F3H 5′ | Rosa rugosa | nucleotide | promoter sequence |
| 18 | BPF3′5′H#40 5′ | Viola sp | nucleotide | promoter sequence |
| 19 | Hf1-5 | P. hybrida | nucleotide | primer |
| 20 | Hf1-3 | P. hybrida | nucleotide | primer |
| 21 | CHSA+34 | P. hybrida | nucleotide | primer |
| 22 | CHSA−782 | P. hybrida | nucleotide | primer |
| 23 | C1 | | nucleotide | primer |
| 24 | BP40-i5 | Viola sp. | nucleotide | primer |
| 25 | C2 | | nucleotide | primer |
| 26 | BP-i7 | Viola sp. | nucleotide | primer |
| 27 | BP40 pro-F | Viola sp. | nucleotide | primer |
| 28 | BP40 pro-HindIII-F | Viola sp. | nucleotide | primer |
| 29 | BP40 pro-NheI-R | Viola sp. | nucleotide | primer |
| 30 | BP40 pro-NheI-F | Viola sp. | nucleotide | primer |
| 31 | BP40 pro-BamHI-R | Viola sp. | nucleotide | primer |
| 32 | DFRproHindIIIF | R. rugosa | nucleotide | primer |
| 33 | DFRproHneIR | R. rugosa | nucleotide | primer |
| 34 | DFRproNheIF | R. rugosa | nucleotide | primer |
| 35 | DFRproBamHI-R | R. rugosa | nucleotide | primer |
| 36 | RrF3H-F | R. rugosa | nucleotide | primer |
| 37 | RrF3H | R. rugosa | nucleotide | primer |
| 38 | DFRint F | Petunia | nucleotide | primer |
| 39 | DFRint R | Petunia | nucleotide | primer |
| 40 | Chrys Red F | Chrysanthemum | nucleotide | primer |
| 41 | Chrys Red R | Chrysanthemum | nucleotide | primer |
| 42 | 3369Xba.F | Chrysanthemum | nucleotide | primer |
| 43 | 3369Xba.R | Chrysanthemum | nucleotide | primer |
| 44 | chrysF3′H.nt | Chrysanthemum | nucleotide | F3′H cDNA clone |
| 45 | chrysF3′H.aa | Chrysanthemum | amino acid | deduced F3′H amino |
| | | | | acid sequence |

¹See PCT/AU03/01111
²See Holton et al, 1993, supra and PCT/AU92/00334

The present invention provides genetically modified ornamental flowering plants and in particular genetically modified chrysanthemums exhibiting altered including selected or desired inflorescence. Even more particularly, the altered inflorescence is a color in the range of red-purple to blue in chrysanthemum tissue or organelles including flowers, petals, anthers and styles. Color is subjective to determine. In one embodiment, the color is determined using the Royal Horticultural Society (RHS) color chart where colors are arranged in order of the fully saturated colors with the less saturated and less bright colors alongside. The color groups proceed through the spectrum and the colors referred to in this application are generally in the red-purple (RHSCC 58-74), purple (RHSCC 75-79), purple-violet (RHSCC 81-82), violet (RHSCC 83-88), violet-blue (RHSCC 89-98), blue (RHSCC 99-110) groups contained in Fan 2. Colors are selected from the range including 75C, 75D, 76A, 76B, 76C, 77C, 77D, 78D, N80D, N81B, 84C and colors in between or proximal thereto. In particular, genetically modified chrysanthemums are provided which now produce delphinidin. This is a novel feature of the genetically modified chrysanthemums and is encompassed by the term “elevated delphinidin” levels. The “elevation” is relative to zero or substantially zero levels in non-genetically modified chrysanthemum plants.

In one embodiment, the modified chrysanthemums comprise genetic sequences encoding a F3′5′H enzyme in combination with selected promoter and terminator sequences. In another embodiment, the chrysanthemum comprise genetic sequences encoding at least two F3′5′H. In yet another embodiment, the genetic sequences further encode a DFR and/or inhibit an indigenous F3′H. In yet another embodiment, the genetic sequences further encode a DFR and/or inhibit an indigenous F3′H and inhibit an indigenous DFR.

The chrysanthemums may be of any genetic background or hybrid background and include the Chrysanthemum sp. and Dendranthema sp species such as florist's chrysanthemum, Chrysanthemum×morylorium, Dendranthema grandiflora, Chrysanthemum aphrodite, Chrysanthemum arcticum, Chrysanthemum argyrophyllum, Chrysanthemum arisanense, Chrysanthemum boreale, Chrysanthemum chalchingolicum, Chrysanthemum chanetii, Chrysanthemum cinerariaefolium, Chrysanthemum coronarium, Crown daisy, Chrysanthemum crassum, Chrysanthemum glabriusculum, Chrysanthemum hypargyrum, Chrysanthemum indicum, Chrysanthemum japonense, Chrysanthemum japonicum, Chrysanthemum lavandulifolium, Chrysanthemum mawii, Chrysanthemum maximowiczii, Chrysanthemum mongolicum, Chrysanthemum morifoliwn, Chrysanthemum morii, Chrysanthemum okiense, Chrysanthemum oreastrum, Chrysanthemum ornatum, Chrysanthemum pacflcum, Chrysanthemum potentilloides, Chrysanthemum segetum, Chrysanthemum shiwogiku, Chrysanthemum sinuatum, Chrysanthemum vestitum, Chrysanthemum weyrichii, Chrysanthemum yoshinaganthum and Chrysanthemum zawadskii.

Particular varieties of interest include Blue Ridge, Madeline, Moneymaker, Mundial, Reagan and sports such as Improved Reagan and Dark Splendid Reagan, Sei Aida, Sei Figaro, Sei Faust, Sei Florea, Sei Spire, Sei Titan, Sei Titan 406, Sei Nouna, Sei Amelie and Mari Fusha.

Hence, one aspect of the present invention is directed to a genetically modified ornamental flowering plant exhibiting altered inflorescence in selected tissue, the ornamental plant comprising expressed genetic material encoding an F3′5′H enzyme in combination with a selected promoter and terminator.

The term “altered inflorescence” in this context means an inflorescence altered compared to the inflorescence of a plant (e.g. parent plant or a plant of the same species but not genetically modified) prior to genetic manipulation. The term “encoding” includes the expression of genetic material to produce a functional F3′5′H enzyme.

Another aspect of the present invention is directed to a chrysanthemum plant exhibiting altered inflorescence in selected tissue, the chrysanthemum comprising expressed genetic material encoding an F3′5′H enzyme in combination with a selected promoter and terminator.

Yet another aspect of the present invention is directed to a genetically modified chrysanthemum line or sport thereof exhibiting tissues of a red/purple to blue color including a violet/purple color, the chrysanthemum comprising an expressed genetic sequence encoding an F3′5′H enzyme in combination with a selected promoter and terminator.

Yet another aspect of the present invention provides a genetically modified chrysanthemum line or sport thereof exhibiting tissues of red/purple to blue color including a violet/purple color, the chrysanthemum comprising an expressed genetic sequence encoding an F3′5′H in combination with a selected promoter and terminator and an expressed genetic sequence encoding at least one other entity selected from the list comprising another F3′5′H, a DFR and an inhibitory nucleic acid sequence for an indigenous F3′H and/or DFR genetic sequence. An “indigenous” F3′H is an F3′H normally resident and expressed in the chrysanthemum parent line. An “indigenous” DFR is a DFR normally resident and expressed in the chrysanthemum parent line. This definition is the same for non-chrysanthemum plant species contemplated herein An “inhibitory” nucleic acid sequence includes inter alia antisense, sense, hairpin, double stranded RNA, RNAi and ribozyme.

Reference to a chrysanthemum comprising a genetic sequence means that the cells of the chrysanthemum plant contain the genetic material.

The present invention provides, therefore, a genetically modified chrysanthemum plant which comprises genetic material selected from the list comprising:

(i) a nucleotide sequence encoding an F3′5′H operably linked to a selected promoter and terminator;
(ii) a nucleotide sequence encoding an F3′5′H operably linked to a selected promoter and terminator and a heterologous DFR;
(iii) a nucleotide sequence encoding an F3′5′H operably linked to a selected promoter and terminator and a heterologous DFR and genetic material to down-regulate an indigenous F3′H;
(iv) a nucleotide sequence encoding an F3′5′H operably linked to a selected promoter and terminator and genetic material to down-regulate an indigenous DFR; and
(v) a nucleotide sequence encoding an F3′5′H operably linked to a selected promoter and terminator and a heterologous DFR and genetic material to down-regulate an indigenous F3′H and genetic material to down-regulate an indigenous DFR.

Progeny, reproductive material, cut flowers, tissue culturable cells and regenerable cells from the genetically modified ornamental flowering plants such as chrysanthemums also form part of the present invention.

The present invention is also directed to the use of a genetic sequence encoding an F3′5′H enzyme in the manufacture of a chrysanthemum or sports thereof exhibiting altered inflorescence including tissue having a red-purple to blue color including a violet/purple to blue color. The genetic sequence may also encode a second F3′5′H, a DFR or a nucleic acid molecule which inhibits expression of a F3′H genetic sequence, or a nucleic acid molecule which inhibits expression of a DFR genetic sequence.

The F3′5′H may be from any source. An F3′5′H enzyme from Viola sp, Cineraria sp, Petunia hybrida and Salvia sp is particularly useful. Suitable nucleotide sequences encoding an F3′5′H enzyme from Viola sp, Petunia hybrida, Cineraria sp and Salvia sp are set forth in Table 1.

The genetically modified chrysanthemum plants of the present invention produce enables production of delphinidin, a feature not present-based flavonoid molecules including delphinidin in the plants prior to genetic modificationchrysanthemums which are genetically modified to express a F3′5′H. This enables production of novel colors and altered inflorescence.

### BRIEF DESCRIPTION OF THE FIGURES

FIG. 1 is a schematic representation of the biosynthesis of anthocyanidin 3-glucosides that occur in most plants that produce anthocyanins. Enzymes involved in the pathway have been indicated as follows: PAL=Phenylalanine ammonia-lyase; C4H=Cinnamate 4-hydroxylase; 4CL=4-coumarate: CoA ligase; CHS=Chalcone synthase; CHI=Chalcone flavanone isomerase; F3H=Flavanone 3-hydroxylase; DFR=Dihydroflavonol-4-reductase; ANS=Anthocyanidin synthase, 3GT=UDP-glucose: flavonoid 3-O-glucosyltransferase. Other abbreviations include: DHK=dihydrokaempferol, DHQ=dihydroquercetin, DHM=dihydromyricetin.

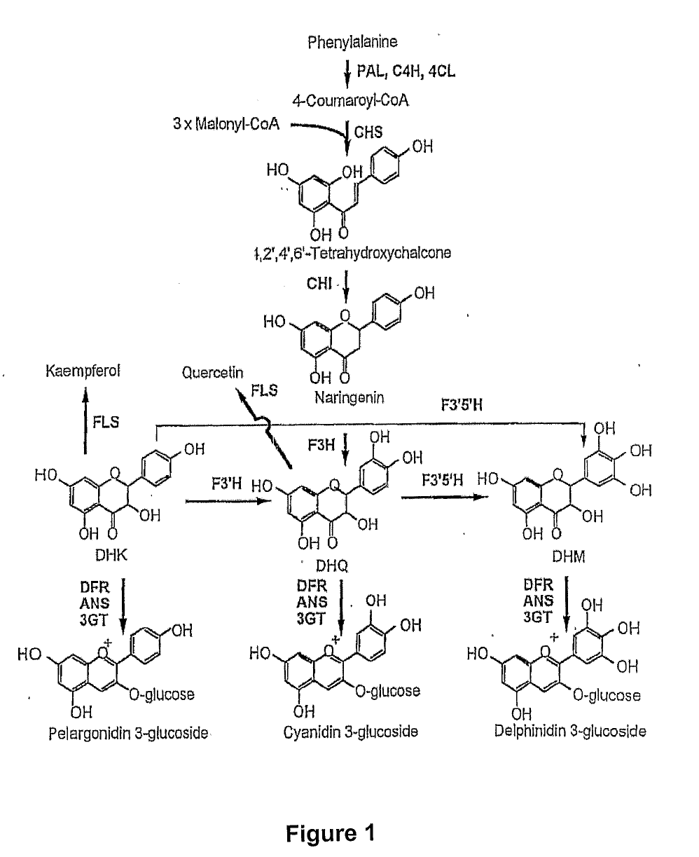

FIG. 2 is a diagrammatic representation of plasmid pCGP2205 comprising carnANS 5′: BPF3′5′H#18: carnANS 3′ and 35S 5′: SuRB expression cassettes. Abbreviations include: RB=right border of the T-DNA, LB=left border of the T-DNA, TetR=tetracycline resistance gene which confers resistance to the antibiotic tetracycline. See Table 3 for gene expression cassette details. Selected restriction endonuclease sites are marked.

FIG. 3 is a diagrammatic representation of plasmid pCGP2217 comprising RoseCHS 5′: BPF3′5′H#18: nos 3′ and 35S 5′: SuRB expression cassettes. Abbreviations include: RB=right border of the T-DNA, LB=left border of the T-DNA, TetR=tetracycline resistance gene which confers resistance to the antibiotic tetracycline. See Table 3 for gene expression cassette details. Selected restriction endonuclease sites are marked.

FIG. 4 is a diagrammatic representation of plasmid pCGP3424 comprising RoseCHS 5′: BPF3′5′H#18: nos 3, 35S 5′: ds clnysF3′H: ocs 3′ and 35S 5′: SuRB expression cassettes. Abbreviations include: RB=right border of the T-DNA, LB=left border of the T-DNA, TetR=tetracycline resistance gene which confers resistance to the antibiotic tetracycline. See Table 3 for gene expression cassette details.

FIG. 5 is a diagrammatic representation of plasmid pCGP3141 comprising Cinereria gF3′S′H and 35S 5′: SuRB expression cassettes. Abbreviations include: RB=right border of the T-DNA, LB=left border of the T-DNA, TetR=tetracycline resistance gene which confers resistance to the antibiotic tetracycline. See Table 3 for gene expression cassette details.

FIG. 6 is a diagrammatic representation of plasmid pCGP3429 comprising RoseCHS 5′: BPF3′5′H#18: nos 3, RoseCHS 5′: ds chrysF3′H*: nos 3′ and 35S 5′: SuRB expression cassettes. Abbreviations include: RB=right border of the T-DNA, LB=left border of the T-DNA, TetR=tetracycline resistance gene which confers resistance to the antibiotic tetracycline. See Table 3 for gene expression cassette details.

### DETAILED DESCRIPTION

As used in the subject specification, the singular forms “a”, “an” and “the” include plural aspects unless the context clearly dictates otherwise. Thus, for example, reference to “a plant” includes a single plant, as well as two or more plants; reference to “an F3′5′H enzyme” includes a single enzyme as well as two or more enzymes; reference to “the invention” includes a single aspect or multiple aspects of an invention; and so on.

The present invention contemplates genetically modified ornamental flowering plants and in particular chrysanthemums exhibiting altered including selected or desired inflorescence. The altered inflorescence may be in any tissue or organelle including flowers, petals, anthers and styles. Particular inflorescence contemplated herein includes a violet/purple to blue color. The color determination is conveniently measured against the Royal Horticultural Society (RHS) color chart and includes a color selected from or within the range of red-purple (RHSCC 58-74), purple (RHSCC 75-79), purple-violet (RHSCC 81-82), violet (RHSCC 83-88), violet-blue (RHSCC 89-98), blue (RHSCC 99-110) groups contained in Fan 2 of the RHS color chart. Particular colors include 75C, 75D, 76A, 76B, 76C, 77C, 78D, N80D, N81B, 84C and colors in between or proximal to either end of the above range. The term “inflorescence” is not to be narrowly construed and relates to any colored cells, tissues organelles or parts thereof.

Hence, one aspect of the present invention provides a genetically modified ornamental flowering plant exhibiting altered inflorescence compared to a plant of the same species prior to genetic modification, the genetically modified plant expressing a nucleotide sequence encoding an F3′5′H operably linked to a selected promoter and terminator.

Another aspect of the present invention provides a genetically modified ornamental flowering plant exhibiting altered inflorescence compared to a plant of the same species prior to genetic modification, the genetically modified plant expressing a nucleotide sequence encoding an F3′5′H operably linked to a selected promoter and terminator and further expressing a heterologous DFR nucleotide sequence.

A further aspect of the present invention provides a genetically modified ornamental flowering plant exhibiting altered inflorescence compared to a plant of the same species prior to genetic modification, the genetically modified plant expressing a nucleotide sequence encoding an F3′5′H operably linked to a selected promoter and terminator and further expressing a heterologous DFR nucleotide sequence and genetic material which down-regulates an indigenous F3′H.

Still another aspect of the present invention provides a genetically modified ornamental flowering plant exhibiting altered inflorescence compared to a plant of the same species prior to genetic modification, the genetically modified plant expressing a nucleotide sequence encoding an F3′5′H operably linked to a selected promoter and terminator and further expressing a heterologous DFR nucleotide sequence and genetic material which down-regulates an indigenous DFR.

Yet a further aspect of the present invention provides a genetically modified ornamental flowering plant exhibiting altered inflorescence compared to a plant of the same species prior to genetic modification, the genetically modified plant expressing a nucleotide sequence encoding an F3′5′H operably linked to a selected promoter and terminator and further expressing a heterologous DFR nucleotide sequence and genetic material which down-regulates an indigenous F3′H and an indigenous DFR.

In a particular embodiment, the ornamental flowering plant is a chrysanthemum.

Hence, another aspect of the present invention provides a genetically modified chrysanthemum exhibiting altered inflorescence compared to a plant of the same species prior to genetic modification, the genetically modified plant expressing a nucleotide sequence encoding an F3′5′H operably linked to a selected promoter and terminator.

Yet another aspect of the present invention provides a genetically modified chrysanthemum exhibiting altered inflorescence compared to a plant of the same species prior to genetic modification, the genetically modified plant expressing a nucleotide sequence encoding an F3′5′H operably linked to a selected promoter and terminator and further expressing a heterologous DFR nucleotide sequence.

A further aspect of the present invention provides a genetically modified chrysanthemum exhibiting altered inflorescence compared to a plant of the same species prior to genetic modification, the genetically modified plant expressing a nucleotide sequence encoding an F3′5′H operably linked to a selected promoter and terminator and further expressing a heterologous DFR nucleotide sequence and genetic material which down-regulates an indigenous F3′H.

Still another aspect of the present invention provides a genetically modified chrysanthemum exhibiting altered inflorescence compared to a plant of the same species prior to genetic modification, the genetically modified plant expressing a nucleotide sequence encoding an F3′5′H operably linked to a selected promoter and terminator and further expressing a heterologous DFR nucleotide sequence and genetic material which down-regulates an indigenous DFR.

Yet a further aspect of the present invention provides a genetically modified chrysanthemum exhibiting altered inflorescence compared to a plant of the same species prior to genetic modification, the genetically modified plant expressing a nucleotide sequence encoding an F3′5′H operably linked to a selected promoter and terminator and further expressing a heterologous DFR nucleotide sequence and genetic material which down-regulates an indigenous F3′H and an indigenous DFR.

The genetically modified chrysanthemums now produce delphinidin-based flavonoid molecules such as delphinidin. This enables alteration of inflorescence such as generation of purple/blue colors.

The present invention provides, therefore, a genetically modified chrysanthemum plant which comprises genetic material selected from the list comprising:

(i) a nucleotide sequence encoding an F3′5′H operably linked to a selected promoter and terminator;
(ii) a nucleotide sequence encoding an F3′5′H operably linked to a selected promoter and terminator and a heterologous DFR;
(iii) a nucleotide sequence encoding an F3′5′H operably linked to a selected promoter and terminator and a heterologous DFR and genetic material to down-regulate an indigenous F3′H;
(iv) a nucleotide sequence encoding an F3′5′H operably linked to a selected promoter and terminator and genetic material to down-regulate an indigenous DFR; and
(v) a nucleotide sequence encoding an F3′5′H operably linked to a selected promoter and terminator and a heterologous DFR and genetic material to down-regulate an indigenous F3′H and genetic material to down-regulate an indigenous DFR.

The genetically modified chrysanthemum plants of the present invention produce delphinidin which is not produced in non-genetically modified chrysanthemum plants. This is encompassed by the term “elevated delphinidin” levels.

Useful promoters include the promoters from flavonoid pathway genes such as CHS, ANS, F3′H, DFR, F3H, F3′5′H, F3′H, 3RT, MT, 3GT, AT genes, promoter sequence from the Rosa hybrida chalcone synthase (CHS) gene (Rose CHS 5′), promoter sequence from the Antirrhinum majus chalcone synthase (CHS) gene (AmCHS 5′), cauliflower mosaic virus 35S promoter (CaMV35S) or fragment thereof (e.g. 35S 5), promoter sequence from the chrysanthemum CHS (chrysCHS 5), a hybrid promoter consisting of the promoter from the mannopine synthase (mas) gene and CaMV35S enhancer region (Mac) Petunia hybrida phospholipid transfer protein gene promoter (petD8 5′), promoter sequence from the petunia anthocyanidin-3-glucoside rhamnosyltransferase (3RT) gene (petRT 5), a promoter sequence from the carnation ANS gene (carnANS 5), a promoter sequence from the cineraria F3′5′H gene (Cineraria F3′5′H 5), a promoter sequence from the petunia F3′5′H gene (Pet F3′5′H 5) a promoter sequence from the rose F3H gene (R. rugosa F3H 5), promoter sequence from the rose DFR gene (R. rugosa DFR 5), promoter sequence from the Viola F3′5′H gene (BPF3′5′H#40 5), or promoter sequence from the petunia DFR-A gene. Useful terminators include the terminator from mas gene of A. tumefaciens (mas 3′), terminator region from nos gene of A. tumefaciens (nos 3′), terminator fragment from octopine synthase gene (ocs 3′), phospholipid transfer protein gene terminator (petD8 3′), terminator sequence from the petunia 3RT gene (petRT 3), tumor morphology long gene terminator from A. tumefaciens (tml 3), a terminator sequence from carnation ANS gene (carnANS 3′), a terminator sequence from cineraria F3′S′H gene (Cineraria F3′5′H 3′), a terminator sequence from petunia F3′5′H gene (Pet F3′5′H 3′) or a terminator sequence from petunia DFR gene (petDFR 3′).

Accordingly, another aspect of the present invention is directed to a genetically modified chrysanthemum line or sports thereof exhibiting tissues of a red/purple to blue color including a violet/purple color, the chrysanthemum comprising genetic sequences encoding an F3′5′H enzyme operably linked to a promoter selected from the list comprising Antirrhinum majus chalcone synthase (CHS) gene promoter (AmCHS cauliflower mosaic virus 35S promoter (CaMV355) or fragment thereof (e.g. 35S 5′), CHS gene promoter from chrysanthemum (chrysCHS 5′), a hybrid promoter consisting of the promoter from the mannopine synthase (mas) gene and CaMV35S enhancer region (Mac), Petunia hybrida phospholipid transfer protein gene promoter (petD8 5′), anthocyanidin-3-glucoside rhamnosyltransferase (3RT) gene promoter (petRT5′), CHS gene promoter from rose (RoseCHS 5), and carnation ANS gene promoter (carnANS 5), cineraria F3′5′H gene promoter, petunia DFR gene promoter and operably linked to a terminator selected from the list comprising the mas gene of A. tumefaciens (mas 3), terminator region from nos gene of A. tumefaciens (nos 3), terminator fragment from octopine synthase gene (ocs 3′), phospholipid transfer protein gene terminator (petD8 3), 3RT gene terminator from Petunia (petRT 3′), tumor morphology long gene terminator from A. tumefaciens (tml 3′) or ANS gene terminator from carnation (carnANS 3), cineraria F3′5′H gene terminator, and a petunia DFR gene terminator.

Yet another aspect of the present invention is directed to a genetically modified chrysanthemum line or sports thereof exhibiting tissues of a red/purple to blue color including a violet/purple color, the chrysanthemum comprising genetic sequences encoding an F3′5′H enzyme operably linked to a promoter selected from the list comprising CHS, ANS, F3′H, DFR, F3H, F3′5′H, F3′H, 3RT, MT, 3GT, AT genes, promoter sequence from the Rosa hybrida chalcone synthase (CHS) gene (Rose CHS 5′), promoter sequence from the Antirrhinum majus chalcone synthase (CHS) gene (AmCHS 5′), cauliflower mosaic virus 35S promoter (CaMV35S) or fragment thereof (e.g. 35S 5′), promoter sequence from the chrysanthemum CHS (chrysCHS 5), a hybrid promoter consisting of the promoter from the mannopine synthase (mas) gene and CaMV35S enhancer region (Mac), Petunia hybrida phospholipid transfer protein gene promoter (petD8 5′), promoter sequence from the petunia anthocyanidin-3-glucoside rhamnosyltransferase (3RT) gene (petRT 5), a promoter sequence from the carnation ANS gene (carnANS 5′), a promoter sequence from the cineraria F3′5′H gene (Cineraria F3′5′H 5), a promoter sequence from the petunia F3′5′H gene (Pet F3′5′H 5) a promoter sequence from the rose F3H gene (R. rugosa F3H 5), promoter sequence from the rose DFR gene (R. rugosa DFR 5), promoter sequence from the Viola F3′5′H gene (BPF3′5′H#40 5), or promoter sequence from the petunia DFR-A gene and operably linked to a terminator sequence selected from the list comprising petunia 3RT gene (petRT 3), tumor morphology long gene terminator from A. tumefaciens (tml 3), a terminator sequence from carnation ANS gene (carnANS 3′), a terminator sequence from cineraria F3′5′H gene (Cineraria F3′5′H 3′), a terminator sequence from petunia F3′5′H gene (Pet F3′5′H 3′) and a terminator sequence from petunia DFR gene.

Non-flavonoid pathway gene promoters such as the 35S promoter may also be used as well as non-flavonoid pathway gene terminators. In addition, a non-flavonoid pathway gene promoter (e.g. 35S promoter) may be used with a flavonoid pathway gene terminator. Alternatively, a flavonoid pathway gene promoter (e.g. as exemplified above) may be used with a non-flavonoid pathway gene terminator. Reference to “35S promoter” includes 35S 5′ or CaMV 35S.

The genetic sequence is conveniently a single construct carrying the nucleotide sequences encoding the F3′5′H enzyme or two F3′5′H enzymes operably linked to the promoter and terminator. In addition, the genetic sequence may be integrated into the genome of a plant cell or it may be maintained as an extra-chromosomal artificial chromosome. Still furthermore, the generation of a chrysanthemum expressing an F3′5′H enzyme may be generated by recombinant means alone or by a combination of conventional breeding and recombinant DNA manipulation.

Hence, the present invention contemplates a method for producing a chrysanthemum exhibiting altered inflorescence, the method comprising introducing into regenerable cells of a chrysanthemum plant, genetic material encoding an F3′5′H enzyme operably linked to a selected promoter and a terminator and regenerating a plant therefrom or genetically modified progeny thereof.

Another aspect of the present invention contemplates a method for producing a chrysanthemum exhibiting altered inflorescence, the method comprising introducing into regenerable cells of a chrysanthemum plant, genetic material encoding an F3′5′H enzyme operably linked to a selected promoter and a terminator and genetic material which down-regulates an indigenous F3′H and regenerating a plant therefrom or genetically modified progeny thereof.

Yet another aspect of the present invention contemplates a method for producing a chrysanthemum exhibiting altered inflorescence, the method comprising introducing into regenerable cells of a chrysanthemum plant, genetic material encoding an F3′5′H enzyme operably linked to a selected promoter and a terminator and genetic material expressing a heterologous DFR and regenerating a plant therefrom or genetically modified progeny thereof.

Still another aspect of the present invention contemplates a method for producing a chrysanthemum exhibiting altered inflorescence, the method comprising introducing into regenerable cells of a chrysanthemum plant, genetic material encoding an F3′5′H enzyme operably linked to a selected promoter and a terminator and genetic material expressing a heterologous DFR and genetic material which down-regulates an indigenous DFR and regenerating a plant therefrom or genetically modified progeny thereof.

Even yet another aspect of the present invention contemplates a method for producing a chrysanthemum exhibiting altered inflorescence, the method comprising introducing into regenerable cells of a chrysanthemum plant, genetic material encoding an F3′5′H enzyme operably linked to a selected promoter and a terminator and genetic material expressing a heterologous DFR and genetic material which down-regulates an indigenous F3′H and an indigenous DFR.

The genetic mutant is operably linked to a promoter to facilitate expression. The genetically manipulated plant may also then undergo various generations of growth or cultivation. Hence, reference to a “genetically modified chrysanthemum” includes progeny thereof and sister lines thereof as well as sports thereof. The present invention also extends to reproductive material from the genetically modified chrysanthemum.

Another aspect of the present invention provides a method for producing a chrysanthemum line exhibiting altered inflorescence, the method comprising selecting a chrysanthemum comprising genetic material encoding an F3′5′H enzyme and operably linked to a selected promoter and terminator and crossing this plant with another chrysanthemum having a desired genetic background and then selecting F1 or subsequent generation plants. The “desired genetic background” includes flower color, rigor, vacuolar pH and the like.

The present invention further contemplates a method for producing a chrysanthemum line exhibiting altered inflorescence, the method comprising selecting a chrysanthemum comprising genetic material encoding an F3′5′H enzyme operably linked to a selected promoter and a terminator and genetic material which down-regulates an indigenous F3′H and regenerating a plant therefrom or genetically modified progeny thereof and crossing this plant with another chrysanthemum having a desired genetic background and then selecting F1 or subsequent generation plants.

Yet another aspect of the present invention contemplates a method for producing a chrysanthemum exhibiting altered inflorescence, the method comprising introducing into regenerable cells of a chrysanthemum plant, genetic material encoding an F3′5′H enzyme operably linked to a selected promoter and a terminator and genetic material expressing a heterologous DFR and regenerating a plant therefrom or genetically modified progeny thereof.

Still another aspect of the present invention contemplates a method for producing a chrysanthemum exhibiting altered inflorescence, the method comprising introducing into regenerable cells of a chrysanthemum plant, genetic material encoding an F3′5′H enzyme operably linked to a selected promoter and a terminator and genetic material expressing a heterologous DFR and genetic material which down-regulates an indigenous DFR and regenerating a plant therefrom or genetically modified progeny thereof.

Even yet another aspect of the present invention contemplates a method for producing a chrysanthemum exhibiting altered inflorescence, the method comprising introducing into regenerable cells of a chrysanthemum plant, genetic material encoding an F3′5′H enzyme operably linked to a selected promoter and a terminator and genetic material expressing a heterologous DFR and genetic material which down-regulates an indigenous F3′H and an indigenous DFR.

The F3′5′H enzyme may be from any source including Viola sp, Petunia sp, Cineraria sp, Salvia sp, Viola sp, Lisianthus sp, Gentiana sp, Sollya sp, Salvia sp, Clitoria sp, Kennedia sp, Campanula sp, Lavandula sp, Verbena sp, Torenia sp, Delphinium sp, Solanum sp, Cineraria sp, Vitis sp, Babiana stricta, Pinus sp, Picea sp, Larix sp, Phaseolus sp, Vaccinium sp, Cyclamen sp, Iris sp, Pelargonium sp, Liparieae, Geranium sp, Pisum sp, Lathyrus sp, Catharanthus sp, Malvia sp, Mucuna sp, Vicia sp, Saintpaulia sp, Lagerstroemia sp, bouchina sp, Plumbago sp, Hypocalyptus sp, Rhododendron sp, Linum sp, Macroptilium sp, Hibiscus sp, Hydrangea sp, Cymbidium sp, Millettia sp, Hedysarum sp, Lespedeza sp, Asparagus sp, Antigonon sp, Pisum sp, Freesia sp, Brunella sp, Clarkia sp, etc. Particularly useful F3′5′H enzymes are from black pansy (Viola sp), Cineraria sp, Petunia hybrida or Salvia sp.

The presence of F3′5′H enzymes leads to the pressure of production of delphinidin in the plant. In chrysanthemum plants, this feature is a novel outcome. Delphinidin and delphinidin-based flavonoid molecules is not otherwise observed normally produced in non-genetically modified chrysanthemum plants. This facilitates production of colors new to chrysanthemum plants.

The present invention further contemplates the use of genetic sequences encoding an F3′5′H enzyme and in the manufacture of a chrysanthemum or sports thereof exhibiting altered inflorescence including tissue having a red/purple to blue color including a violet/purple color.

The present invention further contemplates the use of genetic sequences encoding an F3′5′H enzyme and also which down-regulate expression of an indigenous F3′H in the manufacture of a chrysanthemum or sports thereof exhibiting altered inflorescence including tissue having a red/purple to blue color including a violet/purple color.

The present invention also provides the use of genetic sequences which encode an F3′5′H and a heterologous DFR and also which down-regulate expression of an indigenous F3′H and/or a DFR in the manufacture of a chrysanthemum or sports thereof exhibiting altered inflorescence including tissue having a red/purple to blue color including a violet/purple color.

Generally, the genetic sequences comprise a selected promoter and terminator.

Cut flowers, tissue culturable cells, regenerable cells, parts of plants, seeds, reproductive material (including pollen) are all encompassed by the present invention.

Nucleic acid molecules encoding F3′5′Hs are also provided in International Patent Application No. PCT/AU92/00334 and Holton et al, 1993 supra. These sequences have been used to modulate 3′, 5′ hydroxylation of flavonoids in petunia (see International Patent Application No. PCT/AU92/00334 and Holton et al, 1993, supra), tobacco (see International Patent Application No. PCT/AU92/00334) and carnations (see International Patent Application No. PCT/AU96/00296). Nucleotide sequences of F3′5′H from other species such as Viola, Salvia and Sonya have been cloned (see International Patent Application No. PCT/AU03/01111). Any of these sequences may be used in combination with a promoter and/or terminator. The present invention particularly contemplates a F3′5′H encoded by SEQ ID NOs:1 or 3 or 5 or 7 or 12 (see Table 1) or a nucleotide sequence capable of hybridizing to any of SEQ ID NOs:1, 3, 5, 7 or 12 or a complementary form thereof under low or high stringency conditions or which has at least about 70% identity to SEQ ID NO:1 or 3 or 5 or 7 or 12 after optimal alignment. In one particular embodiment, the nucleic acid molecule encoding an F3′5′H is in construct pCGP2217 which comprises RoseCHS 5′: BPF3′5′H#18: nos 3′. In another embodiment, the nucleic acid molecule encoding an F3′5′H is in construct pCGP3424 which comprises RoseCHS 5′: BPF3′5′H#18: nos 3′. 35S 5′: dschrysF3′H: ocs 3′. In another embodiment the nucleic acid molecule encoding an F3′5′H is in construct pCGP3429 which comprises RoseCHS 5′: BPF3′5′H#18: nos 3′. RoseCHS 5′: dschrysF3′H*: nos 3′. In another embodiment, the nucleic acid molecule encoding an F3′5′H is in construct pCGP2205 which comprises carnANS 5: BPF3′5′H#18: carnANS 3′. In yet another embodiment, the nucleic acid molecule encoding an F3′5′H is in construct pCGP3141 which comprises Cineraria gF3′5′H. Such plants produce delphinidin as detected by TLC or HPLC. This may be referred to as “elevated” levels of delphinidin. It is elevated relative to substantially zero amounts of delphinidin in non-genetically modified plants.

For the purposes of determining the level of stringency to define nucleic acid molecules capable of hybridizing to SEQ ID NO:1, SEQ ID NO:3 or SEQ ID NO:5 or SEQ ID NO:7 or SEQ ID NO:12 or their complementary forms, low stringency includes and encompasses from at least about 0% to at least about 15% v/v formamide and from at least about 1M to at least about 2 M salt for hybridization, and at least about 1 M to at least about 2 M salt for washing conditions. Generally, low stringency is from about 25-30° C. to about 42° C. The temperature may be altered and higher temperatures used to replace the inclusion of formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % (Marmur and Doty, J. Mol. Biol. 5:109, 1962). However, the Tₘ of a duplex DNA decreases by 1° C. with every increase of 1% in the number of mismatched base pairs (Bonner and Laskey, Eur. J. Biochem. 46:83, 1974). Formamide is optional in these hybridization conditions. Accordingly, particularly preferred levels of stringency are defined as follows: low stringency is 6×SSC buffer, 1.0% w/v SDS at 25-42° C.; a moderate stringency is 2×SSC buffer, 1.0% w/v SDS at a temperature in the range 20° C. to 65° C.; high stringency is 0.1×SSC buffer, 0.1% w/v SDS at a temperature of at least 65° C.

Reference to at least 70% identity includes 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100% identity. The comparison may also be made at the level of similarity of amino acid sequences of SEQ ID NOs:2, 4, 6, 8 or 13. Hence, nucleic acid molecules are contemplated herein which encode an F3′S′H enzyme having at least 70% similarity to the amino acid sequence set forth in SEQ ID NOs:2 or 4 or 6 or 8 or 13. Again, at least 70% similarity includes 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100% similarity or identity.

The nucleic acid molecule encoding the F3′S′H enzyme includes one or more promoters and/or terminators. In one embodiment, a promoter is selected which directs expression of a F3′S′H nucleotide sequence in tissue having a higher pH.

In an embodiment, the promoter sequence is native to the host chrysanthemum plant to be transformed or may be derived from an alternative source, where the region is functional in the host plant. Other sources include the Agrobacterium T-DNA genes, such as the promoters of genes encoding enzymes for biosynthesis of nopaline, octapine, mannopine, or other opines; promoters from plants, such as promoters from genes encoding ubiquitin; tissue specific promoters (see, e.g, U.S. Pat. No. 5,459,252 to Conkling et al; WO 91/13992 to Advanced Technologies); promoters from plant viruses (including host specific viruses), or partially or wholly synthetic promoters. Numerous promoters that are potentially functional in chrysanthemum plants (see, for example, Greve, J. Mol. Appl. Genet. 1:499-511, 1983; Salomon et al, EMBO, J. 3:141-146, 1984; Garfinkel et al, Cell 27:143-153, 1981; Barker et al, Plant Mol. Biol. 2:235-350, 1983); including various promoters isolated from plants (such as the Ubi promoter from the maize obi-1 gene, see, e.g, U.S. Pat. No. 4,962,028) and plant viruses (such as the cauliflower mosaic virus promoter, CaMV 35S). In other embodiments the promoter is 35S 5′, RoseCHS 5′, AmCHS 5′, chrysCHS 5′, petD8 5′, petRT 5′, carnANS 5′, cineraria F3′5′H 5, BPF3′5′H#40 5, R. rugosa DFR 5, R. rugosa F3H 5′ and/or promoter region from the DFR gene of petunia with corresponding terminators pet D8 3′, nos 3, ocs 3, carnANS 3′ and petDFR 3′ (terminator region from the DFR gene from petunia).

The promoter sequences may include cis-acting sequences which regulate transcription, where the regulation involves, for example, chemical or physical repression or induction (e.g, regulation based on metabolites, light, or other physicochemical factors; see, e.g, WO 93/06710 disclosing a nematode responsive promoter) or regulation based on cell differentiation (such as associated with leaves, roots, seed, or the like in plants; see, e.g. U.S. Pat. No. 5,459,252 disclosing a root-specific promoter).

Other cis-acting sequences which may be employed include transcriptional and/or translational enhancers. These enhancer regions are well known to persons skilled in the art, and can include the ATG initiation codon and adjacent sequences.

The nucleic acid molecule encoding an F3′5′H enzyme, in combination with suitable promoters and/or terminators is/are used to modulate the level of a flavonoid molecule such as delphinidin in a chrysanthemum. Modulating the level of a delphinidin-based molecule relates to an elevation or reduction in levels of up to 30% or more particularly of 30-50%, or even more particularly 50-75% or still more particularly 75% or greater above or below the normal endogenous or existing levels of activity. Conveniently delphinidin levels are detected by TLC or HPLC although any detection means is contemplated herein.

Hence, the present invention provides a genetically modified chrysanthemum plant comprising genetic material which is expressed to produce an F3′5′H enzyme, which produces delphinidin in tissue which contributes to the tissue being a color in the range red/purple to blue color including a violet/purple color.

The present invention further provides a genetically modified chrysanthemum plant comprising genetic material which is expressed to produce an F3′5′H enzyme and a heterologous DFR, which plant produces delphinidin in tissue which contributes to the tissue being a color in the range red/purple to blue color including a violet/purple color.

Another aspect of the present invention is directed to a genetically modified chrysanthemum plant comprising genetic material which is expressed to produce an F3′5′H enzyme and a down-regulated indigenous F3′H, which plant produces delphinidin in tissue which contributes to the tissue being a color in the range red/purple to blue color including a violet/purple color.

Yet another aspect of the present invention relates to a genetically modified chrysanthemum plant comprising genetic material which is expressed to produce an F3′5′H enzyme and a heterologous DFR and a down-regulated indigenous DFR, which plant produces delphinidin in tissue which contributes to the tissue being a color selected from a red/purple to blue color including a violet/purple color.

Still another aspect of the present invention provides a genetically modified chrysanthemum plant comprising genetic material which is expressed to produce an F3′5′H enzyme and a heterologous DFR and a down-regulated indigenous DFR and a down-regulated F3′H, which plant produces delphinidin in tissue which contributes to the tissue being a color in the range red/purple to blue color including a violet/purple color.

The term “inflorescence” as used herein refers to the flowering part of a plant or any flowering system of more than one flower which is usually separated from the vegetative parts by an extended internode, and normally comprises individual flowers, bracts and peduncles, and pedicels. As indicated above, reference to a “transgenic plant” may also be read as a “genetically modified plant” and includes a progeny or hybrid line ultimately derived from a first generation transgenic plant.

The present invention also contemplates the use of a genetic sequence encoding an F3′5′H enzyme operably linked to a selected promoter and terminator in the manufacture of a chrysanthemum plant or sports thereof exhibiting altered inflorescence including tissue having a red/purple to blue color including a violet/purple color and which plant comprises elevated levels of delphinidin.

Another aspect of the present invention contemplates the use of a genetic sequence encoding an F3′5′H enzyme operably linked to a selected promoter and a terminator and genetic material expressing a heterologous DFR in the manufacture of a chrysanthemum plant or sports thereof exhibiting altered inflorescence including tissue having a red/purple to blue color including a violet/purple color and which plant comprises elevated levels of delphinidin.

Yet another aspect of the present invention is directed to a use of genetic material encoding an F3′5′H enzyme operably linked to a selected promoter and a terminator and genetic material which down-regulates an indigenous F3′H in the manufacture of a chrysanthemum plant or sports thereof exhibiting altered inflorescence including tissue having a red/purple to blue color including a violet/purple color and which plant comprises elevated levels of delphinidin.

Still another aspect of the present invention contemplates use of genetic material encoding an F3′5′H enzyme operably linked to a selected promoter and a terminator and genetic material expressing a heterologous DFR and genetic material which down-regulates an indigenous DFR and in the manufacture of a chrysanthemum plant or sports thereof exhibiting altered inflorescence including tissue having a red/purple to blue color including a violet/purple color and which plant comprises elevated levels of delphinidin.

Even yet another aspect of the present invention provides a use of genetic material encoding an F3′5′H enzyme operably linked to a selected promoter and a terminator and genetic material expressing a heterologous DFR and genetic material which down-regulates an indigenous F3′H and an indigenous DFR the manufacture of a chrysanthemum plant or sports thereof exhibiting altered inflorescence including tissue having a red/purple to blue color including a violet/purple color and which plant comprises elevated levels of delphinidin.

The present invention represents a new approach to developing plant varieties and in particular chrysanthemums having altered color characteristics. In addition, the present invention enables the generation of extracts of F3′5′H transformed plants and in particular genetically modified chrysanthemum paints wherein the extract has use, for example, as a flavoring or food additive or health product or beverage or juice or coloring. Beverages may include but are not limited to teas, wines, spirits, coffee, milk and dairy products.

Reference to an “extract” includes a purified or substantially purified fraction or molecule or delphinidin-containing portion.

Another aspect of the present invention contemplates the use of the extracts from genetically modified plants or plant parts of genetically modified plants or progeny of the genetically modified paints containing all or part of the nucleic acid sequences of the present invention and, in particular, the extracts from those genetically modified plants when used as a flavoring or food additive or health product or beverage or juice or coloring. In particular, the genetically modified plant is a chrysanthemum producing delphinidin-based flavonoid compounds or molecules such as delphinidin.

In addition, the development of novel colored varieties of plant parts such as seeds offer significant opportunities in agriculture. For example, novel colored chrysanthemum seeds are useful as proprietary tags for plants.

The present invention is further described by the following non-limiting Example. In this Example, materials and methods as outlined below were employed:

Methods followed were as described in Sambrook et al, Molecular Cloning: A Laboratory Manual, Cold Spring Harbor Laboratories, Cold Spring Harbor, N.Y., USA, 1989 or Sambrook and Russell, Molecular Cloning: A Laboratory Manual 3^{rd} edition, Cold Spring Harbor Laboratories, Cold Spring Harbor, N.Y., USA, 2001 or Plant Molecular Biology Manual (2^{nd} edition), Gelvin and Schilperoot (eds), Kluwer Academic Publisher, The Netherlands, 1994 or Plant Molecular Biology Labfax, Croy (ed), Bios scientific Publishers, Oxford, UK, 1993.

The cloning vectors pBluescript and PCR script were obtained from Stratagene, USA. pCR72.1 was obtained from Invitrogen, USA.

E. coli Transformation

The Escherichia coli strains used were:

### DH5α

supE44, Δ (lacZYA-ArgF)U169, (ø80lacZAM15), hsdR17(rₖ⁻, mₖ⁺),
recA1, endA1, gyrA96, thi-1, relA1, deoR. (Hanahan, J. Mol. Biol. 166:557, 1983)

### XL1-Blue

supE44, hsdR17(rₖ⁻, mₖ⁺), recA1, endA1, gyrA96, thi-1, relA1,
lac⁻, [F′proAB, lacI^{q}, lacZΔM15, Tn10(tet^{R})] (Bullock et al, Biotechniques 5:376, 1987).

### BL21-CodonPlus-RIL Strain

ompT hsdS(Rb-mB-) dcm+ Tet^{r} gal endA Hte [argU ileY leuW Cam^{r}]
M15 E. coli is derived from E. coli K12 and has the phenotype Nal^{s}, Str^{s}, Thi⁻, Ara⁺, Gal⁺, Mtl⁻, F⁻, RecA⁺, Uvr⁺, Lon⁺. Transformation of the E. coli strains was performed according to the method of Inoue et al, (Gene 96:23-28, 1990).
Agrobacterium tumefaciens Strains and Transformations

The disarmed Agrobacterium tumefaciens strain used was AGLO (Lazo et al, Bio/technology 9:963-967, 1991).

Plasmid DNA was introduced into the Agrobacterium tumefaciens strain AGLO by adding 5 μg of plasmid DNA to 100 μL of competent AGLO cells prepared by inoculating a 50 mL LB culture (Sambrook et al, 1989, supra) and incubation for 16 hours with shaking at 28° C. The cells were then pelleted and resuspended in 0.5 mL of 85% (v/v) 100 mM CaCl₂/15% (v/v) glycerol. The DNA-Agrobacterium mixture was frozen by incubation in liquid N₂ for 2 minutes and then allowed to thaw by incubation at 37° C. for 5 minutes. The DNA/bacterial mix was then placed on ice for a further 10 minutes. The cells were then mixed with 1 mL of LB (Sambrook et al, 1989 supra) media and incubated with shaking for 16 hours at 28° C. Cells of A. tumefaciens carrying the plasmid were selected on LB agar plates containing appropriate antibiotics such as 50 μg/mL tetracycline or 100 μg/mL gentamycin. The confirmation of the plasmid in A. tumefaciens was by restriction endonuclease mapping of DNA isolated from the antibiotic-resistant transformants.

### DNA Ligations

DNA ligations were carried out using the Amersham Ligation Kit or Promega Ligation Kit according to procedures recommended by the manufacturer.

### Isolation and Purification of DNA Fragments

Fragments were generally isolated on a 1% (w/v) agarose gel and purified using the QIAEX II Gel Extraction kit (Qiagen) or Bresaclean Kit (Bresatec, Australia) following procedures recommended by the manufacturer.

Repair of Overhanging Ends after Restriction Endonuclease Digestion

Overhanging 5′ ends were repaired using DNA polymerase I Klenow fragment according to standard protocols (Sambrook et al, 1989 supra). Overhanging 3′ ends were repaired using Bacteriophage T4 DNA polymerase according to standard protocols (Sambrook et al, 1989 supra).

Removal of Phosphoryl Groups from Nucleic Acids

Shrimp alkaline phosphatase (SAP) [USB] was typically used to remove phosphoryl groups from cloning vectors to prevent re-circularization according to the manufacturer's recommendations.

### Polymerase Chain Reaction (PCR)

Unless otherwise specified, PCR conditions using plasmid DNA as template included using 2 ng of plasmid DNA, 100 ng of each primer, 2 μL 10 mM dNTP mix, 5 μL 10×Taq DNA polymerase buffer, 0.5 μL Taq DNA Polymerase in a total volume of 50 μL. Cycling conditions comprised an initial denaturation step of 5 minutes at 94° C., followed by 35 cycles of 94° C. for 20 sec, 50° C. for 30 sec and 72° C. for 1 minute with a final treatment at 72° C. for 10 minutes before storage at 4° C.

PCRs were performed in a Perkin Elmer GeneAmp PCR System 9600.

### ³²P-Labeling of DNA Probes

DNA fragments were radioactively labeled with 50 μCi of [α-³²P]-dCTP using a Gigaprime kit (Geneworks, AUSTRALIA) or a Decaprime kit (AMBION, USA), following methods recommended by the manufacturer. Unincorporated [α-³²P]-dCTP was removed by chromatography on Sephadex G-50 (Fine) columns or Microbiospin P-30 Tris chromatography columns (BioRad).

### Plasmid Isolation

Single colonies were analyzed for inserts by inoculating LB broth (Sambrook et al, 1989, supra) with appropriate antibiotic selection (e.g. 100 μg/mL ampicillin or 10 to 50 μg/mL tetracycline etc.) and incubating the liquid culture at 37° C. (for E. coli) or 29° C. (for A. tumefaciens) for ˜16 hours with shaking. Plasmid DNA was purified using the alkali-lysis procedure (Sambrook et al, 1989, supra) or using The WizardPlus SV minipreps DNA purification system (Promega) or Qiagen Plasmid Mini Kit (Qiagen). Once the presence of an insert had been determined, larger amounts of plasmid DNA were prepared from 50 mL overnight cultures using the alkali-lysis procedure (Sambrook et al, 1989, supra) or QIAfilter Plasmid Midi kit (Qiagen) and following conditions recommended by the manufacturer.

### DNA Sequence Analysis

DNA sequencing was performed using the PRISM (trademark) Ready Reaction Dye Primer Cycle Sequencing Kits from Applied Biosystems. The protocols supplied by the manufacturer were followed. The cycle sequencing reactions were performed using a Perkin Elmer PCR machine (GeneAmp PCR System 9600). Sequencing runs were generally performed by the Australian Genome Research Facility at the University of Queensland, St Lucia, Brisbane, Australia and at The Walter and Eliza Hall Institute of Medical Research (Melbourne, Australia).

Sequences were analysed using a MacVector (Trade mark) application (version 10.0.2 and earlier) [MacVector Inc, Cary, N.C., USA].

Homology searches against Genbank, SWISS-PROT and EMBL databases were performed using the FASTA and TFASTA programs (Pearson and Lipman, Proc. Natl. Acad. Sci. USA 85(8):2444-2448, 1988) or BLAST programs (Altschul et al, J. Mol. Biol. 215(3):403-410, 1990). Percentage sequence similarities were obtained using LALIGN program (Huang and Miller, Adv. Appl. Math. 12:373-381, 1991) or ClustalW program (Thompson et al, Nucleic Acids Research 22:4673-4680, 1994) within the MacVector (Trade mark) application (MacVector Inc, USA) using default settings.

Multiple sequence alignments were produced using ClustalW (Thompson et al, 1994, supra) using default settings.

### Plant Transformations

Plant transformations are as described in International Patent Application No. PCT/US91/05805, U.S. Pat. No. 251,392 (US Registration No. 5,567,599) using appropriate selection regimes depending on the selectable marker gene cassette. Kanamycin selection for nptII selectable marker genes and chlorsulfuron selection for SuRB selectable marker genes Other methods may also be employed. For example, Aida et al, Breeding Science, 54:51-58, 2004; Dolgov et al, Acta Hort 447, 329-334, 1997; Toguri et al, Plant Biotechnology, 20:121-127, 2003; Ledger et al, Plant Cell Reports, 10:195-199, 1991; da Silva, Biotechnology Advances 21, 715-766, 2003.

Chrysanthemum varieties were obtained from CleanGrow Australia or Seikoen, Japan.

### Transgenic Analysis

### Color Coding

The Royal Horticultural Society's Color Charts, Third and/or Fifth edition (London, UK), 1995 and/or 2007 were used to provide a description of observed color. They provide an alternative means by which to describe the color phenotypes observed. The designated numbers, however, should be taken only as a guide to the perceived colors and should not be regarded as limiting the possible colors which may be obtained.

Chrysanthemum inflorescences generally consist of many flowers called florets. There are generally two kinds of florets, ray florets and disk florets. On a daisy type of chrysanthemum such as Improved Reagan, the outer parts are ray or ligulate florets and the petals contribute to the majority of the color observed and the center is composed of disc (disk) or tubular florets (National Chrysanthemum Society, Inc., USA, 2001, Glimn-Lacy and Kaufman, Botany Illustrated, Introduction to Plants, Major Groups, Flowering Plant Families, 2^{nd} ed, Springer, USA, 2006)

The petal colors reported in this specification are from the ray or ligulate florets.

### Chromatographic Analysis

Thin Layer Chromatography (TLC) and High Performance Liquid Chromatography (HPLC) analysis was performed generally as described in Brugliera et al, (Plant J. 5:81-92, 1994). These techniques enable detection of delphinidin-based flavonoid molecules such as delphinidin.

### Extraction of Anthocyanidins

Prior to HPLC analysis, the anthocyanin and flavonol molecules present in petal and stamen extracts were acid hydrolysed to remove glycosyl moieties from the anthocyanidin or flavonol core. Anthocyanidin and flavonol standards were used to help identify the compounds present in the floral extracts.

Anthocyanidins in the reaction mixture were analysed by HPLC via gradient elution using gradient conditions of 50% B to 60% B over 10 minutes, then 60% B for 10 minutes and finally 60% B to 100% B over 5 minutes where solvent A consisted of TFA: H₂O (5:995) and solvent B consisted of acetonitrile: TFA: H₂O (500:5:495). An Asahi Pac ODP-50 cartridge column (250 mm×4.6 mm ID) was used for the reversed phase chromatographic separations. The flow rate was 1 mL/min and the temperature was 40° C. The detection of the anthocyanidin compounds was carried out using a Shimadzu SPD-M6A three dimensional detector at 400-650 nm.

The anthocyanidin peaks were identified by reference to known standards, viz delphinidin, petunidin, malvidin, cyanidin and peonidin

### Stages of Flower Development

Chrysanthemum inflorescences were harvested at developmental stages defined as follows:

Stage 1: Closed bud, petals not visible.
Stage 2: Inflorescence buds opening: tips of petals visible.
Stage 3: Tips of nearly all petals exposed.
Stage 4: Outer petals at 45° angle to stem.
Stage 5: Inflorescence fully open.

For TLC or HPLC analysis, ray petals were collected from stage 4 inflorescences at the stage of maximum pigment accumulation.

For Northern blot analysis, ray petals were collected from stage 3 to 4 inflorescences at the stage of maximal expression of flavonoid pathway genes.

The Petunia hybrida cultivars used are presented in Table 2.

TABLE 2
Genotypes of Petunia hybrida cultivars

| Plant | | |
|---|---|---|
| variety | Properties | Source/Reference |
| V23 | An1, An2, An3, An4, An6, | Wallroth et al. (Mol. Gen. |
| | An8, An9, An10, ph1, Hf1, | Genet. 202: 6-15, 1986) |
| | Hf2, ht1, Rt, po, Bl, Fl | Doodeman et al. (Theor. |
| | | Appl. Genet. (57: 357-366, |
| | | 1984) |
| Old Glory | F₁ Hybrid | Ball Seed, USA |
| Blue (OGB) | (commercial cultivar) | |
| Th7 | An1, An2, An3, An4, An6, | INRA |
| | An9, An10, An11, Hf1, Hf2, | |
| | Ht1, Ht2, Ph1, Ph2, Ph5, | |
| | Rt, po, mf1, mf2, Gf, fl | |

INRA = Institut National de la Recherche Agronomique, Cedex, France

### Northern/RNA Blot Analysis

Transcription of a transferred gene was monitored by isolating RNA and estimating the quantity and size of the expected transcript. Northern blot analysis was used to monitor the steady-state level of particular transcripts in petals. A transcript was determined to be intact or full-length based on the estimated size expected from the gene used. In general when cDNAs were used as coding sequences the size of the transcript expected would be the size of the cDNA plus any 5′ untranslated component of the fused promoter fragment plus any 3′ untranslated sequence from the fused terminator fragment. In some cases where a cDNA region contained a putative polyadenylation site and the terminator region contained a putative polyadenylation site, 2 transcripts would be detected. One would be of a size consistent with polyadenylation occurring just downstream from the polyadenylation site within the cDNA sequence. The second transcript would be larger and consistent with the transcript being polyadenylated after the polyadenylation site within the terminator fragment.

Total RNA was isolated from petals or leaves using a Plant RNAeasy kit (QIAGEN) following procedures recommended by the Manufacturer or using the method of Turpen and Griffith (BioTechniques 4:11-15, 1986).

RNA samples (5 μg) were electrophoresed through 2.2 M formaldehyde/1.2% w/v agarose gels using miming buffer containing 40 mM morpholinopropanesulphonic acid (pH 7.0), 5 mM sodium acetate, 0.1 mM EDTA (pH 8.0). The RNA was stained with ethidium bromide and visualized under UV-light. The ribosomal RNA was generally used as a guide in confirming that the RNA had not been degraded by intra- or extra-cellular ribonucleases. The RNA was transferred to Hybond-N membrane filters (Amersham) and treated as described by the manufacturer.

RNA blots were probed with ³²P-labeled fragments. Prehybridization (1 hour at 42° C.) and hybridization (16 hours at 42° C.) of the membrane filters were carried out in 50% v/v formamide, 1 M NaCl, 1% w/v SDS, 10% w/v dextran sulphate. The membrane filters were generally washed in 2×SSC, 1% w/v SDS at 65° C. for between 1 to 2 hours and then 0.2×SSC, 1% w/v SDS at 65° C. for between 0.5 to 1 hour. Membrane filters were generally exposed to Kodak XAR film with an intensifying screen at −70° C. for 16 to 72 hours.

Abbreviations used in DNA construct preparations are defined in Table 3.

TABLE 3
Abbreviations used in construct preparations

| ABBREVIATION | DESCRIPTION |
|---|---|
| 35S 3′ | terminator fragment from CaMV 35S gene (Franck et al, 1980 supra) |
| 35S 5′ | promoter fragment from CaMV 35S gene (Franck et al, Cell |
| | 21: 285-294, 1980) with an ~60 bp 5′ untranslated leader sequence |
| | from the petunia chlorophyll a/b binding protein gene (Cab 22 |
| | gene) [Harpster et al, MGG, 212: 182-190, 1988] |
| AmCHS 5′ | 1.2 kb promoter fragment from the Antirrhinum majus chalcone |
| | synthase (CHS) gene (Sommer and Saedler, Mol Gen. Gent., |
| | 202: 429-434, 1986) |
| BPF3′5′H#18 | F3′5′H cDNA clone from Viola sp cv Black Pansy (see |
| | International Patent Application No. PCT/AU03/01111) [SEQ ID NO: 1] |
| BPF3′5′H#40 | F3′5′H cDNA clone from Viola sp cv Black Pansy (see |
| | International Patent Application No. PCT/AU03/01111) [SEQ ID NO: 3] |
| BPF3′5′H#40 5′ | ~2.0 kb fragment bearing the promoter region of the Viola |
| | BPF3′5′H#40 gene (SEQ ID NO: 18) |
| CaMV 35S | ~0.2 kb incorporating BglII fragment containing the promoter |
| | region from the Cauliflower Mosaic Virus 35S (CaMV 35S) gene - |
| | (Franck et al, 1980 supra, Guilley et al, Cell, 30: 763-773. 1982) |
| carnANS 3′ | ~0.7 kb fragment containing the terminator region from the |
| | anthocyanidin synthase(ANS) gene of carnation (SEQ ID NO: 11) |
| carnANS 5′ | ~2.5 kb fragment containing the promoter region from the |
| | anthocyanidin synthase (ANS) gene of carnation (SEQ ID NO: 10) |
| ChrysCHS 5′ | ~2.8 kb fragment containing the promoter region from a chalcone |
| | synthase (CHS) gene of chrysanthemum (International Patent |
| | Application No. PCT/AU03/01111) |
| Cineraria | Terminator region from Cineraria F3′5′H gene (SEQ ID NO: 15) |
| F3′5′H 3′ | |
| Cineraria | Promoter region from Cineraria F3′5′H gene (SEQ ID NO: 14) |
| F3′5′H 5′ | |
| Cineraria | ~4.5 kb Cineraria F3′5′H genomic clone with it's own promoter |
| gF3′5′H | and terminator (SEQ ID NO: 12) |
| ds chrysF3′H | “double stranded (ds) chrysanthemum F3′H” fragment harboring a |
| | ~1.0 kb sense partial chrysanthemum F3′H cDNA fragment (see |
| | International PCT application PCT/AU97/00124): 180 bp petunia |
| | DFR-A intron 1 fragment (Beld et al, . Plant Mol. Biol. 13: 491- |
| | 502, 1989): ~1.1 kb antisense partial chrysanthemum F3'H |
| | fragment (see International PCT application PCT/AU97/00124) |
| | with the aim of formation of double stranded (hairpin loop) RNA |
| | molecule to induce silencing of the endogenous chrysanthemum |
| | F3′H through RNAi |
| dschrysF3′H* | “double stranded (ds) chrysanthemum F3′H” harboring a 1.1 kb |
| | sense partial chrysanthemum F3′H cDNA fragment (see |
| | International PCT application PCT/AU97/00124): 0.8 kb antisense |
| | partial chrysanthemum F3′H fragment with the aim of formation |
| | of double stranded (hairpin loop) RNA molecule to induce |
| | silencing of the endogenous chrysanthemum F3′H through RNAi |
| Mac | Hybrid promoter consisting of the promoter from the mannopine |
| | synthase (mas) gene and a CaMV 35S enhancer region (Comai et |
| | al, Plant Mol. Biol. 15: 373-381, 1990) |
| mas 3′ | Terminator region from the mannopine synthase (mas) gene of |
| | A. tumefaciens (Janssen and Gardner, Plant Molecular Biology, |
| | 14: 61-72, 1989) |
| nos 3′ | Terminator region from the nopaline synthase (nos) gene of |
| | A. tumefaciens (Depicker et al., J Mol. and Appl. Genetics 1: 561- |
| | 573, 1982) |
| nptII | Kanamycin-resistance gene (encodes neomycin |
| | phosphotransferase which deactivates aminoglycoside antibiotics |
| | such as kanamycin, neomycin and G418) |
| ocs 3′ | ~1.6 kb terminator fragment from octopine synthase gene of |
| | A. tumefaciens (described in Janssen and Gardner, 1989, supra) |
| PetCHS 5′ | ~0.8 kb promoter fragment from the chalcone synthase- A (CHS) |
| | gene of petunia (Koes, Genes involved in flavonoid biosynthesis |
| | in Petunia hybrida: The chalcone synthase multigene family. PhD |
| | thesis, Vrije Universiteit, Amsterdam, Netherlands, 1988) |
| petD8 3′ | ~0.7 kb terminator region from a phospholipid transfer protein |
| | gene (D8) of Petunia hybrida cv. OGB (Holton, Isolation and |
| | characterization of petal specific genes from Petunia hybrida. PhD |
| | thesis, University of Melbourne, Australia, 1992) |
| petD8 5′ | ~3.2 kb promoter region from a phospholipid transfer protein gene |
| | (D8) of Petunia hybrida (Holton, 1992 supra) |
| petHf1 | Petunia F3′5′H Hf1 cDNA clone (Holton et al, 1993, supra) [SEQ |
| | ID NO: 7] |
| petRT 3′ | Terminator region of a 3RT gene from P. hybrida (Brugliera, |
| | 1994, supra) |
| petRT 5′ | Promoter region of an anthocyanidin-3-glucoside |
| | rhamnosyltransferase (3RT) gene from P. hybrida (Brugliera, |
| | Characterization of floral specific genes isolated from Petunia |
| | hybrida. RMIT, Australia. PhD thesis, 1994) |
| R. rugosa DFR 5′ | ~1.0 kb fragment bearing the promoter region of the Rosa rugosa |
| | DFR gene (SEQ ID NO: 16). |
| R. rugosa F3H 5′ | ~1.2 kb fragment bearing the promoter region of the Rosa rugosa |
| | F3H gene (SEQ ID NO: 17). |
| RoseCHS 5′ | ~2.8 kb fragment containing the promoter region from a CHS gene |
| | of Rosa hybrida (see International Patent Application No. |
| | PCT/AU03/01111) [SEQ ID NO: 9] |
| SalviaF3′5′H#47 | F3′5′H cDNA clone from Salvia sp (see International Patent |
| | Application No. PCT/AU03/01111) [SEQ ID NO: 5] |
| SuRB | Chlorsulfuron-resistance gene [encodes Acetolactate Synthase |
| | (ALS)) with its own terminator from Nicotiana tabacum (Lee et |
| | al, EMBO J. 7: 1241-1248, 1988) |
| SuRB 3′ | terminator fragment from Chlorsulfuron-resistance gene of |
| | Nicotiana tabacum (Lee et al, 1988, supra) |
| tml 3′ | ~2.2 kb fragment containing the terminator region of tumour |
| | morphology large gene from A. tumefaciens (Garfinkel et al, |
| | 1981 supra) |

### Example 1

### Introduction of Chimeric F3′5′H Genes into Chrysanthemum

The pattern of hydroxylation of the B-ring of the anthocyanidin molecule plays a key role in determining petal color. The production of the dihydroflavonol, DHM, leads to the production of the purple/blue delphinidin-based pigments in plants such as petunia. The absence of F3′5′H activity has been correlated with the absence of blue flowers in many plant species such as Rosa (eg. rose), Gerbera (eg. gerbera), Antirrhinum (eg. snapdragon), Dianthus (eg. carnation) and Dendranthema (eg. chrysanthemum).

Based on success in producing delphinidin-based pigments in a mutant petunia line (Holton et al, 1993, supra and International Patent Application No. PCT/AU92/00334), in tobacco flowers (International Patent Application No. PCT/AU92/00334), in carnation flowers (International Patent Application No. PCT/AU96/00296) and in rose flowers (International Patent Application No. PCT/AU03/01111), similar chimeric F3′5′H genes were also introduced into chrysanthemum in order to produce selected or desired delphinidin-based pigments and modify flower color.

Chimeric F3′5′H gene expression cassettes are shown in Table 4.

TABLE 4
Summary of chimeric F3′5′H gene expression cassettes contained
in transformation vector constructs used in the transformation of
chrysanthemums (see Table 3 for an explanation of abbreviations).

| PLASMID | FLAVONOID PATHWAY GENES |
|---|---|
| pCGP90 | Mac 5′: petHf1: mas 3′ |
| pCGP1452 | AmCHS 5′: petHf1: petD8 3′ |
| pCGP1455 | genomic petHf1 |
| pCGP1457 | petD8 5′: petHf1: petD8 3′ |
| pCGP1476 | PetCHS 5′: petHf1: mas 3′ |
| pCGP1616 | petRT 5′: petHf1: nos 3′ |
| pCGP1627 | ChrysCHS 5′: petHf1: nos 3′ |
| pCGP1633 | ChrysCHS 5′: petHf1: petRT3′ |
| pCGP1638 | CaMV 35S: petHf1: ocs 3′ |
| pCGP1860 | RoseCHS 5′: petHf1: nos 3′ |
| pCGP2119 | CaMV 35S: SalviaF3′5′H#47: ocs 3′ |
| pCGP2205 | carnANS 5′: BPF3′5′H#18: carnANS 3′ |
| pCGP2217 | RoseCHS 5′: BPF3′5′H#18: nos 3′ |
| pCGP2610 | CaMV 35S: BPF3′5′H #18: ocs 3′; |
| | CaMV 35S: petDFR: 35S 3′ |
| pCGP2788 | CaMV 35S: BPF3′5′H #40: 35S 3′ |
| pCGP3141 | Cineraria gF3′5′H |
| pCGP3149 | 35S 5′: BPF3′5′H#40: SuRB 3′ |
| pCGP3408 | BPF3′5′H#40 5′: BPF3′5′H#40: nos 3′ |
| pCGP3409 | R. rugosa DFR 5′: BPF3′5′H#40: nos 3′ |
| pCGP3410 | R. rugosa F3H 5′: BPF3′5′H#40: nos 3′ |
| pCGP3424 | RoseCHS 5′: BPF3′5′H#18: nos 3′; |
| | 35S 5′: dschrysF3′H: ocs 3′ |
| pCGP3429 | RoseCHS 5′: BPF3′5′H#18: nos 3′; |
| | RoseCHS 5′: dschrysF3′H*: nos 3′ |
| pCGP3434 | RoseCHS 5′: BPF3′5′H#40: nos 3′ |

### Preparation of Chimeric F3′S′H Gene Constructs

The Transformation Vector pCGP90 (Mac: petHf1: mas 3′)

The transformation vector pCGP90 contains a chimeric petunia F3′5′H (petHf1) gene under the control of a promoter fragment from the mannopine synthase (mas) gene of Agrobacterium and a CaMV 35S enhancer region with a terminator fragment from the mas gene of Agrobacterium (mas 3′). The chimeric petunia F3′S′H cassette is in a tandem orientation with respect to the 35S 5′: SuRB selectable marker gene cassette of the plasmid pWTT2132 (DNAP) [see International Patent Application No. PCT/AU03/01111].)] Construction of the transformation vector pCGP90 is described in Holton et al, 1993 supra.

The Transformation Vector pCGP1452 (AmCHS 5′: petHf1: petD8 3′)

The plasmid pCGP1452 contains a chimeric petunia F3′5′H (petHf1) gene under the control of a promoter fragment from the Antirrhinum majus chalcone synthase gene (CHS) [Sommer and Saedler, 1986, supra] with a terminator fragment from the petunia phospholipid transfer protein (PLTP) gene (petD8 3′) [Holton, 1992, supra]. The chimeric petunia F3′5′H cassette is in a tandem orientation with respect to the 35S 5′: SuRB gene of the plasmid pWTT2132 (DNA Plant Technologies, USA=DNAP)(see International Patent Application No. PCT/AU03/01111). Construction of pCGP1452 is described in International Patent Application No. PCT/AU03/01111.

The Transformation Vector pCGP1455 (Genomic petHf1)

A chimeric genomic petunia F3′5′H Hf1 (genomic petHf1) clone was constructed using various petunia F3′5′H genomic and cDNA clone fragments as described below.

Construction of the Intermediate Plasmid pCGP1352

Initially genomic DNA isolated from the petunia cultivar V23 was completely digested with the restriction endonuclease EcoRV. The digested DNA was size-fractionated using a sucrose density gradient. DNA containing 1 to 3 kb fractions was ligated with an EcoRI adaptor and then ligated with λZAPII EcoRI (Stratagene) and packaged to yield a petunia V23 sub-genomic library following methods as recommended by the manufacturer.

Pfus of the V23 sub-genomic library were screened with ³²P-labeled fragments of an EcoRI-EcoRV fragment from pCGP602 containing the 5′ end of petunia F3′5′H petHf1 cDNA clone representing the N-terminal portion of the protein and containing 5′ non-coding sequences (Holton et al, 1993, supra). One of the hybridising clones was designated as pCGP1352 and contained around 2 kb of promoter region of the petunia F3′5′H gene and 5′ coding sequences representing the N-terminal portion of the protein.

Construction of the Intermediate Plasmids, pCGP1354, pCGP1355 and 1356

The library was also screened with ³²P-labeled fragments of an EcoRI-KpnI fragment from pCGP602 (see International Patent Application No. PCT/AU03/01111) containing the full petunia F3′5′H (petHf1) cDNA clone (Holton et al, 1993, supra) including the 3′-non coding region. One hybridising clone was designated as pCGP1354 and contained the coding region of petunia F3′5′H Hf1 and two introns. Another 2 hybridising clones designated as pCGP1355 and pCGP1356 (both with 1.3 kb inserts) contained sequences representing the C-terminal portion of the protein along with the 3′-non coding region of the petunia F3′5′H Hf1 gene.

Construction of the Intermediate Plasmid pCGP1358

A ˜2.4 kb BstX1 fragment (containing the promoter region of the petunia F3′5′H Hf1 gene and sequence representing the N-terminal portion of the F3′5′H protein) derived from pCGP1352 (described above) was ligated with BstXI fragment (containing the coding region of petunia F3′S′H Hf1 cDNA clone along with the 3′-non coding region) derived from pCGP176 (Holton et al, 1993, supra). The resulting plasmid was designated as pCGP1358.

Construction of the Intermediate Plasmid pCGP1359

A whole genomic petunia F3′5′H Hf1 clone was constructed by ligating the following fragments: an EcoRI (of which the recognition sequence is located in the first intron)-PvuII fragment derived from pCGP1354 (described above), a PvuII-HindIII fragment (containing most of the petunia F3′5′H Hf1 coding region) derived from pCGP176 (Holton et al, 1993, supra) and a HindIII-EcoRI fragment (bearing sequences representing the C-terminal portion of the protein along with 3′-non coding sequences and the cloning vector pBluescript SK− (Stratagene)) of pCGP176 (Holton et al, 1993, supra). The resultant plasmid was designated as pCGP1359.

Construction of the Intermediate Plasmid pCGP649

A 2.5 kb product harboring the genomic Hf1 sequence was amplified using the following primers:

| (SEQ ID NO: 19) | |
|---|---|
| Hfl-5 | 5′ TCA AAA TAT TTA CTT CGG CC 3′ |
| (SEQ ID NO: 20) | |
| Hfl-3 | 5′ TTT CAT TCG ACA TCC TTT GG 3′ |

and genomic DNA isolated from P. hybrida cultivar Th7 as template. The amplified product was cloned into EcoRV ddT-filled site of pBluescript (STRATAGENE) to yield the plasmid pCGP649.
Construction of the Intermediate Plasmid pCGP1360

A fragment (bearing the promoter region and sequences representing the N-terminal portion of the F3′5′H protein) was released upon digestion of the plasmid pCGP1352 (described above) with the restriction endonuclease BstXI. The fragment was purified and ligated with the BstXI ends of the plasmid pCGP649 (bearing the Hf1 genomic sequence amplified using P. hybrida cultivar Th7 as template in a pBluescript SK-backbone vector)(described above) to yield the plasmid pCGP1360.

Construction of the Intermediate Plasmid pCGP1361

An ˜2.5 kb EcoRI fragment of pCGP1360 (bearing the Hf1 promoter region, sequences encoding the N-terminal portion of the protein and a section of the first intron)(described above) was ligated with EcoRI ends of the plasmid pCGP1359 (described above) to yield the plasmid pCGP1361 (containing the full genomic Hf1 sequence).

Construction of the Intermediate Plasmid pCGP1363

A 4.5 kb fragment was released from the plasmid pCGP1361 (described above) upon digestion with the REs Sad and BstBI. The fragment was purified and ligated with the Sad and BstBI ends of the plasmid pCGP1356 (described above) to yield pCGP1363.

Construction of the Transformation Vector pCGP1455

A 6 kb fragment bearing the petunia genomic F3′5′H Hf1 clone was released from the plasmid pCGP1363 upon digestion with the REs ApaI and Sad. The ends were repaired and the fragment was purified and ligated with SmaI ends of the plasmid pWTT2132. Correct insertion the petunia gemonic F3′5′H Hf1 clone in a tandem orientation to the selectable marker gene cassette was confirmed by restriction endonuclease analysis of plasmid DNA isolated from tetracycline-resistant transformants. The resulting plasmid was designated pCGP1455.

The Transformation Vector pCGP1457 (petD8 5′: petHf1: pet D8 3′)

The plasmid pCGP1457 contains a chimeric petunia F3′5′H (petHf1) gene under the control of a promoter fragment from the petunia PLTP gene (petD8 5) with a terminator fragment from the petunia PLTP gene (petD8 3′). The chimeric petunia F3′5′H cassette is in a tandem orientation with respect to the 35S 5′: SuRB gene of the plasmid pWTT2132 (DNAP) [see International Patent Application No. PCT/AU03/01111]. Construction of pCGP1457 is described in International Patent Application No. PCT/AU03/01111.

The Transformation Vector pCGP1476 (PetCHS 5′: petHf1: mas 3′)

The transformation vector pCGP1476 contains a chimeric petunia F3′5′H (petHf1) gene under the control of a promoter fragment from the P. hybrida CHS-A gene (PetCHS 5′) [Koes, 1988 supra] with a terminator fragment from the mannopine synthase gene (mas 3′) of Agrobacterium (Janssen and Gardner, 1989, supra). The chimeric petunia F3′5′H cassette is in a tandem orientation with respect to the 35S 5′: SuRB gene of the plasmid pWTT2132 (DNAP) [see International Patent Application No. PCT/AU03/01111].

Construction of Intermediate Plasmid pCGP669 (PetCHS 5′ Fragment in pBluescript II SK)

An ˜800 bp product bearing the PetCHS 5′ promoter fragment was amplified using the following primers:

| (SEQ ID NO: 21) | |
|---|---|
| CHSA + 34 | 5′ ACGTGACAAGTGTAAGTATC 3′ |
| (SEQ ID NO: 22) | |
| CHSA-782 | 5′ GTTTTCCAAATCTTGACGTG 3′ |

and genomic DNA isolated from P. hybrida cv. V30 as template.

The amplified product was ligated into ddT-tailed pBluescript II SK (STRATAGENE) and sequenced. The plasmid was designated pCGP669.

Construction of Intermediate Plasmid pCGP672 (PetCHS 5′:mas 3′)

The plasmid pCGP669 (described above) was firstly linearized by digestion with the REs EcoRI. After repairing the overhanging ends, the linearized plasmid was digested with the restriction endonuclease HindIII releasing an ˜800 bp fragment bearing the PetCHS 5′ promoter fragment. The fragment was purified and ligated with the XbaI (repaired ends)/HindIII ends of the plasmid pCGP293 (a transformation vector containing Mac 5′: mas 3′ expression cassette) [described in Brugliera et al, 1994 supra]. Correct replacement of the Mac 5′ promoter fragment with the PetCHS 5′ promoter fragment was established by restriction endonuclease analysis of plasmid DNA isolated from gentamycin-resistant transformants. The resulting plasmid containing PetCHS 5′: mas 3′ expression cassette was designated as pCGP672.

Construction of pCGP483 (AmCHS petHf1: petD8 3′ in pBI221 Backbone)

A chimeric petunia F3′5′H gene under the control Antirrhinum majus CHS (AmCHS 5′) promoter with a petunia PLTP terminator (petD8 3′) was constructed by cloning the 1.6 kb BclI/FspI petunia F3′5′H (petHf1) fragment from pCGP601 (described in International Patent Application No. PCT/AU03/01111) between a 1.2 kb Antirrhinum majus CHS gene fragment 5′ to the site of translation initiation (Sommer and Saedler, 1986, supra) and a 0.7 kb SmaI/XhoI PLTP fragment (petD8 3′) from pCGP13ΔBam (Holton, 1992, supra), 3′ to the deduced stop codon. The resulting plasmid in a pBluescribe (STRATAGENE) backbone vector was designated pCGP483.

Construction of Intermediate Plasmid pCGP1471 (PetCHS 5′: petHf1: mas 3)

A 1.6 kb fragment harboring the petHf1 cDNA clone was released from the plasmid pCGP483 (described above) upon digestion with the restriction endonucleases XbaI and BamHI. The fragment was purified and ligated with the XbaI/BamHI ends of the plasmid pCGP672 (described above). Correct insertion of the petHf1 cDNA clone between the AmCHS 5′ promoter fragment and petD8 3′ terminator fragment was established by restriction endonuclease analysis of plasmid DNA isolated from gentamycin-resistant transformants. The resulting plasmid was designated pCGP1471.

Construction of the Transformation Vector pCGP1476 (PetCHS 5′: petHf1: mas 3)

The plasmid pCGP1471 was firstly partially digested with the restriction endonuclease HindIII. The overhanging ends were repaired and the partially digested plasmid was further digested with the restriction endonuclease PstI. The 3.2 kb fragment harboring the PetCHS 5′: petHf1: mas 3′ expression cassette was purified and ligated with SmaI/PstI ends of the Ti plasmid pWTT2132 ((DNAP) [see International Patent Application No. PCT/AU03/01111]. Correct insertion of the expression cassette in a tandem orientation with respect to the 35S 5′: SuRB selectable marker cassette was established by restriction endonuclease analysis of plasmid DNA isolated from tetracycline-resistant transformants. The resulting transformation vector was designated as pCGP1476.

The Transformation Vector pCGP1616 (petRT 5′: petHf1: nos 3′)

The transformation vector pCGP1616 contains a chimeric petunia F3′5′H (petHf1) gene under the control of a promoter fragment from the P. hybrida 3RT gene (petRT 5′) [Brugliera, 1994, supra] with a terminator fragment from the nopaline synthase gene (nos 3) of Agrobacterium (Depicker, et al, 1982, supra). The chimeric petunia F3′5′H cassette is in a tandem orientation with respect to the 35S 5′: SuRB gene of the plasmid pWTT2132 (DNAP) [see International Patent Application No. PCT/AU03/01111]. Construction of pCGP1616 is described in International Patent Application No. PCT/AU03/01111.

The Transformation Vector pCGP1627 (ChrysCHS 5′: petHf1: nos 39

The transformation vector pCGP1627 contains a chimeric petunia F3′5′H (petHf1) gene under the control of a promoter fragment from the chalcone synthase (CHS) gene of chrysanthemum with a terminator fragment from the nopaline synthase (nos) gene of Agrobacterium (nos 3′). The chimeric petunia F3′5′H cassette is in a tandem orientation with respect to the 35S 5′: SuRB selectable marker gene cassette of the plasmid pWTT2132 (DNAP).

### Isolation of Chrysanthemum CHS Promoter

Isolation of a chrysanthemum CHS genomic clone and preparation of the intermediate plasmid pCGP1622 containing the chrysCHS 5′: GUS: nos 3′ expression cassette is described in International Patent Application No. PCT/AU03/01111).

Construction of the Intermediate Plasmid pCGP1624 (chrysCHS 5′: petHf1: nos 39

The petunia F3′5′H (petHf1) cDNA clone was released from the plasmid pCGP1303 (described in International Patent Application No. PCT/AU03/01111) upon digestion with the restriction endonucleases BspHI and XmaI. The 1.6 kb fragment containing the petunia F3′5′H (petHf1) cDNA clone was purified and ligated with NcoI/XmaI ends of pCGP1622. Correct insertion of the fragment was established by restriction endonuclease analysis of plasmid DNA isolated from ampicillin-resistant transformants. The resulting plasmid containing chrysCHS 5′: petHf1: nos 3′ expression cassette was designated as pCGP1624.

Construction of the Transformation Vector pCGP1627

A 4.5 kb fragment containing the chrysCHS 5′: petHf1: nos 3′ gene was released from the plasmid pCGP1624 upon digestion with the restriction endonucleases PstI and BglII. The purified fragment was ligated with PstI/BamHI ends of pWTT2132 (DNAP) [see International Patent Application No. PCT/AU03/01111]. Correct insertion of the chrysCHS 5′: petHf1: nos 3′ gene in a tandem orientation with respect to the 35S 5′: SuRB selectable marker gene cassette was established by restriction endonuclease analysis of plasmid DNA isolated from tetracycline-resistant transformants. The resulting transformation vector was designated pCGP1627.

The Transformation Vector pCGP1633 (ChrysCHS 5′: petHf1: petRT 3′)

The plasmid pCGP1633 contains a chimeric petunia F3′5′H (petHf1) gene under the control of a promoter fragment from the chalcone synthase (CHS) gene of chrysanthemum with a terminator fragment from the 3RT gene (Brugliera, 1994, supra) of Petunia hybrida (petRT 3). The chimeric petunia F3′5′H cassette is in a tandem orientation with respect to the 35S 5′: SuRB selectable marker gene cassette of the plasmid pWTT2132 (DNAP).

Construction of the Intermediate Plasmid pCGP1630 (chrysCHS 5′: petHf1: petRT 3′)

The petRT 3′ terminator fragment was released from the plasmid pCGP1610 (Brugliera, 1994, supra) upon digestion with the restriction endonucleases Sad and BamHI. The 2.5 kb fragment was purified and ligated with SacI/BglII ends of pCGP1624 (described above). Correct insertion of the fragment was established by restriction endonuclease analysis of plasmid DNA isolated from ampicillin-resistant transformants. The resulting plasmid containing chrysCHS 5′: petHf1: petRT 3′ expression cassette was designated as pCGP1630.

Construction of the Transformation Vector pCGP1633 (chrysCHS 5′. petHf1: petRT 3′)

A 6.6 kb fragment containing the chrysCHS 5′: petHf1: petRT 3′ gene was released from the plasmid pCGP1630 upon digestion with the restriction endonuclease PstI. The purified fragment was ligated with PstI ends of pWTT2132 (DNAP) [see International Patent Application No. PCT/AU03/01111]. Correct insertion of the chrysCHS 5′: petHf1 petRT 3′ gene in a tandem orientation with respect to the 35S 5′: SuRB selectable marker gene cassette was established by restriction endonuclease analysis of plasmid DNA isolated from tetracycline-resistant transformants. The plasmid was designated as pCGP1633.

The Transformation Vector pCGP1638 (CaMV 35S: petHf1: ocs 3′)

The transformation vector pCGP1638 contains a chimeric petunia F3′5′H (petHf1) gene under the control of a CaMV 35S promoter (35S 5′) with an octopine synthase terminator (ocs 3′). A ˜60 by 5′ untranslated leader sequence from the petunia chlorophyll a/b binding protein gene (Cab 22 gene) [Harpster et al, 1988, supra] is included between the CaMV 35S promoter fragment and the petunia F3′5′H petHf1 cDNA clone. The chimeric petunia F3′5′H cassette is in a tandem orientation with respect to the 35S 5′: SuRB selectable marker gene cassette of the Ti plasmid vector pWTT2132 (DNAP) [see International Patent Application No. PCT/AU03/01111]. Construction of the transformation vector pCGP1638 is described in International Patent Application No. PCT/AU03/01111.

The Transformation Vector pCGP1860 (RoseCHS 5′: petHf1: nos 39

The transformation vector pCGP1860 contains a chimeric petunia F3′5′H (petHf1) gene under the control of a promoter fragment from the chalcone synthase gene of Rosa hybrida (RoseCHS 5′) with a terminator fragment from the nopaline synthase gene of Agrobacterium (nos 3′). The chimeric petunia F3′5′H cassette is in a tandem orientation with respect to the 35S 5′: SuRB selectable marker gene cassette of the plasmid pWTT2132 (DNAP) [see International Patent Application No. PCT/AU03/01111]. Construction of the transformation vector pCGP1860 is described in International Patent Application No. PCT/AU03/01111.

The Transformation Vector pCGP2119 (CaMV 35S: Salvia F3′5′H: ocs 3′)

The transformation vector pCGP2119 contains a chimeric CaMV 35S: salvia F3′5′H: ocs 3′ gene cassette in a tandem orientation with respect to the 35S 5′: SuRB selectable marker gene cassette of the plasmid pCGP1988 (see International Patent Application No. PCT/AU03/01111). Construction of pCGP2119 is described in International Patent Application No. PCT/AU03/01111.

The Transformation Vector pCGP2205 (carnANS 5′: BPF3′5′H#18: carnANS 3′)

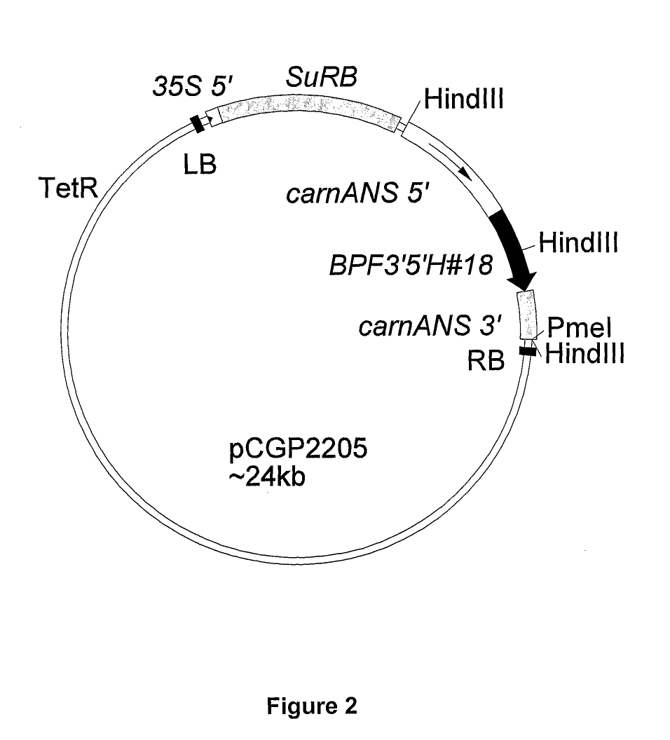

The transformation vector pCGP2205 (FIG. 2) contains a chimeric carnANS 5′: BPF3′5′H#18: carnANS 3′ gene cassette in a tandem orientation with respect to the 35S 5′: SuRB selectable marker gene cassette of the plasmid pCGP1988 (see International Patent Application No. PCT/AU03/01111).

Isolation of Carnation ANS Genomic Clone (pCGP787)

A genomic DNA library was constructed from Dianthus caryophyllus cv. Laguna DNA in EMBL 3 lambda vector (STRATAGENE) using a Sau3A partial digest. The digested genomic DNA was size fractionated on a glycerol density gradient and fractions containing DNA fragments ranging from 15 to 20 kb were pooled and ligated with BamHI ends of EMBL3λ vector. The ligation mixture was packaged using Gigapack XL Gold (PROMEGA). The total size of the library was 1×10⁶ recombinants.

A total of 300,000 pfu of the Laguna genomic library were screened in duplicate with ³²P-labeled fragments of carnation ANS cDNA clone from pCGP786 (see International PCT application PCT/AU96/00296). Hybridization was carried using high stringency conditions (42° C., 50% formamide, 0.1% PVP, 0.1% BSA, 0.1% ficoll, 1% SDS, 100 μg/mL denatured herring sperm DNA. Washes were 2×SSC/1% SDS and 0.2×SSC/1% SDS at 65° C. A genomic clone (4.3) was subsequently purified and further characterised. An 8 kb HindIII fragment of the genomic clone was subcloned into pBluescript KS for further analysis. The resulting plasmid was designated pCGP787.

Construction of the Intermediate Plasmid pCGP1274 (carnANS 5′)

A ˜2.5 kb fragment containing a promoter region from the carnation ANS gene was released from pCGP787 (described above) upon digestion with the restriction endonucleases EcoRI and BamHI. The fragment was purified and ligated with EcoRI/BamHI ends of pBluescript KS to give the plasmid pCGP793. This 2.5 kb fragment also contained 200 bp of 5′ coding sequence of the ANS gene. The 3′ end of the promoter region was then amplified by PCR using primers that introduced an XbaI site 5′ upstream from the ATG codon. The amplified 700 by PCR fragment was then digested with NdeI/XbaI and ligated with NdeI/XbaI ends of pCGP793 to produce a 2.3 kb fragment bearing the ANS promoter without the 200 bp of coding sequence. This new plasmid was designated as pCGP1274.

Construction of the Intermediate Plasmid pCGP1275 (carnANS 5′: carnANS 3′)

The plasmid pCGP795 (described above) was digested with the restriction endonucleases Ecl136II/XbaI to release a 0.7 kb ANS terminator bearing fragment. The fragment was purified and ligated with ApaI (blunted)/XbaI ends of pCGP1274 to produce the plasmid pCGP1275 (containing promoter and terminator fragments of the carnation ANS gene with multicloning sites interrupting the 2 fragments for ease of cloning) [carnANS 5′: carnANS 3′].

Construction of the Intermediate Plasmid pCGP2150 (carnANS 5′: BPF3′5′H#18: carnANS 3)

The plasmid pCGP1959 (containing BPF3′5′H#18 cDNA clone) [described in International Patent Application No. PCT/AU03/01111] was digested with the restriction endonucleases BamHI and Asp718. The ends were repaired and the purified BPF3′5′H#18 fragment was ligated with the repaired PstI/XbaI ends of pCGP1275 (containing carnANS 5′: carnANS 3) to produce the plasmid pCGP2150 (containing the carnANS 5′: BPF3′5′H#18: carnANS 3′ expression cassette).

Construction of the Transformation Vector pCGP2205

The chimeric carnANS 5′: BPF3′5′H#18: carnANS 3′ gene was released from pCGP2150 (described above) upon digestion with the restriction endonuclease ClaI. The ClaI ends of the ˜4.8 kb fragment were repaired and the fragment was isolated, purified and ligated with repaired Asp718 I ends of the plasmid pCGP1988 (see International Patent Application No. PCT/AU03/01111). Correct insertion of carnANS 5′: BPF3′5′H#18: carnANS 3′ gene in a tandem orientation with respect to the 35S 5′: SuRB selectable marker gene cassette was established by restriction endonuclease analysis of plasmid DNA isolated from tetracycline-resistant transformants. The transformation vector was designated as pCGP2205 (FIG. 2).

The Transformation Vector pCGP2217 (Rose CHS: BPF3′5′H#18: nos 3′)

The transformation vector pCGP2217 contains the RoseCHS: Viola F3′5′H#18: nos 3′ expression cassette in a tandem orientation with respect to the 35S 5′: SuRB selectable marker cassette of the Ti plasmid pCGP1988 (see International Patent Application No. PCT/AU03/01111).

### Isolation of Rose CHS Promoter

Isolation of a rose CHS genomic clone and preparation of the plasmids pCGP1116 containing a 2.7-3.0 kb promoter fragment of the CHS gene from rose and pCGP197 containing RoseCHS 5′: GUS: nos 3′ expression cassette in pUC18 backbone is described in International Patent Application No. PCT/AU03/01111.

Construction of Intermediate Plasmid pCGP197 (RoseCHS 5′: GUS: nos 3′ in pUC18 backbone)

An ˜3.0 kb fragment bearing the rose chalcone synthase (CHS) promoter (RoseCHS 5′) was released from the plasmid pCGP1116 upon digestion with the restriction endonucleases HindIII and Asp718. The fragment was purified and ligated with a HindIII/Asp718 fragment from pJB1 ((Bodeau, Molecular and genetic regulation of Bronze-2 and other maize anthocyanin genes. Dissertation, Stanford University, USA, 1994) containing the vector backbone, β-glucoronidase (GUS) and nos 3′ fragments. Correct insertion of the Rose CHS promoter fragment upstream of the GUS coding sequence was established by restriction endonuclease analysis of plasmid DNA isolated from ampicillin-resistant transformants. The resulting plasmid was designated as pCGP197.

Construction of Intermediate Plasmid pCGP2201 (Rose CHS 5′: nos 3′ in pBluescript backbone)

The plasmid pCGP197 (containing RoseCHS 5′: GUS: nos 3′ in pUC18 backbone) was firstly linearized upon digestion with the restriction endonuclease HindIII. The overhanging HindIII ends were repaired and the Rose CHS promoter fragment was then released upon digestion with the restriction endonuclease SalI. The ˜2.8 kb fragment was purified and ligated with XhoI (repaired ends)/SalI ends of the plasmid pCGP1986 (containing the nos terminator fragment in a pBluescript backbone). Correct insertion of the Rose CHS promoter fragment in front of the nos terminator fragment was established by restriction endonuclease analysis of plasmid DNA isolated from ampicillin-resistant transformants. The resulting plasmid was designated as pCGP2201.

Construction of Intermediate Plasmid pCGP2203 (Rose CHS 5′: BPF3′5′H#18: nos 3′ in pBluescript Backbone)

The BPF3′5′H#18 cDNA clone was released from the plasmid pCGP1959 (containing BPF3′5′H#18 in a pBluescript backbone) [described in International Patent Application No. PCT/AU03/01111] upon digestion with the restriction endonucleases BamHI and Asp718. After repairing the overhanging ends, a 1.6 kb fragment containing the BPF3′5′H#18 was purified and ligated with the SalI (repaired ends) of pCGP2201 (described above). Correct insertion of the BPF3′5′H#18 cDNA clone between the Rose CHS 5′ and nos 3′ fragments was established by restriction endonuclease analysis of plasmid DNA isolated from ampicillin-resistant transformants. The resulting plasmid was designated as pCGP2203.

Construction of Transformation Vector pCGP2217 (Rose CHS 5′: BPF3′5′H#18: nos 3′ in pCGP1988 Backbone)

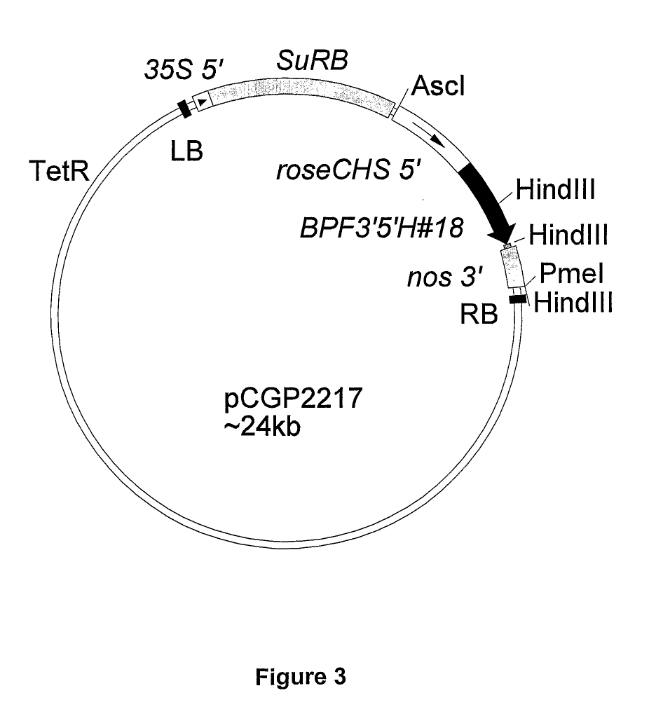

The plasmid pCGP2203 was firstly linearized upon digestion with the restriction endonuclease SpeI. The overhanging ends were repaired and the fragment containing the Rose CHS 5′: BPF3′5′H#18: nos 3′ expression cassette was released upon digestion with the restriction endonuclease BglII. The ˜5 kb fragment was purified and ligated with the SalI (repaired ends)/BamHI ends of the plasmid pCGP1988 (see International Patent Application No. PCT/AU03/01111). Correct insertion of Rose CHS 5′: BPF3′5′H#18: nos 3′ gene in a tandem orientation with respect to the 35S 5′: SuRB selectable marker gene cassette was established by restriction endonuclease analysis of plasmid DNA isolated from tetracycline-resistant transformants. The transformation vector was designated as pCGP2217 (FIG. 3).

The Transformation Vector pCGP2788 (CaMV 35S: BPF3′5′H #40: 35S)

The transformation vector pCGP2788 contains the CaMV 35S: BPF3′5′H#40: 35S 3′ expression cassette in a tandem orientation with respect to the 35S 5′: SuRB selectable marker cassette of the Ti plasmid pCGP1988 (see International Patent Application No. PCT/AU03/01111). Construction of pCGP2788 is described in International Patent Application No. PCT/AU03/00079.

The Transformation Vector pCGP3141 (Cineraria gF3′5′H)

The transformation vector pCGP3141 contains a Cineraria F3′5′H gene (see Japanese Patent Application Number 2008-276029) in a tandem orientation with respect to the 35S 5′: SuRB selectable marker cassette of the Ti plasmid pCGP1988 (see International Patent Application No. PCT/AU03/01111).

### Isolation of the Cineraria F3′5′H Genomic Clone

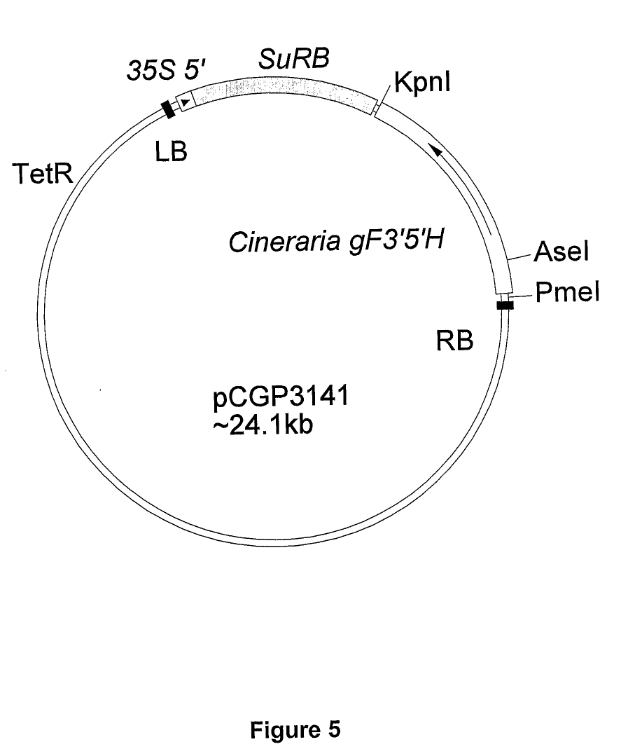

A genomic clone of Cineraria F3′S′H was isolated as described in Japanese Patent Application Number 2008-276029. An ˜4.6 kb fragment harboring the Cineraria gF3′5′H clone (SEQ ID NO: 12) was released from the plasmid gCi01 [Cineraria F3′S′H gene in pBluestar vector backbone (NOVAGEN)] upon digestion with the restriction endonucleases PvuII and EcoRV. The fragment was purified and ligated with the SmaI ends of the plasmid pCGP1988. Correct insertion of the Cineraria gF3′5′H gene in a convergent orientation relative to the 35S 5′: SuRB selectable marker cassette was confirmed by restriction endonuclease analysis of plasmid DNA isolated from tetracycline-resistant transformants. The plasmid was designated as pCGP3141 (FIG. 5).

The Transformation Vector pCGP3149 (35S 5′: BPF3′5′H#40: SuRB 3′)

The transformation vector pCGP3149 contains a 35S 5′: BPF3′5′H#40: SuRB 3′ expression cassette in a tandem orientation with respect to the 35S 5′: SuRB selectable marker cassette of the Ti plasmid pCGP1988 (see International Patent Application No. PCT/AU03/01111).

Fragments used in the construction of the transformation vector pCGP3149 include, a PstI/StuI fragment containing the SuRB 3′ terminator sequence from pCGP1988 (see International Patent Application No. PCT/AU03/01111), a PstI/NcoI fragment bearing the ³⁵S 5′ promoter sequence along with the backbone vector released from the plasmid pCGP1273 (described in International Patent Application No. PCT/AU03/01111) and a BPF3′5′H#40 fragment that originated from the plasmid pCGP1961 (see International Patent Application No. PCT/AU03/01111). The resulting plasmid pCGP3148 contained ³⁵S 5′: BPF3′5′H#40: SuRB 3′ expression cassette in a pBluescript backbone vector.

The 35S 5′: BPF3′5′H#40: SuRB 3′ expression cassette was released from the plasmid pCGP3148 upon digestion with the restriction endonuclease BamHI. The fragment was purified and ligated with th BamHI ends of the plasmid pCGP1988. Correct insertion of the 35S 5′: BPF3′5′H#40: SuRB 3′ chimeric gene in a tandem orientation with respect to the 35S 5′: SuRB selectable marker cassette was confirmed by restriction endonuclease analysis of plasmid DNA isolated from tetracycline-resistant transformants. The plasmid was designated as pCGP3149.

The Transformation Vector pCGP3408 (BPF3′5′H#40 5′: BPF3′5′H#40: nos 3′)

The transformation vector pCGP3409 contains a BPF3′5′H#40 5′: BPF3′5′H#40: nos expression cassette in a tandem orientation with respect to the 35S 5: SuRB selectable marker cassette of the Ti plasmid pCGP1988 (see International Patent Application No. PCT/AU03/01111).

Isolation of the Promoter Fragment from the BPF3′5′H#40 Gene.

Genomic DNA was isolated from the Viola cv. black pansy leaves using DNA easy plant kit (QIAGEN) following procedures recommended by the manufacturer. Three μg of genomic DNA was digested with the restriction endonuclease HindIII. The digested DNA was ligated with HindIII ends of TaKaRa LA PCR (Trade Mark) [contained in the in vitro Cloning Kit TAKARA] using Ligation High (TAKARA) for 40 min at 16° C. using the manufacturer's protocol.

The promoter fragment of BPF3′5H#40 gene was amplified using nested PCR. The 1^{st} PCR was carried out as follows: 4 μL of the ligated genomic DNA (gDNA) [described above] was diluted with 10 μL of water and denatured at 94° C. for 10 min and then transferred to ice. 5 pmol of the following primers:

Primer C1 (5′-GTACATATTGTCGTTAGAACGCGTAATACGACTCA-3′, SEQ ID NO:23) [designed to the cassette and contained in the kit]
Primer BP40-i5 (5′-AGGTGCATGATCGGACCATACTTC-3′, SEQ ID NO:24) designed to the coding region of BPF3′5′H#40 (reverse orientation)
were added to 14 μL ligated gDNA. PCR was carried out in 25 μL following the protocol recommended by the manufacturer (30 cycles of incubation at 98° C. for 20 sec, then 68° C. for 15 min.).

The reaction products of the 1^{st} PCR were then diluted 10-fold with water. 0.5 μL of the diluted 1^{St} PCR products, and 5 pmol of the following primers:

Primer C2 (5′-CGTTAGAACGCGTAATACGACTCACTATAGGGAGA-3′, SEQ ID NO:25) [designed to the cassette and contained in the kit]
Primer BP-i7, (5′-GACCATACTTCTTAGCGAGTTTGGC-3′, SEQ ID NO:26) [designed to the coding region of BPF3′5′H#40 (reverse orientation)]
were subjected to PCR in a total reaction volume of 25 μL. The PCR conditions included a denaturation step at 98° C. for 5 min, followed by 30 cycles of incubation at 98° C. for 20 sec, then 68° C. for 15 min.

The products of the 2^{nd} PCR were cloned into pCR2.1 (INVITROGEN). The inserts were sequenced to confirm that the correct F3′5′H inserts were obtained.

The promoter region of the BPF3′5′H#40 gene along with 200 by of coding sequence was amplified by PCR using 200 ng of the pansy genomic DNA as template along with 50 pmol of the following primers:

Primer BP40-i7 (described above) and
Primer BP40 pro-F (5′-ACTCAAACAAGCATCTCGCCATAGG-3′, SEQ ID NO:27) [designed to the promoter region of BPF3′5′H#40 gene]
in a total reaction volume of 25 μL using Ex-Taq DNA Polymerase (TOYOBO). Conditions used were an initial denaturation step at 98° C. for 5 min followed by 30 cycles of incubation at 98° C. for 20 sec and 68° C. for 15 min.

The PCR products were cloned into the cloning vector pCR2.1 (INVITROGEN). The inserts were sequenced and one plasmid containing 200 bp of coding region along with ˜2.1 kb of 5′ non coding region was designated as pSFL614.

Construction of the Intermediate Plasmid pSFL620 (BPF3′5′H#40 5′:BPF3′5′H#40: nos 3)

About 1.1 kb of the 5′-upstream region of the genomic sequence in pSFL614 (described above) was used to control expression of BPF3′5′H#40 coding sequence as described below. A BamHI site in the region was converted to NheI for convenience of construction.

A 470 bp DNA fragment bearing the 5′ region of the promoter of the BPF3′5′H#40 gene was amplified using 1 ng of the pSFL614 plasmid as template along with 50 pmol of the following primers:

SEQ ID NO: 28)
Primer BP40 pro-HindIII-F
(5′AAG CTT GTG ATC GAC ATC TCT CTC C-3′,
and
SEQ ID NO: 29)
Primer BP40 pro-NheI-R
(5′- CGA GGC TAG CTA AAC ACT TAT -3′,

in a total reaction volume of 25 μL using Ex-Taq DNA Polymerase (TOYOBO).

Conditions used were an initial denaturation step at 98° C. for 5 min followed by 25 cycles of incubation at 98° C. for 20 sec and 68° C. for 15 min. The amplified DNA was cloned into pCR2.1 (INVITROGEN). The sequence was confirmed to be free from PCR error. The 470 by DNA fragment was recovered by digesting with the restriction endonucleases HindIII and NheI (designated as Fragment A).

A 630 by DNA fragment bearing the 3′ region of the promoter of the BPF3′5′H#40 gene was then amplified by PCR using 1 ng of the plasmid pSFL614 (described above) as template along with 50 pmol of the following primers:

SEQ ID NO: 30)
Primer BP40 pro-NheI-F
(5′-TTT AGC TAG CCT CGA AGT TG-3′,
and
SEQ ID NO: 31)
Primer BP40 pro-BamHI-R
(5′- GGA TCC CTA TGT TGA GAA AAA GGG ACT -3′,

in a total reaction volume of 25 μL using Ex-Taq DNA Polymerase (TOYOBO).

Conditions used were an initial denaturation step at 98° C. for 5 min followed by 25 cycles of incubation at 98° C. for 20 sec and 68° C. for 15 min. The amplified DNA was then cloned into pCR2.1 (INVITROGEN). The sequence was confirmed to be free from PCR error. The 630 by DNA fragment was released upon digestion with the restriction endonucleases NheI and BamHI (designated as Fragment B).

The plasmid pSPB567 (described in Patent Publication Number WO2005/017147 and containing enhanced CaMV35S: BPF3′5′H#40: nos 3′ gene cassette in a pUC backbone) was digested with the restriction endonucleases HindIII (partial digestion) and BamHI with the aim of removing the CaMV35S promoter bearing fragment. The 430 bp HindIII/NheI Fragment A (described above and bearing the 5′ region of the promoter of the BPF3′5′H#40 gene) and NheI/BamHI Fragment B (described above and bearing the 3′ region of the promoter of the BPF3′5′H#40 gene) were then ligated with HindIII/BamHI fragment bearing the BPF3′5′H#40: nos 3′ cassette on a pUC backbone from pSPB567 to yield pSFL620 (BPF3′5′H#40 5′: BPF3′5′H#40: nos 3′ in a pUC backbone). The nucleotide sequecne of the BPF3′5′H#40 5′ promoter fragment is shown in SEQ ID No: 18.

Construction of the Intermediate Plasmid pSFL622 (BPF3′5′H#40 5′:BPF3′5′H#40: nos 3′)

The 3.1 kb BPF3′5′H#40 5′: BPF3′5′H#40: nos 3′ expression cassette was excised from the intermediate plasmid pSFL620 (described above) using the restriction endonuclease Pad, and ligated with the PacI ends of pBINPLUS (van Engelen et al, Transgenic Research 4, 288-290, 1995). Correct insertion of the BPF3′5′H#40 5′: BPF3′5′H#40: nos 3′ chimeric gene in a tandem orientation with respect to the nos 5′: nptII: nos 3′ selectable marker gene cassette was established by restriction endonuclease analysis of plasmid DNA isolated from kanamycin-resistant transformants. The resulting plasmid was designated pSFL622.

Construction of the Transformation Vector pCGP3408 (BPF3′5′H#40 5′:BPF3′5′H#40: nos 3′)

The BPF3′5′H#40 5′. BPF3′5′H#40: nos 3′ expression cassette was released from pSFL622 (described above) upon digestion with the restriction endonuclease Pad. The 3.1 kb fragment was purified and ligated with PacI ends of the plasmid pCGP1988 (see International Patent Application No. PCT/AU03/01111). Correct insertion of the BPF3′5′H#40 5′: BPF3′5′H#40: nos 3′ chimeric gene in a tandem orientation with respect to the 35S 5′: SuRB selectable marker gene cassette was established by restriction endonuclease analysis of plasmid DNA isolated from tetracycline-resistant transformants. The resulting transformation vector was designated pCGP3408.

The Transformation Vector pCGP3409 (R. rugosa DFR 5′: BPF3′5′H#40: nos 3′)

The transformation vector pCGP3409 contains a R. rugosa DFR 5′: BPF3′5′H#40: nos expression cassette in a tandem orientation with respect to the 35S 5′: SuRB selectable marker cassette of the Ti plasmid pCGP1988 (see International Patent Application No. PCT/AU03/01111).

Isolation of a DFR Genomic Clone from Rosa rugosa

A Rosa rugosa DFR genomic clone was isolated from a R. rugosa genomic library prepared using λBlueSTART™ Xho I Half-Site Arms Kit (Novagen, http://www.merckbiosciences.com/product/69242).

Genomic DNA was isolated from young leaves of R. rugosa using Nucleon Phytopure [Registered] (TEPNEL Life Sciences). About 100 μg of genomic DNA was partially digested with the restriction endonuclease Sau3AI. The digested ends were partially filled-in with DNA polymerase I Klenow fragment (TOYOBO) in the presence of dGTP and dATP. The genomic DNA (gDNA) was then subjected to size fractionation by sucrose density gradient.

The fractions containing gDNA fragments of around 13 kb were combined and subjected to ethanol precipitation. About 180 ng of the gDNA was ligated with 1 μL of λBlueSTART™ Xho I Half-Site Arms Kit at 4° C. overnight and then packaged. The genomic library obtained was subjected to screening with the rose DFR cDNA (Tanaka et al, Plant and Cell Physiology, 36, 1023-1031, 1995). The DNA labeling and detection were carried out with DIG (Roche Applied Science) following the protocol provided by the manufacturer.

Positive plaques were purified and subjected to in vivo excision following the procedure for λBlueSTAR™ (Novagen). One of the clones (pSFL710) containing an insert of around 10 kb was further characterised. Sequence determination of the DNA fragment in pSFL710 revealed that the fragment contained a sequence homologous to R. hybrida DFR cDNA Tanaka et al, 1995, supra).

In order to isolate the 5′-non coding region of R. rugosa DFR gene, an EcoRI recognition sequence was mutated and HindIII and BamHI recognition sequences were added using PCR. One μL of each of the following primers (both at 25 μM):

| | |
|---|---|
| DFRproHindIIIF | |
| (5′-TAATAAGCTTACAGTGTAATTATC-3′, | SEQ ID NO: 32) |
| and | |
| DFRproNheIR | |
| (5′-TTATGCTAGCGTGTCAAGACCAC-3′, | SEQ ID NO: 33) |

were added to a mix containing the template pSFL710 (described above) and ExTaq DNA Polymerase (TOYOBO) [final volume 50 μL]. PCR conditions included a denaturation step at 94° C. for 5 min followed by 30 cycles of incubation at 94° C. for 30 sec, 50° C. for 30 sec and 72° C. for 30 sec, followed by a final incubation at 72° C. for 7 minutes. A 350 bp DNA fragment was amplified and was designated as Fragment C.

A second PCR was carried out using 1 μL each of the primers (both at 25 μM):

DFRproNheIF

| | |
|---|---|
| (5′-ACACGCTAGCATAAGTCTGTTG -3′, | SEQ ID NO: 34) |
| and | |
| DFRproBamHI-R | |
| (5′-GCTTGGGGATCCATCTTAGG -3′, | SEQ ID NO: 35) |

along with the template pSFL710 (described above) and ExTaq DNA Polymerase (TOYOBO). PCR conditions included a denaturation step at 94° C. for 5 min, followed by 30 cycles of incubation at 94° C. for 30 sec, 50° C. for 30 sec, 72° C. for 30 sec with a final incubation at 72° C. for 7 minutes. A 600 bp DNA fragment was amplified and designated Fragment D.

Fragment C (described above) was digested with the restriction endonuclease HindIII and NheI and Fragment D (described above) was digested with the restriction endonucleases NheI and BamHI. The plasmid pSPB567 (containing enhanced CaMV35S: BPF3′5′H#40: nos 3′ gene cassette in a pUC backbone (described in Patent Publication Number WO2005/017147) was digested with HindIII (partial) and BamHI with the aim of removing the CaMV35S promoter bearing fragment. Therefore HindIII/NheI Fragment C (bearing the 5′ portion of the R. rugosa DFR promoter sequence) along with NheI/BamHI Fragment D (bearing the 3′ portion of the R. rugosa DFR promoter sequence) and HindIII/BamHI pSPB567 (bearing the BPF3′5′H#40: nos 3′ cassette in a pUC backbone) were ligated together to give the plasmid pSFL721 (R. rugosa DFR BPF3′5′H#40: nos 3′ in a pUC based vector). (The sequence of the R. rugosa DFR promoter with an EcoRI recognition site mutated to a NheI site and HindIII and BamHI recognition sites added is shown in SED ID NO:16).

Construction of the Transformation Vector pCGP3409

A 3.11 kb fragment bearing R. rugosa DFR 5′: BPF3′5′H#40: nos 3′ expression cassette was excised from the intermediate plasmid pSFL721 (described above) using the restriction endonuclease Pad, and ligated with PacI ends of the plasmid pCGP1988 (see International Patent Application No. PCT/AU03/01111). Correct insertion of the R. rugosa DFR 5′: BPF3′5′H#40: nos 3′ chimeric gene in a tandem orientation with respect to the 35S 5′: SuRB selectable marker gene cassette was established by restriction endonuclease analysis of plasmid DNA isolated from tetracycline-resistant transformants. The resulting transformation vector was designated pCGP3409.

The Transformation Vector pCGP3410 (R. rugosa F3H 5′: BPF3′5′H#40: nos 3′)

The transformation vector pCGP3410 contains a R. rugosa F3H BPF3′5′H#40: nos 3′ expression cassette in a tandem orientation with respect to the 35S 5′: SuRB selectable marker cassette of the Ti plasmid pCGP1988 (see International Patent Application No. PCT/AU03/01111).

Isolation of a F3H Genomic Clone from Rosa rugosa

The genomic library of Rosa rugosa (described above) was screened with DIG-labeled fragments of the torenia flavanone 3-hydroxylase (F3H) cDNA (NCBI No. AB211958). A selection of hybridising plaques were purified and the plasmids were excised (using methods recommended by the manufacturer). One of the clones, #5, was selected for further study. The plasmid was digested with the restriction endonuclease SpeI and one of the resultant fragment of around 2.6 kb in size was isolated and purified. The fragment was subcloned into SpeI ends of pBluescript SKII-(STRATAGENE) and the resultant plasmid was designated as. Sequence analysis of the fragment contained in pSPB804 revealed that the plasmid contained F3H-like sequence.

In order to amplify a 5′ non coding region of Rosa rugosa F3H gene, PCR was carried out using the following primers:

SEQ ID NO: 36)

| | |
|---|---|
| RrF3H-F | (5′- AAGCTTCTAGTTAGACAAAAAGCTA-3′, |
| and | |

SEQ ID NO: 37)

| | |
|---|---|
| RrF3H | (5′- GGATCCTCTCTTGATATTTCCGTTC-3′,, |

along with 1 ng of pSPB804 as template and the Ex-Taq DNA Polymerase (TOYOBA) in a final reaction volume of 50 μL. PCR conditions included an incubation at 94° C. for 5 min, followed by 30 cycles of incubation at 94° C. for 30 sec, then 50° C. for 30 sec and 72° C. for 30 sec with a final incubation at 72° C. for 7 min. The amplified fragment was subcloned into pCR-TOPO vector (INVITROGEN) to yield the plasmid pSPB811.

The fragment bearing the R. rugosa F3H promoter region (SEQ ID NO:17) was released from pSPB811 upon digestion with the restriction endonucleases HindIII and BamHI. The plasmid pSPB567 (described in Patent Publication Number WO2005/017147 and containing enhanced CaMV35S: BPF3′5′H#40: nos 3′ gene cassette in a pUC backbone) was digested with the restriction endonucleases HindIII (partial digestion) and BamHI with the aim of removing the CaMV35S promoter bearing fragment. The 1.2 kb HindIII/BamHI fragment bearing the R. rugosa F3H promoter region from pSPB811 was then ligated with HindIII/BamHI fragment bearing the BPF3′5′H#40: nos 3′ cassette on a pUC backbone from pSPB567 to yield pSFL814 (R. rugosa F3H 5′: BPF3′5′H#40: nos 3′ in a pUC backbone).

Construction of the Transformation Vector pCGP3410

An ˜3.2 kb fragment bearing the R. rugosa F3H 5′: BPF3′5′H#40: nos 3′ expression cassette was excised from the intermediate plasmid pSFL814 (described above) using the restriction endonucleases PacI and AscI, and ligated with PacI/AscI ends of the plasmid pCGP1988 (see International Patent Application No. PCT/AU03/01111). Correct insertion of the R. rugosa F3H 5′: BPF3′5′H#40: nos 3′ chimeric gene in a tandem orientation with respect to the 35S 5′: SuRB selectable marker gene cassette was established by restriction endonuclease analysis of plasmid DNA isolated from tetracycline-resistant transformants. The resulting transformation vector was designated pCGP3410.

The Transformation Vector pCGP3424 (RoseCHS 5′: BPF3′5′H#18: nos 3′: 35S 5′: ds chrysF3′H: ocs 3′)

The transformation vector pCGP3424 contains the RoseCHS 5′: BPF3′5′H#18: nos 3′ expression cassette in a tandem orientation with respectto 35S 5′: SuRB selectable marker cassette of the transformation vector pCGP2217 (described above) along with a 35S 5′: ds chrysF3′H: ocs 3′ cassette (from pCGP3134, described below) directed at down-regulating expression of the endogenous chrysanthemum F3′H.

Construction of Intermediate Plasmid pCGP1472 (Petunia DFR-A Genomic Clone)

A genomic library was made from Petunia hybrida cv. Old Glory Blue DNA in the vector λ2001 (Holton, 1992 supra). Approximately 200,000 pfu were plated out on NZY plates, lifts were taken onto NEN filters and the filters were hybridized with 400,000 cpm/mL of ³²P-labeled petunia DFR-A cDNA fragment (described in Brugliera et al, The Plant Journal 5(1): 81-92, 1994). Hybridising clones were purified, DNA was isolated from each and mapped by restriction endonuclease digestion. A 13 kb Sad fragment of one of these clones was isolated and ligated with Sad ends of pBluescriptll to create the plasmid pCGP1472. Finer mapping indicated that an ˜5.3 kb BglII fragment contained the entire petunia DFR-A gene (Beld et al, Plant Mol. Biol. 13: 491-502, 1989).

Construction of Intermediate Plasmid pCGP3127 (CaMV 35S: Petunia DFR-A Intron 1: ocs 3′).

A fragment bearing 180 by of the petunia DFR-A intron 1 was amplified by PCR using the plasmid pCGP1472 (described above) as template and the following primers:

(SEQ ID NO: 38)
DFRint F
5′ GCAT CCATGG AGATCT TCG TGA TCC TGG TAT GTT
TG 3′
(SEQ ID NO: 39)
DFRint R
5′ GCAT TCTAGA CTT CTT GTT CTC TAC AAA ATC 3′

The forward primer (DFRint F) was designed to incorporate the restriction endonuclease recognition sites NcoI and BglII at the 5′-end. The reverse primer (DFRint R) was designed to incorporate an XbaI restriction endonuclease recognition site at the 3′-end of the 180 by product that was amplified. The resulting 180 by PCR product was then digested with the restriction endonucleases NcoI and XbaI and ligated with NcoI/XbaI ends of the plasmid pCGP1634 (described in International Patent Application No. PCT/AU03/01111). Correct insertion of the petunia DFR-A intron 1 fragment between the 35S 5′ and ocs 3′ fragments of pCGP1634 was confirmed by restriction endonuclease analysis of plasmid DNA isolated from ampicillin-resistant transformants. The resulting plasmid was designated pCGP3127.

Construction of Intermediate Plasmid pCGP3369 (Partial Chrysanthemum F3′H Clone).

A 1.2 kb fragment containing a partial chrysanthemum F3′H sequence (The sequence of a full-length chrysanthemum F3′H cDNA clone is shown in SEQ ID NO:44, see International PCT application PCT/AU97/00124) was amplified by PCR using a chrysanthemum petal cDNA library (Klondike) as template. A Klondike petal cDNA library was prepared from RNA isolated from floral stages 1 to 3 according to methods described in International PCT application PCT/AU97/00124.

The Following Primers were Used in the PCR:

| | |
|---|---|
| Chrys Red F | |
| 5′ CCTCATCCTCGGGTCAGTAC 3′ | (SEQ ID NO: 40) |
| Chrys Red R | |
| 5′ GCAACTGGACCATTCTCAAGC 3′ | (SEQ ID NO: 41) |

The resulting 1.2 kb PCR product was ligated with the T/A-vector pCR2.1 (INVITROGEN). Correct insertion of the PCR product was confirmed by restriction endonuclease analysis of plasmid DNA isolated from ampicillin-resistant transformants and by sequence analysis. The resulting plasmid was designated as pCGP3369.

Construction of Intermediate Plasmid pCGP3133 (Chrysanthemum F3′H).

The plasmid pCGP3133 contains a partial chrysanthemum F3′H clone generated by PCR to incorporate the XbaI restriction endonuclease recognition site at either ends. An ˜1.1 kb fragment of chrysanthemum F3′H cDNA clone was amplified using the following primers:

| (SEQ ID NO: 42) | |
|---|---|
| 3369Xba.F | 5′GCATCTCTAGAACACCATGGCCTATAGTCG 3′ |
| (SEQ ID NO: 43) | |
| 3369Xba.R | 5′ GCATCTCTAGATTCAAGAGGATCCGCCCAC 3′ |

and the plasmid pCGP3369 (described above) as template. The resulting ˜1.1 kb PCR product was ligated with the T/A-vector pCR2.1 (INVITROGEN). Correct insertion of the PCR fragment into the pCR2.1 vector was confirmed by restriction endonuclease and sequence analysis of ampicillin-resistant transformants. The resulting plasmid was designated pCGP3133.
Construction of Intermediate Plasmid pCGP3129 (35S 5′: Chrysanthemum F3′H Sense: Petunia DFR-A Intron 1: ocs 3′).

An ˜1 kb fragment containing the partial chrysanthemum F3′H cDNA was excised from the intermediate plasmid pCGP3369 (described above) by digestion with the restriction endonucleases NcoI and BamHI. The resulting fragment was purified and ligated with NcoI/BglII ends of the plasmid pCGP3127 (described above). Correct insertion of the chrysanthemum F3′H fragment in a sense direction between the 35S 5′ promoter fragment and the petunia genomic DFR-A intron 1 fragment was confirmed by restriction endonuclease analysis of plasmid DNA isolated from ampicillin-resistant transformants. Further confirmation was obtained by sequence analysis of the correctly mapped transformant. The resulting intermediate plasmid was designated pCGP3129.

Construction of the Intermediate Plasmid pCGP3134 (35S 5′: ds chrysF3′H: ocs 3′).

The intermediate plasmid pCGP3134 contains 35S 5′. ds chrysF3′H: ocs 3′ cassette in a pCR2.1 (INVITROGEN) backbone. The chrysanthemum F3′H partial cDNA fragment was released from the plasmid pCGP3133 (described above) upon digestion with the restriction endonuclease, XbaI. The resulting ˜1.1 kb fragment was purified and ligated with XbaI ends of pCGP3129 (described above). Correct insertion of the chrysanthemum F3′H fragment in an antisense direction between the petunia DFR-A intron 1 and the ocs 3′ terminator fragment was confirmed by restriction endonuclease analysis of plasmid DNA isolated from ampicillin resistant transformants. The resulting plasmid was designated pCGP3134.

Construction of the Transformation Vector pCGP3424 (Rose CHS 5′: BPF3′5′H#18: nos 3′: 35S 5′: ds chrysF3′H: ocs 3′)

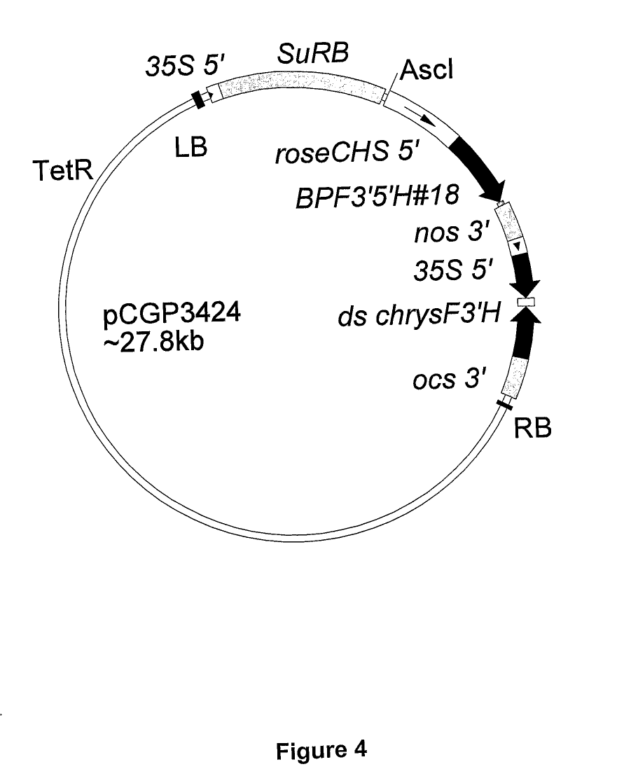

The expression cassette 35S 5′. ds chrysF3′H: ocs 3′ was released from the plasmid pCGP3134 (described above) upon digestion with the restriction endonucleases SmaI and PvuII. The resulting 3.5 kb fragment was purified and ligated with PmeI SAP treated ends of the transformation vector pCGP2217 (described above). Correct insertion of the 35S 5′: ds chrysF3′H: ocs 3′ cassette in a tandem orientation with respect to the RoseCHS BPF3′5′H#18: nos 3′ and 35S 5′: SuRB selectable marker cassette was confirmed by restriction endonuclease analysis of plasmid DNA isolated from tetracycline-resistant transformants. The resulting transformation vector was designated as pCGP3424 (FIG. 4).

The Transformation Vector pCGP3429 (RoseCHS 5′: BPF3′5′H#18: nos 3′: RoseCHS 5′: ds chrysF3′H*: nos 3′)

The transformation vector pCGP3429 contains a RoseCHS 5′: BPF3′5′H#18: nos 3′ expression cassette in a tandem orientation with respect to 35S 5′: SuRB selectable marker cassette of along with a RoseCHS 5′: ds chrysF3′H*: ocs 3′ cassette directed at down-regulating expression of the endogenous chrysanthemum F3′H.

Construction of the Intermediate Plasmid pSPB3349 (RoseCHS 5′ in pBINPlus)

A fragment bearing the RoseCHS 5′ promoter region released from pCGP2203 (described above) upon digestion with the restriction endonucleases HindIII and SmaI digestion. The ˜2.7 kb fragment was purified and ligated with HindIII/SmaI ends of pBINPLus (van Engelen et al., 1995, supra). Correct insertion of the fragment was established by restriction endonuclease analysis of plasmid DNA isolated from kanamycin-resistant transformants. The resulting plasmid was designated pSPB3349.

Construction of the Intermediate Plasmid pSPB3350 (Partial Antisense chrys F3′H: nos 3′)

A fragment bearing a partial region of the chrysanthemum F3′H cDNA clone was released from the plasmid pRm6i (International Patent Application No. PCT/AU97/00124) upon digestion with the restriction endonucleases Sail and HindIII. The 0.83 kb fragment containing 5′-portion of the chrysanthemum F3′H cDNA was purified and ligated with HindIII/SalI ends of the plasmid pSPB176 (pSPB176 contains an e35S 5′: GUS: nos 3′ in pBINPlus and is described in International Patent Application No. PCT/AU03/00079). Correct insertion of the fragment in an antisense direction to the nos 3′ terminator fragment was established by restriction endonuclease analysis of plasmid DNA isolated from kanamycin-resistant transformants. The resulting plasmid was designated pSPB3350.

Construction of the Intermediate Plasmid pSPB3351 (RoseCHS 5′. ds chrys F3′H*: nos 3′)

A fragment bearing the RoseCHS 5′ promoter region was released from the plasmid pSPB3349 (described above) upon digestion with the restriction endonucleases AscI and KpnI. The fragment was purified and designated Fragment A (AscI/KpnI RoseCHS 5′ fragment).

The plasmid pSPB3348 (described above) was firstly linearised with the restriction endonuclease BamHI. The overhanging ends were repaired and then a 1.1 kb fragment bearing the 5′ portion of the chrysanthemum F3′H cDNA clone was released upon digestion with the restriction endonuclease KpnI. The fragment was purified and designated as Fragment B (BamHI(blunted)/KpnI chrysF3′H).

The plasmid pSPB3350 (described above) was firstly linearised with the restriction endonuclease HindIII. The overhanging ends were repaired and then the lineraised plasmid was digested with the restriction endonuclease AscI. The linearised plasmid was designated as Fragment C (HindIII (blunted)/AscI antisense chrysF3′H: nos 3′ in pBINPlus). Fragments A, B and C were ligated. Correct insertion of the fragments to yield RoseCHS 5′: sense chrysF3′H: antisense chrysF3′H: nos 3′ was established by restriction endonuclease analysis of plasmid DNA isolated from kanamycin-resistant transformants. The resulting plasmid was designated pSPB3351 (RoseCHS 5′:ds chrysF3′H*: nos 3′ in a pBINPlus backbone).

Construction of the Intermediate Plasmid pSPB3352 (RoseCHS 5′: ds chrys F3′H*: nos 3; RoseCHS 5′: BPF3′5′H#18: nos 3′; nos 5′: nptII: nos 3′)

A fragment bearing the RoseCHS 5′: BPF3′5′H#18: nos 3′ expression cassette was released from the plasmid pSPB459 (described in International Patent Application No. WO2005-017147)(contains RoseCHS 5′: BPF3′5′H#18: nos 3′ in a pUC based backbone) upon digestion with the restriction endonuclease Pad. The fragment was purified and ligated with PacI ends of the plasmid pSPB3351 (described above). Correct insertion of the fragment bearing the RoseCHS 5′: BPF3′5′H#18: nos 3′ chimeric gene in a tandem orientation with respect to the RoseCHS 5 ds chrysF3′H*: nos 3′ expression cassette was established by restriction endonuclease analysis of plasmid DNA isolated from kanamycin-resistant transformants. The resulting plasmid was designated pSPB3352.

Construction of the Intermediate Plasmid pCGP3426 (RoseCHS 5: ds chrys F3′H*: nos 3′; 35S 5′: SuRB)

The fragment bearing the RoseCHS 5′. ds chrys F3′H*: nos 3′ chimeric gene was released from the plasmid pSPB3352 (described above) upon digestion with the restriction endonucleases AscI and PacI. The fragment was purified and ligated with AscI/PacI ends of the Ti plasmid pCGP1988 ((see International Patent Application No. PCT/AU03/01111)). Correct insertion of the fragment bearing the RoseCHS 5′: ds chrysF3′H*: nos 3′ chimeric gene in a tandem orientation with respect to the 35S 5′: SuRB selectable marker cassette was established by restriction endonuclease analysis of plasmid DNA isolated from teracylcine-resistant transformants. The resulting plasmid was designated pCGP3426.

Construction of the Transformation Vector pCGP3429 (RoseCHS 5′: ds chrys F3′H*: nos 3′; RoseCHS 5′: BPF3′5′H#18: nos 3; 35S 5′: SuRB)

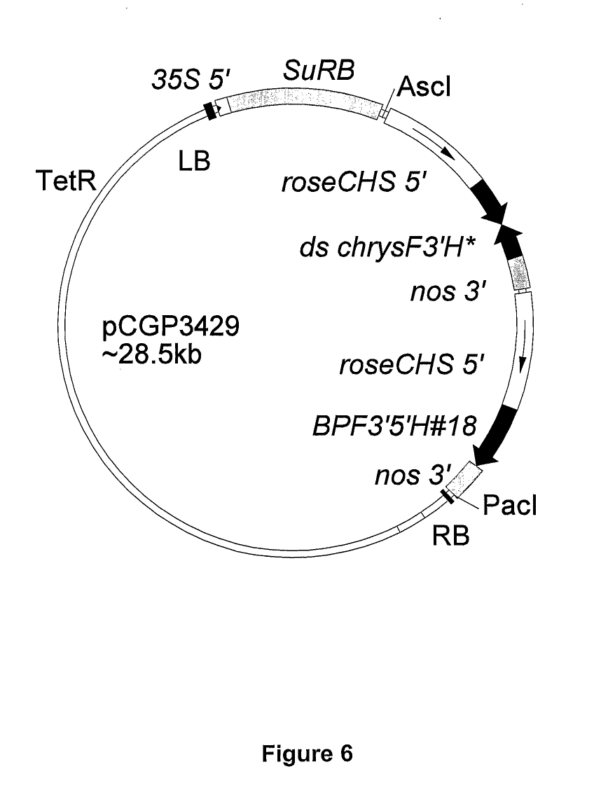

The fragment bearing the RoseCHS 5′: BPF3′5′H#18: nos 3′ chimeric gene was released from the plasmid pSPB3352 (described above) upon digestion with the restriction endonuclease Pad. The fragment was purified and ligated with Pad ends of the plasmid pCGP3426 (described above). Correct insertion of the fragment bearing the RoseCHS 5′: BPF3′5′H#18: nos 3′ in a in a tandem orientation with respect to the RoseCHS 5′: ds chrysF3′H*: nos 3′ chimeric gene and the 35S 5′: SuRB selectable marker cassette was established by restriction endonuclease analysis of plasmid DNA isolated from teracylcine-resistant transformants. The resulting plasmid was designated pCGP3429 (FIG. 6).

The Transformation Vector pCGP3434 (RoseCHS: BPF3′5′H#40: nos)

The transformation vector pCGP3434 contains the RoseCHS 5′: BPF3′5′H#40: nos 3′ expression cassette and the 35S 5′. SuRB selectable marker cassette of the Ti plasmid pCGP1988 (see International Patent Application No. PCT/AU03/01111).

Construction of the Intermediate Plasmid pCGP3425 (RoseCHS 5′: BPF3′5′H#40: nos 3)

A 1.6 kb fragment bearing the BPF3′5′H#40 cDNA clone was excised from the plasmid pCGP1961 (see International Patent Application No. PCT/AU03/01111) using the restriction endonucleases BamHI and Asp718. The ends of the fragment were repaired and then ligated with the repaired ends of SalI of the plasmid pCGP2201 (described above). Correct insertion of the BPF3′5′H#40 cDNA clone between the RoseCHS 5′ and nos 3′ fragments was confirmed by restriction endonuclease analysis of plasmid DNA isolated from ampicillin-resistant transformants. The resulting plasmid was designated as pCGP3425.

Construction of Intermediate Plasmid pCGP3438 (RoseCHS 5′: BPF3′5′H#40: nos 3)

A 4.9 kb fragment bearing the RoseCHS 5′: BPF3′5′H#40: nos 3′ expression cassette was released from the plasmid pCGP3425 (described above) upon digestion with the restriction endonucleases SpeI and BgalII. The fragment was purified and ligated with XbaI/BamHI ends of pUCAP/AscI cloning vector. (The plasmid pUCAP/AscI is a pUC19 based cloning vector with extra cloning sites specifically an AscI recognition site at either ends of the multicloning site.) Correct insertion of the RoseCHS 5′: BPF3′5′H#40: nos 3′ bearing fragment was confirmed by restriction endonuclease analysis of plasmid DNA isolated from ampicillin-resistant transformants. The resulting plasmid was designated as pCGP3438.

Construction of the Transformation Vector pCGP3434 (RoseCHS 5′: BPF3′5′H#40: nos 3′)

A 4.9 kb fragment bearing the RoseCHS 5′: BPF3′5′H#40: nos 3′ expression cassette was released from the plasmid pCGP3438 (described above) upon digestion with the restriction endonuclease AscI. The fragment was purified and ligated with Ascl ends of the plasmid pCGP1988 (see International Patent Application No. PCT/AU03/01111). Correct insertion of the RoseCHS BPF3′5′H#40: nos 3′ cassette in a convergent orientation relative to the 35S 5′: SuRB selectable marker cassette was confirmed by restriction endonuclease analysis of plasmid DNA isolated from tetracycline-resistant transformants. The resulting transformation vector was designated as pCGP3434.

The expression cassettes contained on the T-DNAs from the transformation vectors (Table 4) were introduced into various chrysanthemum cultivars including BlueRidge (BR), Madeline, Moneymaker, Mundial, Improved Reagan, Dark Splendid Reagan. A summary of the results is shown in Table 5.

TABLE 5
Results of analysis of transgenic chrysanthemum petals containing various F3′5′H chimeric genes.

| Cultivar | Construct | Cassettes | #tg | # CC | TLC | HPLC | Northern |
|---|---|---|---|---|---|---|---|
| BR | pCGP1452 | AmCHS 5′: petHf1: petD83′; | 70 | 0 | 0/22 | nd | 4/19 Hf1 |
| | | 35S 5′: SuRB | | | | | 17/18 SuRB |
| Money | pCGP1452 | AmCHS 5′: petHf1: pet D83′ | 217 | 0 | 0/22 | nd | nd |
| maker | | 35S 5′: SuRB | | | | | |
| Mundial | pCGP1452 | AmCHS 5′: petHf1: pet D83′ | 10 | 0 | 0/2 | nd | nd |
| | | 35S 5′: SuRB | | | | | |
| BR | pCGP1476 | PetCHS 5′: petHf1: mas 3′ | 147 | 0 | 0/20 | nd | 1/18 Hf1, |
| | | 35S 5′: SuRB | | | | | 15/15 SuRB |
| Money | pCGP1476 | PetCHS 5′: petHf1: mas 3′; | 19 | 0 | 0/10 | nd | nd |
| maker | | 35S 5′: SuRB | | | | | |
| BR | pCGP 1457 | petD85′: petHf1: petD83′; | 150 | 0 | 0/20 | nd | 1/7 Hf1, |
| | | 35S 5′: SuRB | | | | | 6/7 SuRB |
| BR | pCGP1616 | petRT 5′: petHf1: nos 3′; | 102 | 0 | 0/20 | nd | 0/8 Hf1 |
| | | 35S 5′: SuRB | | | | | |
| Money | pCGP1616 | petRT 5′: petHf1: nos 3′; | 51 | 0 | 0/18 | nd | nd |
| maker | | 35S 5′: SuRB | | | | | |
| BR | pCGP90 | Mac: petHf1: mas 3′; | 32 | 0 | 0/16 | | |
| | | CaMV 35S: nptII: tml3′ | | | | nd | nd |
| Madeline | pCGP90 | Mac: petHf1: mas 3′; | 48 | 0 | 0/33 | nd | 15/15 Hf1 |
| | | CaMV 35S: nptII: tml3′ | | | | | 17/17 nptII |
| BR | pCGP1455 | genomic petHf1; | 62 | 0 | nd | nd | nd |
| | | 35S 5′: SuRB | | | | | |
| BR | pCGP1633 | ChrysCHS 5′: petHf1: petRT3′ | 21 | 0 | nd | nd | nd |
| | | 35S 5′: SuRB | | | | | |
| BR | pCGP1627 | ChrysCHS 5′: petHf1: nos 3′; | 23 | 0 | nd | nd | nd |
| | | 35S 5′: SuRB | | | | | |
| Mundial | pCGP1627 | ChrysCHS 5 ′: petHf1: nos 3′; | 8 | 0 | nd | nd | nd |
| | | 35S 5′: SuRB | | | | | |
| BR | pCGP1638 | 35S 5′: petHf1: ocs 3′; | 19 | 0 | nd | nd | nd |
| | | 35S 5′: SuRB | | | | | |
| BR | pCGP1860 | RoseCHS 5′: petHf1: nos 3′; | 40 | 0 | nd | nd | nd |
| | | 35S 5′: SuRB | | | | | |
| IR | pCGP2119 | CaMV 35S: SalviaF3′5′H#47: | 9 | 0 | 0/8 | nd | 5/5 Salvia |
| | | ocs 3′ | | | | | F3′5′H |
| | | 35S 5′: SuRB | | | | | 5/5 SuRB |
| IR | pCGP2205 | carnANS 5′: BPF3′5′H#18: | 12 | 1 | 9/12* | 3-18% | 4/11 |
| | | carnANS 3′; 35S 5′: SuRB | | | | (8 of 10) | BPF3′5′#18 |
| | | | | | | | 11/11 SuRB |
| DSR | pCGP2205 | carnANS 5′: BPF3′5′H#18: | 19 | 4 | 2 of 2 | 2-22% | nd |
| | | carnANS 3′; | | | | (4 of 4) | |
| | | 35S 5′: SuRB | | | | | |
| IR | pCGP2610 | CaMV 35S: BPF3′5′H #18: ocs | 9 | 0 | 0/6* | 0% | 3/8 BPF3′5′#18 [symbol] |
| | | 3′; | | | | (0 of 2) | 8/8 SuRB |
| | | CaMV 35S: petDFR: 35S 3′; | | | | | 5/8 petDFR |
| | | 35S 5′: SuRB | | | | | |
| IR | pCGP2788 | CaMV 35S: BPF3′5′H #40: 35S | 14 | 0 | 1/14* | 0.5-3% | 3/3 BPF3′5′#40 [symbol] |
| | | 3′; | | | | (2 of 3) | 3/3 SuRB |
| | | 35S 5′: SuRB | | | | | |
| DSR | pCGP2788 | CaMV 35S: BPF3′5′H #40: 35S | 11 | 0 | 1 of 4 | 3% | nd |
| | | 3′; | | | | (1 of 1) | |
| | | 35S 5′: SuRB | | | | | |
| IR | pCGP2217 | Rose CHS 5′: BPF3′5′H#18: nos | 37 | 5 | 25/27* | 0.1-37% | 11/11 |
| | | 3′; | | | | (26 of 27) | BPF3′5′#18 |
| | | 35S 5′: SuRB | | | | | 11/11 SuRB |
| DSR | pCGP2217 | RoseCHS 5′: BPF3′5′H#18: nos | 25 | 2 | 11 of | 12% | nd |
| | | 3′; | | | 16 | (1 of 2) | |
| | | 35S 5′: SuRB | | | | | |
| IR | pCGP3141 | Cineraria gF3′5′H; | 50 | 2 | 8 of 24 | 3-20% | 10/10 cinF3′5′H [symbol] |
| | | 35S 5′: SuRB | | | | (11 of 12) | 10/10 SuRB |
| DSR | pCGP3141 | Cineraria gF3′5′H; | 12 | 1 | 1 of 1 | 7-11% | nd |
| | | 35S 5′: SuRB | | | | (3 of 3) | |
| IR | pCGP3149 | 35S 5′: BP3′5′H#40: SuRB 3′ | 80 | 0 | 5 of 18 | 0.4-9% | 10/10 |
| | | 35S5′: SuRB | | | | (4 of 5) | BPF3′5′H#40 |
| | | | | | | | 10/10 SuRB |
| DSR | pCGP3149 | 35S 5′: BP3′5′H#40: SuRB 3′ | 12 | 0 | 2 of 11 | nd | nd |
| | | 35S 5′: SuRB | | | | | |
| IR | pCGP3424 | RoseCHS 5′: BPF3′5′H#18: nos | 50 | 5 | 4 of 10 | 3-41% | nd |
| | | 3′; | | | | (6 of 6) | |
| | | 35S 5′: dschrysF3′H: ocs | | | | | |
| | | 35S 5′: SuRB | | | | | |
| DSR | pCGP3424 | Rose CHS 5′: BPF3′5′H#18: nos | 30 | 6 | nd | 7-23% | nd |
| | | 3′; | | | | (4 of 4) | |
| | | 35S 5′: dschrysF3′H: ocs 3′ | | | | | |
| | | 35S 5′: SuRB | | | | | |
| IR | pCGP3429 | Rose CHS 5′: BPF3′5′H#18: nos | 2 | 1 | nd | nd | nd |
| | | 3′; | | | | | |
| | | Rose CHS 5′: dschrysF3′H*: nos | | | | | |
| | | 3′ | | | | | |
| | | 35S 5′: SuRB | | | | | |
| IR | pCGP3434 | RoseCHS 5′: BPF3′5′H40: nos | 29 | 0 | nd | nd | nd |
| | | 3′; | | | | | |
| | | 35S 5′: SuRB | | | | | |

Cultivar = The chrysanthemum cultivar that was transformed
Construct = The transformation vector used in the transformation experiment
Cassettes = The gene expression cassettes contained in the transformation vector
#tg = Total number of transgenics produced and flowered to date
# CC = Number of individual transgenic events that produced inflorescences with a significant shift in color compared to that of a non-transformed plant
TLC = Number of inflorescences from individual events in which delphinidin was detected in petals (as determined by TLC) over the total number of individual events analyzed
* = Extracts used for TLC were concentrated (×20) in an effort to detect very low levels of delphinidin
HPLC = Ranges in levels of delphinidin detected by HPLC shown as a percentage of the total anthocyanidins detected in petal extracts. The number of events with delphinidin detected from the total number analysed is represented in brackets
Northern = Number of individual events in which the specific intact F3′5′H, ALS (SuRB) or nptII transcripts were detected by Northern blot analysis in total RNA isolated from petals over the total number of events analyzed
nd = Not done
Hf1 = Petunia F3′5′H Hf1 cDNA clone
cinF3′5′H = 576bp BglII/HindIII cineraria F3′5′H fragment (from SEQ ID No: 12) bearing the 3′ end of the coding region used as a template for labeling [symbol]
indicates data missing or illegible when filed

Surprisingly, of the 23 transformation vector combinations tested to date only five, pCGP2217 (containing Rose CHS 5′: BPF3′5′H#18: nos 3′; 35S 5′: SuRB), pCGP3424 (containing Rose CHS 5′: BPF3′5′H#18: nos 3′; 35S 5′: ds chrysF3′H: ocs 3′; 35S 5′: SuRB), pCGP3429 (containing Rose CHS 5′: BPF3′5′H#18: nos 3′; RoseCHS 5′: ds chrysF3′H*: nos 3′; 35S 5: SuRB), pCGP2205 (containing carnANS 5′: BPF3′5′H#18; carnANS 3′; 35S 5′: SuRB) and pCGP3141 (containing Cineraria gF3′S′H; 35S 5′: SuRB) resulted in a significant change in petal color (Tables 5 and 6). Expression of selected chimeric genes resulted in the accumulation of novel delphinidin-based flavonoid molecules in chrysanthemum petals (Table 5). However only the introduction of the T-DNAs from pCGP2217, pCGP3424, pCGP3429, pCGP2205, pCGP3141 transformation vectors led to high enough levels of delphinidin-based flavonoids to significantly shift the color to the purple and violet ranges.

TABLE 6
Color codes of the transgenic chrysanthemum petals
with significant color changes compared to controls

| | | | RHS Color | RHS Color |
|---|---|---|---|---|
| Cultivar | Construct | Flavonoid gene cassettes | Code | Group |
| IR | Control | — | 75A | PURPLE |
| IR | pCGP2205 | carnANS 5′: BPF3′5′H#18: carnANS 3′ | 84C | VIOLET |
| IR | pCGP2217 | RoseCHS 5′: BPF3′5′H#18: nos 3′ | 84C | VIOLET |
| IR | pCGP3424 | RoseCHS 5′: BPF3′5′H#18: nos 3′; | 84B | VIOLET |
| | | 35S 5′: dschrysF3′H: ocs 3′ | | |
| IR | pCGP3141 | Cineraria gF3′5′H | 77D | PURPLE |
| IR | pCGP3429 | RoseCHS 5′: BPF3′5′H#18: nos 3′; | N82D | PURPLE- |
| | | RoseCHS 5′: dschrysF3′H: nos 3′ | | VIOLET |
| DSR | Control | — | 70B | RED-PURPLE |
| DSR | pCGP2205 | carnANS 5′: BPF3′5′H#18: carnANS 3′ | N78B, 77B, | PURPLE |
| DSR | pCGP2217 | RoseCHS 5′: BPF3′5′H#18: nos 3′ | N78B/C | PURPLE |
| DSR | pCGP3424 | Rose CHS 5′: BPF3′5′H#18: nos 3′; | N80C | PURPLE- |
| | | 35S 5′: dschrysF3′H: ocs 3′ | | VIOLET |
| DSR | pCGP3141 | Cineraria gF3′5′H | 77B | PURPLE |

Cultivar = The chrysanthemum cultivar that was transformed
Construct = The transformation vector used in the transformation experiment
Flavonoid Pathway cassettes = The flavonoid gene expression cassettes contained in the transformation vector construct
RHS Color Code = The color code of the petals from the inflorescences of transgenic chrysanthemums with the most significant color changes as compared to the control petals.
RHS Color Group = The Color group associated with the petal color according to the Royal Horticultural Society, London

The result of the expression of the F3′5′H chimeric genes in the chrysanthemum lines listed in Table 6 shows that there was a shift to purple or purple-violet in petal color as compared to the control color. The petal color of non-transgenic control Improved Reagan (RHSCC 75A) is grouped in the purple color group, however expression of RoseCHS 5′: BPF3′5′H#18: nos 3′ chimeric gene with or without a ds chrysF3′H chimeric gene or carnANS 5′: BPF3′5′H#18: carnANS 3′ gene led to a shift in petal color to the violet range of the RHS Color Charts (see Table 6). The petal color of non-transgenic control Dark Splendid Reagan is in the red-purple color range, however expression of RoseCHS 5′: BPF3′5′H#18: nos 3′ chimeric gene with or without a ds chrysF3′H chimeric gene or carnANS 5′: BPF3′5′H#18:carnANS 3′ or Cineraria gF3′5′H genes led to a shift in petal color to the purple and violet range of the RHS Color Charts.

These results indicate that only a small combination of promoter, terminator and F3′5′H sequences leads to the production of significant delphinidin-based pigments and thus a shift in the petal color to a violet/purple/blue range in chrysanthemum petals. The combination of a F3′5′H sequence from Viola or Cineraria controlled by promoters from flavonoid specific genes such as a CHS promoter from rose or an ANS promoter from carnation or an F3′5′H promoter from Cineraria has led to a significant color change in chrysanthemum petals.

Increased delphinidin production is achieved by down-regulating an endogenous F3′H and/or the endogenous DFR in chrysanthemum using silencing strategies such as co-suppression, antisense or ddRNAi. Where the endogenous DFR is down-regulated a DFR capable of utilizing dihydromyricetin preferentially over dihydroquercetin is included. Such a DFR can be, but is not limited to, be isolated from petunia or iris.

The Rose CHS 5′: BPF3′5′H#18: nos 3′ gene contained in pCGP2217 and pCGP3424 was also introduced into a number of chrysanthemum varieties (“Sei” varieties) including Sei Figaro, Sei Florea, Sei Spire, Sei Titan, Sei Titan406, Sei Norma, Sei Amelie (Table 7).

TABLE 7
Summary of the transgenic
chrysanthemum petals of the “Sei” varieties

| Cultivar | Construct | #tg | #CC | RHSCC | RHSCG |
|---|---|---|---|---|---|
| Sei Figaro | pCGP2217 | 25 | 3 | 76C | PURPLE |
| Sei Figaro | pCGP3424 | 50 | 3 | 76C | PURPLE |
| Sei Figaro | control | | | 65D, 75D, 69B | RED- |
| | | | | | PURPLE |
| Sei Florea | pCGP2217 | 7 | 3 | N80C | PURPLE- |
| | | | | | VIOLET |
| Sei Florea | control | | | 75A, B | PURPLE |
| Sei Spire | pCGP2217 | 42 | 3 | N80D, 76A | PURPLE- |
| | | | | | VIOLET |
| Sei Spire | control | | | 75C, D | PURPLE |
| Sei Titan | pCGP2217 | 50 | 7 | 77C, N80D | PURPLE/ |
| | | | | | PURPLE- |
| | | | | | VIOLET |
| Sei Titan | pCGP3424 | 48 | 4 | N80D, N81B | PURPLE- |
| | | | | | VIOLET |
| Sei Titan | control | | | 78D-, 75B | PURPLE |
| Sei Titan 406 | pCGP2217 | 25 | 4 | 84C- | VIOLET |
| Sei Titan 406 | pCGP3424 | 34 | 3 | 76B | PURPLE |
| Sei Titan 406 | control | | | 75C, D | PURPLE |

Cultivar = The chrysanthemum cultivar that was transformed
Construct = The transformation vector used in the transformation experiment
#tg = Total number of transgenics produced and flowered to date
#CC = Number of individual transgenic events that produced inflorescences with an altered flower color compared to that of a non-transformed plant
RHS Color Code = The color code of the petals from the inflorescences of chrysanthemum with the most significant color changes as compared to the control petals.
RHS Color Group = The Color group associated with the petal color according to the Royal Horticultural Society, London

Expression of the F3′5′H chimeric genes in the chrysanthemum lines listed in Table 7 shows that there was a shift to purple or purple-violet in petal color as compared to the control color. The petal color of non-transgenic control Sei Figaro is in the red-purple color range, however expression of RoseCHS 5′: BPF3′5′H#18: nos 3′ chimeric gene with or without a chrys dsF3′H chimeric gene led to a shift in petal color to the purple range of the RHS Color Charts. The petal color of non-transgenic control Sei Florea is in the purple color range, however expression of RoseCHS 5′: BPF3′5′H#18: nos 3′ chimeric gene in the transgenic lines led to a shift in petal color to the purple-violet range of the RHS Color Charts. The petal color of non-transgenic control Sei Titan is in the purple color range, however expression of RoseCHS: BPF3′5′H#18: nos 3′ chimeric gene with or without a chrys dsF3′H chimeric gene led to a shift in petal color to the purple-violet range of the RHS Color Charts. The petal color of non-transgenic control Sei Titan406 is in the purple color range, however expression of RoseCHS 5′: BPF3′5′H#18: nos 3′ chimeric gene led to a shift in petal color to the violet range of the RHS Color Charts.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Aida et al, Breeding Science, 54: 51-58, 2004
Altschul et al, J. Mol. Biol. 215(3):403-410, 1990
Barker et al, Plant Mol. Biol. 2:235-350, 1983
Beld et al, Plant Mol. Biol. 13:491-502, 1989
Bodeau, Molecular and genetic regulation of Bronze-2 and other maize anthocyanin genes. Dissertation, Stanford University, USA, 1994
Bonner and Laskey, Eur. J. Biochem. 46:83, 1974
Brugliera, Characterization of floral specific genes isolated from Petunia hybrida. RMIT, Australia. PhD thesis, 1994
Brugliera et al, Plant J. 5:81-92, 1994
Bullock et al, Biotechniques 5:376, 1987
Comai et al, Plant Mol. Biol. 15:373-381, 1990
Depicker et al., J Mol. and Appl. Genetics 1:561-573, 1982
da Silva, Biotechnology Advances 21:715-766, 2003.
Dolgov et al, Acta Hort 447:329-334, 1997
Doodeman et al, Theor. Appl. Genet. 67:357-366, 1984
Franck et al, Cell 21:285-294, 1980
Garfinkel et al, Cell 27:143-153, 1981
Glimn-Lacy and Kaufman, Botany Illustrated, Introduction to Plants, Major Groups, Flowering Plant Families, 2^{nd} ed, Springer, USA, 2006
Greve, J. Mol. Appl. Genet. 1:499-511, 1983
Guilley et al, Cell, 30:763-773, 1982
Hanahan, J. Mol. Biol. 166:557, 1983
Harpster et al, MGG, 212:182-190, 1988
Holton, Isolation and characterization of petal specific genes from Petunia hybrida. PhD thesis, University of Melbourne, Australia, 1992
Holton et al, Nature, 366:276-279, 1993
Holton and Cornish, Plant Cell 7:1071-1083, 1995
Huang and Miller, Adv. Appl. Math. 12:373-381, 1991
Inoue et al, Gene 96:23-28, 1990
Janssen and Gardner, Plant Molecular Biology, 14:61-72, 1989
Koes, Genes involved in flavonoid biosynthesis in Petunia hybrida: The chalcone synthase multigene family. PhD thesis, Vrije Universiteit, Amsterdam, Netherlands, 1988
Lazo et al, Bio/technology 9:963-967, 1991
Ledger et al, Plant Cell Reports, 10:195-199, 1991
Lee et al, EMBO J. 7:1241-1248, 1988
Marmur and Doty, J. Mol. Biol. 5:109, 1962
Mol et al, Trends Plant Sci. 3:212-217, 1998
Pearson and Lipman, Proc. Natl. Acad. Sci. USA 85(8):2444-2448, 1988
Plant Molecular Biology Labfax, Croy (ed), Bios scientific Publishers, Oxford, UK, 1993
Plant Molecular Biology Manual (2^{nd} edition), Gelvin and Schilperoot (eds), Kluwer Academic Publisher, The Netherlands, 1994
Salomon et al, EMBO, J. 3:141-146, 1984
Sambrook et al, Molecular Cloning: A Laboratory Manual, Cold Spring Harbor Laboratories, Cold Spring Harbor, N.Y., USA, 1989
Sambrook and Russell, Molecular Cloning: A Laboratory Manual 3^{rd} edition, Cold Spring Harbor Laboratories, Cold Spring Harbor, N.Y., USA, 2001
Schwinn et al, Phytochemistry, 35:145-150, 1993
Seitz and Hinderer, Anthocyanins. In: Cell Culture and Somatic Cell Genetics of Plants. Constabel and Vasil (eds.), Academic Press, New York, USA, 5:49-76, 1988
Sommer and Saedler, Mol. Gen. Gent., 202:429-434, 1986
Strack and Wray, In: The Flavonoids—Advances in Research since 1986. Harbome, J. B. (ed), Chapman and Hall, London, UK, 1-22, 1993
Tanaka et al, Plant and Cell Physiology, 36, 1023-1031, 1995
Tanaka and Mason, In Plant Genetic Engineering, Singh and Jaiwal (eds) SciTech Publishing Llc., USA, 1:361-385, 2003
Tanaka et al, Plant Cell, Tissue and Organ Culture 80:1-24, 2005
Tanaka and Brugliera, In Flowering and Its Manipulation, Annual Plant Reviews Ainsworth (ed), Blackwell Publishing, UK, 20:201-239, 2006
Thompson et al, Nucleic Acids Research 22:4673-4680, 1994
Toguri et al, Plant Biotechnology, 20:121-127, 2003;
Turpen and Griffith, BioTechniques 4:11-15, 1986
van Engelen et al, Transgenic Research 4:288-290, 1995
Wallroth et al, (Mol. Gen. Genet. 202:6-15, 1986
Winkel-Shirley, Plant Physiol. 126:485-493, 2001a
Winkel-Shirley, Plant Physiol. 127:1399-1404, 2001b

1. A genetically modified chrysanthemum plant exhibiting altered inflorescence, said chrysanthemum comprising expressed genetic material encoding a flavonoid 3′,5′ hydroxylase (F3′5′H) enzyme operably linked to a selected promoter and terminator.

2. A genetically modified plant of claim 1 further comprising expressed genetic material which down-regulates an indigenous flavonoid 3′ hydroxylase (F3′H).

3. The genetically modified plant of claim 1 further comprising expressed genetic material which down-regulates an indigenous DFR.

4. The genetically modified plant of claim 1 further comprising expressed genetic material which encodes a heterologous dihydroflavonol-4-reductase (DFR).

5. The genetically modified plant of claim 1 wherein the altered inflorescence is a color in the range red/purple to blue color.

6. The genetically modified plant of claim 5 wherein the color is a violet/purple color.

7. The genetically modified plant of claim 5 wherein the color is selected from the list consisting of 75C, 75D, 76A, 76B, 76C, 77C, 78D, N80D, N81B and 84C of the Royal Horticultural Society color chart.

8. The genetically modified plant of claim 5 wherein the altered inflorescence is in a flower, petal, anther or style of said chrysanthemum plant.

9. The genetically modified plant of claim 1 wherein the chrysanthemum accumulates delphinidin-based flavonoid molecules as determined by TLC or HPLC.

10. The genetically modified plant of claim 1 wherein the chrysanthemum has a genetic background of a chrysanthemum cultivar selected from BlueRidge, Madeline, Moneymaker, Mundial, Improved Reagan, Dark Splendid Reagan, Sei Figaro, Sei Florea, Sei Titan, Sei Titan406, Sei Norma and Sei Amelie.

11. The genetically modified plant of claim 1 wherein the F3′5′H enzyme is from Viola spor Cineraria sp.

12. The genetically modified plant of claim 11 wherein the F3′5′H enzyme is encoded by SEQ ID NO:1, or a nucleotide sequence capable of hybridizing to a complementary form of SEQ ID NO:1 or 3 or 5 or 7 or 12 under low stringency conditions.

13. The genetically modified plant of claim 1 wherein the promoter is from a flavonoid pathway gene.

14. The genetically modified plant of claim 13 wherein the promoter is from a chalione synthase (CHS) or anthocyanidin synthase (ANS) or F3′5′H gene

15. The genetically modified plant of claim 13 wherein the promoter is RoseCHS 5′

16. The genetically modified plant of claim 13 wherein the promoter is carnANS 5′

17. The genetically modified plant of claim 13 wherein the promoter is from a Cineraria gF3′5′H clone

18. The genetically modified plant of claim 13 wherein the promoter is the RoseCHS 5′ promoter and the terminator is nos 3′.

19. The genetically modified plant of claim 1 comprising RoseCHS 5′: BPF3′5′H#18: nos 3′.

20. The genetically modified plant of claim 1 comprising a carnANS 5′ promoter and carnANS 3 terminator.

21. The genetically modified plant of claim 1 comprising carnANS 5′: BPF3′5′H#18: carnANS 3′.

22. The genetically modified plant of claim 1 wherein the promoter and terminator sequences are from the cineraria gF3′5′H clone.

23. The genetically modified plant of claim 1 further comprising genetic material which encodes two or more F3′5′H enzymes and/or DFR enzymes.

24. Progeny, reproductive material, cut flowers, tissue culturable cells and regenerable cells from the genetically modified plant of claim 1.

25. Use of a genetic sequence encoding an F3′5′H enzyme in the manufacture of a chrysanthemum or sports thereof exhibiting altered inflorescence.

26. Use of claim 25 further comprising use of genetic material which inhibits an indigenous F3′H.

27. Use of claim 25 further comprising use of a genetic sequence encoding a heterologous DFR.

28. Use of claim 25 further comprising use of genetic material which inhibits an indigenous DFR.

29. A method for producing a chrysanthemum exhibiting altered inflorescence, the method comprising introducing into regenerable cells of a chrysanthemum plant genetic material encoding an F3′5′H enzyme operably linked to a selected promoter and terminator and regenerating a plant therefrom or selecting genetically modified progeny thereof.

30. The method of claim 29 further comprising the genetic sequence down-regulating an indigenous F3′H.

31. The method of claim 29 further comprising the genetic sequence encoding a heterologous DFR

32. The method of 29 further comprising the genetic sequence down-regulating an indigenous DFR.

33. The method of claim 29 further comprising selecting progeny of the plant having the genetic modification.

34. A genetically modified chrysanthemum plant exhibiting altered inflorescence, said chrysanthemum comprising genetic material encoding an F3′5′H enzyme operably linked to a selected promoter and terminator.

35. The genetically modified plant of claim 34 further comprising genetic material which down-regulates an indigenous F3′H.

36. The genetically modified plant of claim 34 further comprising genetic material which encodes a heterologous DFR.

37. The genetically modified plant of claim 34 further comprising genetic material which down-regulate an indigenous DFR.

38. The genetically modified plant of claim 34 wherein the altered inflorescence is a color in the range of red/purple to blue.

39. The genetically modified plant of claim 38 wherein the color is a violet/purple to blue color.

40. A method for producing a chrysanthemum exhibiting altered inflorescence, said method comprising selecting a chrysanthemum comprising genetic material encoding an F3′5′H enzyme and crossing this plant with another chrysanthemum comprising genetic material encoding another F3′5′H enzyme and then selecting F1 or subsequent generation plants.

41. The method of claim 40 wherein the genetic material in either plant down-regulating an indigenous F3′H.

42. The method of claim 40 wherein the genetic material in either plant encodes a heterologus DFR.

43. The method of claim 40 wherein the genetic material in either plant down-regulates an indigenous DFR.


SEQUENCE LISTING (45 sequences)

SEQ ID NO: 1 - DNA, 1648 bp, Artificial Sequence
  synthetic BPF3′5′H#18.nt, flavonoid 3′,5,-hydroxylase (F3′5′H) cDNA clone from Viola sp. cultivar Black Pansy
agccaatatggcaattccagtcactgaccttgctgtcgcggttatccttttcttgatcactcgcttcctagttcgttctcttttcaagaaaccaaccggaccgctcccgccgggtccttcaggctggcccttggtgggcgcgctccctctcctaggcgccatgcctcacgtcacactagccaacctcgctaaaaaatacggtccgatcatgtacctaaaaatgggcacgtgcgacatggtggtcgcgtccactcccgactcggctcgagccttcctcaaaaccctagacctcaacttctccgaccgcccgcccaacgccggcgccacccatttggcgtacggcgcgcaggacttggtcttcgcgaagtacggtccaaggtggaagaccctaagaaaattgagcaacctccacatgctaggcgggaaggcgctggacgattgggctcacgtgagggctaacgagctaggccacatgcttaacgccatgtgcgaggcgagccggtgcggagagcccgtggtgctggccgagatgctcacgtacgccatggccaacatgatcggtcaagtgatactgagtcggcgcgtgttcgtcaccaaagggacagagtcgaacgagttcaaagatatggtggtcgagttgatgacttccgcggggtatttcaacattggtgacttcataccgtcgattgcttggatggatttgcaagggatcgagcgagggatgaagaaattgcacacgaaattcgatgttttgttgacgaagatgatgaaggagcacagagcgacgagtcatgagcgcgaagggaaatcggatttcctcgacgtcctcttggaagaatgcgagaatacaaatggcgagaagcttaatgttaccaacgtcaaagctgtcctcttgaacttattcacggcgggtacggacacatcttcaagcataatcgaatgggcgttaaccgaaatgatgaagaatccgacgatcttaaaaaagacccaagaagagatggatcgagtcatcggtcgcgatcggagattgctcgaatccgacgtttcgaaactcccgtatttacaagccatagcgaaagaaacatatcgtaaacacccatcgacacctctaaacctgccgaggattgcgatccaagcatgtgaagttgatggctactacatccccaaagacacgaggcttagcgtcaacatttgggcgatcggtcgggacccaagtgtttgggagaatccatcggagttctcgcctgaaagattcttgtctgaggagaatgggaagatcagtccaggcgggaatgattttgagctgattccgtttggagcagggaggagaatttgtgctgggacaaggatgggaatggtccttgtaagttatattttgggcactttggtccattcttttgattggaaattaccaaatggggtcagtgagattaacatggatgagagttttgggcttgcgttgcaaaaggccgtgcctctctcggctacggtcagtccacgattggccccaagcgcgtacgttatatgagctgatgggctgggcctgagcccaaacatattgggtgtgttttatctgtaatttttaatattataaagttcgtaattttgtatttatggttaattatgagttaaaaaaaaaaaaaaaaaa

SEQ ID NO: 2 - protein, 506 aa, Artificial Sequence
  synthetic BPF3′5′H#18.aa, flavonoid 3′,5,-hydroxylase (F3′5′H) deduced amino acid sequence from Viola sp. cultivar Black Pansy
MAIPVTDLAVAVILFLITRFLVRSLFKKPTGPLPPGPSGWPLVGALPLLGAMPHVTLANLAKKYGPIMYLKMGTCDMVVASTPDSARAFLKTLDLNFSDRPPNAGATHLAYGAQDLVFAKYGPRWKTLRKLSNLHMLGGKALDDWAHVRANELGHMLNAMCEASRCGEPVVLAEMLTYAMANMIGQVILSRRVFVTKGTESNEFKDMVVELMTSAGYFNIGDFIPSIAWMDLQGIERGMKKLHTKFDVLLTKMMKEHRATSHEREGKSDFLDVLLEECENTNGEKLNVTNVKAVLLNLFTAGTDTSSSIIEWALTEMMKNPTILKKTQEEMDRVIGRDRRLLESDVSKLPYLQAIAKETYRKHPSTPLNLPRIAIQACEVDGYYIPKDTRLSVNIWAIGRDPSVWENPSEFSPERFLSEENGKISPGGNDFELIPFGAGRRICAGTRMGMVLVSYILGTLVHSFDWKLPNGVSEINMDESFGLALQKAVPLSATVSPRLAPSAYVI

SEQ ID NO: 3 - DNA, 1789 bp, Artificial Sequence
  synthetic BPF3′5′H#40.nt, flavonoid 3′,5,-hydroxylase (F3′5′H) cDNA clone from Viola sp. cultivar Black Pansy
ggcacgaggacaacatggcaattctagtcaccgacttcgttgtcgcggctataattttcttgatcactcggttcttagttcgttctcttttcaagaaaccaacccgaccgctccccccgggtcctctcggttggcccttggtgggcgccctccctctcctaggcgccatgcctcacgtcgcactagccaaactcgctaagaagtatggtccgatcatgcacctaaaaatgggcacgtgcgacatggtggtcgcgtccacccccgagtcggctcgagccttcctcaaaacgctagacctcaacttctccaaccgcccacccaacgcgggcgcatcccacctagcgtacggcgcgcaggacttagtcttcgccaagtacggtccgaggtggaagactttaagaaaattgagcaacctccacatgctaggcgggaaggcgttggatgattgggcaaatgtgagggtcaccgagctaggccacatgcttaaagccatgtgcgaggcgagccggtgcggggagcccgtggtgctggccgagatgctcacgtacgccatggcgaacatgatcggtcaagtgatactcagccggcgcgtgttcgtgaccaaagggaccgagtctaacgagttcaaagacatggtggtcgagttgatgacgtccgccgggtacttcaacatcggtgacttcataccctcgatcgcttggatggatttgcaagggatcgagcgagggatgaagaagctgcacacgaagtttgatgtgttattgacgaagatggtgaaggagcatagagcgacgagtcatgagcgcaaagggaaggcagatttcctcgacgttctcttggaagaatgcgacaatacaaatggggagaagcttagtattaccaatatcaaagctgtccttttgaatctattcacggcgggcacggacacatcttcgagcataatcgaatgggcgttaacggagatgatcaagaatccgacgatcttaaaaaaggcgcaagaggagatggatcgagtcatcggtcgtgatcggaggctgctcgaatcggacatatcgagcctcccgtacctacaagccattgctaaagaaacgtatcgcaaacacccgtcgacgcctctcaacttgccgaggattgcgatccaagcatgtgaagttgatggctactacatccctaaggacgcgaggcttagcgtgaacatttgggcgatcggtcgggacccgaatgtttgggagaatccgttggagttcttgccggaaagattcttgtctgaagagaatgggaagatcaatcccggtgggaatgattttgagctgattccgtttggagccgggaggagaatttgtgcggggacaaggatgggaatggtccttgtaagttatattttgggcactttggtccattcttttgattggaaattaccaaatggtgtcgctgagcttaatatggatgaaagttttgggcttgcattgcaaaaggccgtgccgctctcggccttggtcagcccacggttggcctcaaacgcgtacgcaacctgagctaatgggctgggcctagttttgtgggccttaatttagagacttttgtgttttaaggtgtgtactttattaattgggtgcttaaatgtgtgttttaatttgtatttatggttaattatgactttattgtataattatttatttttcccttctgggtattttatccatttaatttttcttcagaattatgatcatagttatcagaataaaattgaaaataatgaatcggaaaaaaaaaaaaaaaaaaaaaaaaa

SEQ ID NO: 4 - protein, 506 aa, Artificial Sequence
  synthetic BPF3′5′H#40.aa, flavonoid 3′,5,-hydroxylase (F3′5′H) deduced amino acid sequence from Viola sp. cultivar Black Pansy
MAILVTDFVVAAIIFLITRFLVRSLFKKPTRPLPPGPLGWPLVGALPLLGAMPHVALAKLAKKYGPIMHLKMGTCDMVVASTPESARAFLKTLDLNFSNRPPNAGASHLAYGAQDLVFAKYGPRWKTLRKLSNLHMLGGKALDDWANVRVTELGHMLKAMCEASRCGEPVVLAEMLTYAMANMIGQVILSRRVFVTKGTESNEFKDMVVELMTSAGYFNIGDFIPSIAWMDLQGIERGMKKLHTKFDVLLTKMVKEHRATSHERKGKADFLDVLLEECDNTNGEKLSITNIKAVLLNLFTAGTDTSSSIIEWALTEMIKNPTILKKAQEEMDRVIGRDRRLLESDISSLPYLQAIAKETYRKHPSTPLNLPRIAIQACEVDGYYIPKDARLSVNIWAIGRDPNVWENPLEFLPERFLSEENGKINPGGNDFELIPFGAGRRICAGTRMGMVLVSYILGTLVHSFDWKLPNGVAELNMDESFGLALQKAVPLSALVSPRLASNAYAT

SEQ ID NO: 5 - DNA, 1617 bp, Artificial Sequence
  synthetic SalF3′5′H#47.nt, flavonoid 3′,5,-hydroxylase (F3′5′H) cDNA clone from Salvia sp.
agatagtaagcatggaagcccaagaaaatatgttgttgattgctagggcacttgttgtagcatccttactctacattttgatccgtatgtttatctcaaaattgagcacccccggccaccctctgcccccggggccgaggggctttccagtggtgggctcccttcccttgctgggcgacatgccacatgttgccctagcaaaaatggccaaaacttatggcccgatcatgtacttgaaaatgggcacagtcggcatggtcgtggcgtccacgccagacgcggcgcgggcgttcctaaaaacccaggacgctaatttctctaaccggccggtcaacgcgggtgccaccatcctggcatacaatgcccaggacatggtgtttgccccgtacggccccaagtggagattgctgaggaagctgagcagtctccacatgctggggagcaaggccctggaggagtgggccgacgtccggacctcggaggtggggcacatgctggcggcgatgcacgaggccagccgcctggacgaggccgtggggttgccggagatgctggtgtacgcgacggcgaacatgatcgggaaggtgatattgagccggagagttttcgtgacgaaagggaaggagatgaatgagttcaaggaaatggtggtggagctcatgaccacagctggctatttcaacattggtgatttcattccatggcttgcttggatggatttgcaggggattgagagagggatgaagaaactgcacaagaagtgggaccgcttgatcggtaagatgctggatgatcgattgaaatcaacctacaaacgcaacgacaagccagatcttcttgattctctcttggcaaatcatgatgatgagagtaaggatgatgatgaggattgcaagctcaccaccaccaatattaaagcccttttactgaatttatttactgcagggacagacacatcgtcgagcataatagaatgggcactagcggagatgatcaagaatccaagcatccaaaaaagggctcaccaagagatggacagagtcatcgggagagagcggcgtttgctcgaatccgacatcccaaatctgccatacctcaaagccatatgcaaagaggcataccgaaaacacccttccacgccactaaacctgcctcggatctccacggatgcatgcgtcgtcgatggctaccacatccccaagaacacgaggttgagcgtcaacatctgggccataggccgagatcccgacgtttgggagaatccccttgacttcaaccctgacaggtttatgtcagggttgcaggggattgagcccggagggaatcacttcgagctcattccctttggggcggggcgcaggatctgcgccggcagcagaatggggattgtaatagtggagtatttgctggcgacactcgtgcactctttcgaatgggatttgccagccggctcagcggagatggacatggaggaggtgttcgggctggccttgcagaaagctgtaccacttgctgctaggctcactcctaggttgccttcacattgctatgcacctccttctatttaatttgcatatttatatatgttgtgttacattgaaaaaaaaaaaaaaaaaa

SEQ ID NO: 6 - protein, 518 aa, Artificial Sequence
  synthetic SalF3′5′H#47.aa, flavonoid 3′,5,-hydroxylase (F3′5′H) deduced amino acid sequence from Salvia sp.
MEAQENMLLIARALVVASLLYILIRMFISKLSTPGHPLPPGPRGFPVVGSLPLLGDMPHVALAKMAKTYGPIMYLKMGTVGMVVASTPDAARAFLKTQDANFSNRPVNAGATILAYNAQDMVFAPYGPKWRLLRKLSSLHMLGSKALEEWADVRTSEVGHMLAAMHEASRLDEAVGLPEMLVYATANMIGKVILSRRVFVTKGKEMNEFKEMVVELMTTAGYFNIGDFIPWLAWMDLQGIERGMKKLHKKWDRLIGKMLDDRLKSTYKRNDKPDLLDSLLANHDDESKDDDEDCKLTTTNIKALLLNLFTAGTDTSSSIIEWALAEMIKNPSIQKRAHQEMDRVIGRERRLLESDIPNLPYLKAICKEAYRKHPSTPLNLPRISTDACVVDGYHIPKNTRLSVNIWAIGRDPDVWENPLDFNPDRFMSGLQGIEPGGNHFELIPFGAGRRICAGSRMGIVIVEYLLATLVHSFEWDLPAGSAEMDMEEVFGLALQKAVPLAARLTPRLPSHCYAPPSI

SEQ ID NO: 7 - DNA, 1812 bp, Artificial Sequence
  synthetic PetHf1.nt, flavonoid 3′,5,-hydroxylase (F3′5′H) Hf1 cDNA clone from Petunia hybrida
ctttctactagctacttcgttatatatatgtaaaattgtgactttgaaaatcatttaaattatcataaggttcattttatcttgatcaaaatatttacttcggccatatacgttttcctttagtcatgatgctacttactgagcttggtgcagcaacttcaatctttctaatagcacacataatcatttcaactcttatttcaaaaactaccggccggcatctaccgccggggccaagagggtggccggtgatcggagcacttccacttttaggagccatgccacatgtttccttagctaaaatggcaaaaaaatatggagcaatcatgtatctcaaagttggaacatgtggcatggcagttgcttctacccctgatgctgctaaagcattcttgaaaacacttgatatcaacttctccaatcgtccacctaatgcaggtgccactcacttagcttataatgctcaagacatggtttttgcacattatggaccacgatggaagttgctaaggaaattaagcaacttgcatatgctagggggaaaagccttagagaattgggcaaatgttcgtgccaatgagctagggcacatgctaaaatcaatgtccgatatgagtcgagagggccagagggttgtggtggcggagatgttgacatttgccatggccaatatgatcggacaagtgatgctaagcaaaagagtatttgtagataaaggtgttgaggtaaatgaatttaaggacatggttgtagagttaatgacaatagcagggtatttcaacattggtgattttattccttgtttagcttggatggatttacaagggatagaaaaacgaatgaaacgtttacataagaagtttgatgctttattgacaaagatgtttgatgaacacaaagcaactacctatgaacgtaaggggaaaccagattttcttgatgttgttatggaaaatggggacaattctgaaggagaaagactcagtacaaccaacatcaaagcacttttgctgaatttgttcacagctggtacggacacttcttctagtgcaatagaatgggcacttgcagaaatgatgaagaaccctgccattttgaaaaaagcacaagcagaaatggatcaagtcattggaagaaataggcgtttactcgaatccgatatcccaaatctcccttacctccgagcaatttgcaaagaaacatttcgaaaacacccttctacaccattaaatcttcctaggatctcgaacgaaccatgcatagtcgatggttattacataccaaaaaacactaggcttagtgttaacatatgggcaattggaagagatccccaagtttgggaaaatccactagagtttaatcccgaaagattcttgagtggaagaaactccaagattgatcctcgagggaacgattttgaattgataccatttggtgctggacgaagaatttgtgcaggaacaagaatgggaattgtaatggtggaatatatattaggaactttggttcattcatttgattggaaattaccaagtgaagttattgagttgaatatggaagaagcttttggcttagctttgcagaaagctgtccctcttgaagctatggttactccaaggttacaattggatgtttatgtaccatagctatagatgtgtattgtgctataattgcgcatgttgttggttgtagcatgagatattaaaaggagtacatgaagcgcattgcatgagtttaacttgtagctccttaatattttaggtatttttcaattaataagttcttgttggttgggtaaaaaaaaaaaaaaaa

SEQ ID NO: 8 - protein, 506 aa, Artificial Sequence
  synthetic PetHf1.aa, flavonoid 3′,5,-hydroxylase (F3′5′H) Hf1 deduced amino acid sequence from Petunia hybrida
MMLLTELGAATSIFLIAHIIISTLISKTTGRHLPPGPRGWPVIGALPLLGAMPHVSLAKMAKKYGAIMYLKVGTCGMAVASTPDAAKAFLKTLDINFSNRPPNAGATHLAYNAQDMVFAHYGPRWKLLRKLSNLHMLGGKALENWANVRANELGHMLKSMSDMSREGQRVVVAEMLTFAMANMIGQVMLSKRVFVDKGVEVNEFKDMVVELMTIAGYFNIGDFIPCLAWMDLQGIEKRMKRLHKKFDALLTKMFDEHKATTYERKGKPDFLDVVMENGDNSEGERLSTTNIKALLLNLFTAGTDTSSSAIEWALAEMMKNPAILKKAQAEMDQVIGRNRRLLESDIPNLPYLRAICKETFRKHPSTPLNLPRISNEPCIVDGYYIPKNTRLSVNIWAIGRDPQVWENPLEFNPERFLSGRNSKIDPRGNDFELIPFGAGRRICAGTRMGIVMVEYILGTLVHSFDWKLPSEVIELNMEEAFGLALQKAVPLEAMVTPRLQLDVYVP

SEQ ID NO: 9 - DNA, 2934 bp, Artificial Sequence
  synthetic RoseCHS 5′, 2.8kb fragment containing chalcone synthase (CHS) gene promoter region of Rosa hybrida
aagcttcagcaagagttgaagaaatagggacagagccatccatgtgctttgatgaatctgatgggatacaaaatgtgaaagattcacttgctgatttatccagaatttcttcatatagtgaggagaatgttgaaagatctaatgatgagcactctgttaaactagacggaattcatgtgcagcacgagtgtcatgagggcagtgaagaagacaaacctgatggtaagagcggtgagaatgcagttgatctggctaatcatggcatggctcgaactgatttttgtcagataacagaagagattgagaatggagtagtcatcactgagatgagcaacattgccaaccctgataaaactgatattccaaacggggtgcctcaaaatgagactgatgatggatttaataacactcaggatgatgctaatacaaaggaagtgacagaagagaattctgacagacgtgcgaaggaagtgacagaagagaattctgacaaagatgttttgaagaatatccttgaattctcacgtgcttcttctgtggtggattttgaaattccagtgttggatgtgaaatttacttctcttgaaagttgcagtgccacttgttctcttgcagcccttttgtctgaatcgccggaatcaatgactgaagcaccttgtgtgaggcaaattgatgatgtgcccccggttggtgaggagtctagcttgattttggtggaagatcgggagccggttggtcctactcctgatggtaatttttctgtggatatggattactatagtgtagcagaacctttgagcacatgggatgcgaatctgcagtgtgaaacatcaaatagccatgagacttttgctgcaagtctcatttgatagcttctgtgttaataactttgttagtctgtacataaatttgtctagacaagaattggtcgtgtactatcgtgtgtttttgccgtgctttagtactcatgaaccaattcagagaaaactggctgcatattttgaggagtctctgaattcttcaatgctcaactggtatgcatgtaggtggcatatcacttcagggattcttctattctttaactttacgcatcttgacattttgtatataacaaaatcaggtctattgggtgaaagtaattggctagaatggaaagctctacggttttaccgcaggtcaattttcatagctccacaagtgaattgaaaatgctcataggctttatgtttgtcctccacctctggcgacgatgtttgttggggagttaactcaaacctaccaccaaactcgaacccatcttccataatttataatacaaatttgcgatcatttgttcatccaattattgtgacactcggctaccacccaaaatatcggtcacagacccaaacgtattgtcacaacaaatcgtgtctctcgcattaaacacagctagaaagaagagttgaacccacaattcgagcacccactacctatgtacgaagtcatgagttcgagtcaccataggggtagaagtgaaatcatttgatcatctttaaagaaataaaaggaagagttgaacccacaattggctcttgtcccaaaaagaactaatagttcagtgcaccgacgtgtatttgcaccgacataaatggattgttagattatattaaatacactcttaggttattaataaaaatattaattataaatatcaaaagttgagatcatcttataaatgttgggtcagttacaccgtcggtgcatagaataatttccaaactatataatagccttcattttctgatttagctcatgggacatgattgctataaataattgtactcgtagaggcatacttgtgtctttttatacagttgtactgaagctcagaaaagtttatgaaggtgagaactgagaagggcaaggcatttggtagttgaggtatatgagagcatgaaccccatgcattgcagctaccacctctcttttttccttcttcccatacaaataaaaccaactcttctcacctaagtctatcatctttatttatggcagctcttgcttaattagctcatctatattatattatttatctataatatgtgtcactctgtctacctaccagcccaaaataaaactgataatagtcaatttgatgatattttttgttttttgttttgttttgtcttttttgtattgatttttttaaaattaaaatgacttcattttttgtttttgtttttttttctatttttttttatagaaaaattggcaaactttcattatctgttattgatgacaattaagccattaaaacctataattaattatctttcaattcgagtaaatttaaaacggtgtaaaattaaaatatgatcgtattcttaaatgaataaaactcacttaataatagtaatacttgaatcacatctacgaacatagattcttttcatccagtctaaccatgtttgaatatatagagtttgattatggttatgtctttgtccacattttggtttgtaaataaatgtgcaacggaggtatggtactgttgctctatcaaattcaagtttgaattaaaagaaaaaaaaaaagacgatattttgtgcgctttgtttggtaggtaaaacgagagaacaaacgcattccaaatcatgcggattttgatcggcaacacacaccacaaaaaaccgtacacgatgcacgtgccatttgccgggggtttctaacaaggtaattgggcaggcacgtgatcccccagctacccacctctcgcttcccttctcaaactccttttccatgtatatatacaaccccttttctcagaccattatattctaacatttttgctttgctattgtaacgcaacaaaaactgctcattccatccttgttcctccccattttgatcttctctcgacccttctccgagatgggtaccgagctcgaattc

SEQ ID NO: 10 - DNA, 2544 bp, Artificial Sequence
  synthetic carnANS 5′, 2.5kb fragment containing anthocyanidin synthase (ANS) gene promoter region of carnation (Dianthus caryophyllus)
actcgaaatgatactgtgagtctgcgataggctcgttttgaggcggaaattatgattttacgacgtgatcaattgagcatgacttaaatttgcgtcttctcagtcgtcgttgcattgcaatttttagtattttcaggtgctctgaaagtgtttagtacatcgtttttaaaatggatatctttttgttctggtcgacttactcttcgctttttaatgcagacgtgcccgttattgctacgtgtattcacaaaggtatgaccgtgttctgtagcgtctaatgataatatatgaagtcgaggttgcatttgtactagtccgataataattagtatcgttttcatactgatactagatcggaggtcaccatacccgtgaagatttttctgtgagaggaaaagaacccaaggacgaggttcaaatctacacatggaaagacgccacgcttcgtgagttaactgaccttgtatgtcgccatcgcttagcgtagcgctgaacatcgttttcaccctgctccatccatcaaccatttattggtcttatacatgtgtgattgcgttgttcttacatttaggtgaaagacgtttctccagctgctaggagtcgagatgcgaaattgtcgtttgcgactgtataccttgatagaaatggatgcatgcaagtaaagaaggtatcttctaattcatctttcgtagagacatagcgtgaatttggacggggtctttggtttgagaaagataacagctttacgtatttttgtagatgggtgaaaccttttcaaatccgtataagcgtaaagacgacaactgggctttaggggacacattctttcaggtataattgatgcgactaacaatagtctccactgatcatattctactcttctacgttcgatactgactgtttctggttatttggtagacaggagattatttggacgtagcaattcagtagcgtagagatgtttccacacgtgttatcgtaaaagaagcaagataagcctaatgcctagggtggtggtatgacttccgttgcttatcgatcgtgcttgtaagtaatttccgtcttatcttttcctgttatataaagttaatcttctctaggactttcatgaaccttgtttgtgtatttatttctcgatcaacatgatagagctagtttttaagcaacgtatactagtagtctattggaagttaagacacggttcttaaaaaggtacgatccaagtgaagcatgttagatatgacactttcttctagggacgactctcgtatgccacccgactttttcaattttttttgtgaatgttagatgtgtgtatataatgcatccgaaagatgtctcaacgaacaaatgagccacctacttcgatcactcgctatcaatgttattaatgccttgttgattttaatagttgatcaataatagtaaaatctattcaagggtatagtctcccgttcacactcatcggggttacactagcgagctccattaatcggtgccttaatcgagacgctaagaactataccatgacctagtcagcgccatgggactgatgtaggccacacaatctcgatgatccgaaaacgctagagttcaagacctagttcgagaccatggtcacggtttcaaccgcgatatctcaacaatgcaatttttttcgagactagacagacgaataagtcttgtgtacgatgggtagctagtgaattaaaggtaatcactttactcgtgttcacaagaagaccattcatatgaacatttcgtgttctcagacgagctcctttcgctagtttggtaaacttgtttggtgttacctggcttatcatagcccactcaatttggcgaaaacataacaattgtttcacatgcgaaggtctacacacccatactcgtgataaaaacggttgcatttattattattgcctttcgagcaatttacgttgtttgaagtttgtgtaaaaacaaaatcgatctataattactgactggaacggtaatatcaaaactgttaggacctgttcttttcggcttataagagctgaacttataaaagctgtacttataggagctgagctgaactgaacttataagagttgaactgaacttataagggctgaaattaagctgtaaagaacaggttcttatttcgcctgatttgaatccgactaaagttggatgaagatagaggcgagaaacgctcccctttccacagtttgactctactcgaatccgaccaaaattggatgtaaattagaagtgagcattcctcatgccaaccaccgttcttggtcgtgaaaacatgatttgttagtctgctgtatacttcccaaacccgtgataatctgccacacttccaacacttaattgatttttatcaaaattcctacagttttttggttttactcctaaactatgctgtctttttacaagttgttacacctttgtcaacaactttatgctttattatatttcttatataaagaccatataacttccctacactaatgccacaacacttaaaagcatacaacacaaacctcataa

SEQ ID NO: 11 - DNA, 822 bp, Artificial Sequence
  synthetic carnANS 3′, 0.7kb fragment containing anthocyanidin synthase (ANS) gene terminator region of carnation (Dianthus caryophyllus)
actcactacgtgtttacgattgtgtgtttgtcgtgtttgcttaaatcgaccatgtcctcaactttgagaaatcaatagttgtacttttgagttattgttatctcaataacgatattattgcgttatacggagtactgtttagaagacgatatgtaataaatgaaatcgtagttgtctatagcttcatgattatcgttacactattattcttatacttcatctttattttagttaatcttatcaaatacttcgtatattggaaaatttcaaaaagttacttaaaaaataataaaatacaccgaatttcagaaaactcaggaatattcaggcgtaataacatccgttaataccgaaaatattatgaaacgtggcaaatgttggacggtgtgcaatatgagacacaaaaaaacaattgtgaaaaatctttggtcgtaacgaattgcgtcctaaatcatcataataatacaaataaaaaaatgtcattttcattaaatttcttctcttctgtaatcatagaattttattccaacttcatcatctaacttcaggtacatttctccttctctaacttattatcattactttttattgttttaaattatattttttatgtctattaaacgatttaaaatgatcacagtttactctgtacttgtcgtaaataatgtgctgtatgcatacaacagtcttattggtttacgtgaactgttagctgtggttgcatacaatttagattgaaccgacagatatcgcctttactgtcggagggagctgttacagacttacgatactgttcgtaaatggcaagcttgatatcgaatt

SEQ ID NO: 12 - DNA, 4573 bp, Artificial Sequence
  synthetic Cineraria gF3′5′H, 4.5kb Cineraria flavonoid 3′,5,-hydroxylase (F3′5′H) genomic clone with promoter and terminator regions
cagctgcaagtattagtaaaagacgttcggtttgacttttgaggtcaacacatagaaaaattctactccaattttactcgaagtaatgtgattttcaggaaagattacaaagaaactcgtaacatattaaatatgggacaatattagtattaagaacttacccagattcaaatcagtttgaaaatttgaaagttatatataaagataaaatttgacctctcaaggtcaaacagagaaatccaactccgtttatacacaaccttaacgaaattttaagaaaatatcaacgattaccaaaacagttctaacatgttaacacgtggaaacgattcgtctcttgagactaagtaaattatatttacattaatgtgtgattctgaaaaaaggtcgtcaaaatatctattaaatctaatgtacctgtagattatgggcgtgagctcgggttttgagattgggaggcgtttgaatgtagttatctttcacaggaacaacagtgtctttccatggaccaataccgacaacatattctttgatatatttgaagtaacgatctgaagtgatttctgcatacgtctgcaaatgaaaaagaaatcagattataaacatccattgcaaactatccttgcatcgtgtttggatgttcgttttaagcgagtattttatggaatagggagaatcagacaattagttgtaataaaacatgatctttaattgtgctactagtttaagttataatgataatagaaaacatttagtcttcggaaaattatataaattaccaaaaatgggtttaactgtttcaaaccaaaagtggcaagatgtcaggtcggatggattgggtaacgggtcaaaatgggttggactgaaacatgttcaaacatagcgcgtaggccgtagagattacaaaaattctccgttccaaataaggttaacagatatgactatgctgactttttaagtgtcaaatgcgattctcttttccggtatgcataaaaaactgacgacggacattacactatataaaaatttagaaggttataataaaccaagaaaatataattgtattaaattgtgtgagttatatgaattacatagaaccttttatatatggttgaattaccttgctgaacaagaaacctaaacctattagaaatgtctcaaaaatcctaagcttcaggaataccttcccggccttagcgacgaggaagatatgctagagtgtatgtgtgactcgttaaaatcatgaactagaacaaagggaaaggaacaatgttacaatctcaatgattagataggatataactcgataacaaacctaaccagcagagttagatcaagtggtaagtctttgcctttgaagacataggtcgagggttcgatcctcactccatgtggtcggaggtttattggtgaatgcatgcttagctaccgttcaaagtaactttattggtgaatgcatgcttagctaccgttcaaaatcttcaaaaagggtaattatgtctaatatgccatctaagttctaaccaacccttcaaatgttcattcctataattactaaccaattcttacgttgtcaagtaaataaaatgagcattctaaccctaatctgcaccttcatcactggtttgatgttctatgggttggttaatttgcttagccgtcgcgctagccgtcttcctccaggtccaaccccatggccaatcatcggcaacctaatgcaccttggtaaacttccacatcactcgctggcggacttggcgaaaaagtatggtccgttgatacatgtccgactagggtccgttgatgttgtggtggcctcgtctgcgtccgttgctgggcagtttttaaaggtgcatgatgcgaattttgccaacaggccaccaaattctggagctaaacatatggcgtataattatcatgatatggtgtttgcgccgtatggtccaaggtggcgaatgcttcgaaagatgtgctccatgcatctgttttctgccaaagcactcactgattttcgtcaagttcgacaggttttgtactttcactttcgtcatatatatagggagattagtacgagaacgaacacttttaaaatcactttttaataatcaaaatatctttttttttttaaacaaaatcatggaatcttattcaaataacttttctaaccttctaaattttttttaattttttaattttttttttacttacagtgattaagataatcacataaaatatatagataatcacatgaaattttttgtgattatttagttcaaatacattattatcgatatattttttgtgattatcttaaccaccgtaaaaaaaattcaaaaataaaataaaatctgagaaggttaaaaaagttatataaataagattttccgattttgttttcaacaataaaataaaatttcagaacgtaataaaaattgattttttgttaacgagagtttgtaacaatagacggtcaacggaaaatgtgtattatctggtggtatcaccatcggattatgccaagcatgcataaaaaaacaaaatcgtaactacaggaggaggtaacgatactcacgcgcgttttggccaggactggacaatcggcagtgaaactagatcaacaacttaacgtgtgcttcgcaaacacattatcccgaatgatgttagacaggagagtatttggagacggtgatccaaaggcggacgactacaaggatatggtggttgagttgatgactttggccggacaattcaacatcggtgactacattccttggcttgacttgcttgacctacaaggcattgtcaaaaggatgaagaaagttcattctcaattcgattcgttccttgacaccatcattgatgaacatactattggcacgggccgtcatgttgacatgttaagcacaatgatttcactcaaagataatgccgatggagagggagggaagctttcgttcatcgagatcaaagctcttctactggtgcgcgtaatacatagtagtcaacttttttttttttctggtaatgactctttgagcaggtaaaatgtccccaacaggaatcaaacttggtacctatcatttttgggaaaaattttaaaagtactagctttttcaaaaagattatgaaaagtatctgtttttctggacgattgttaaatctaccccaaacgcatgtcttatatgcgttcccttaatcaaacgttgagggtgcgcatatggtacatgcataccctccaaaggagttcccatgcacgttgagggtgcacatatacacatgcgcaccctcttcgtggtttgccaccaaggcaaatcctggaggacagtcaacctttttgatataagttcagatctaactctaggctaatactgttgatgtttcagaacttgttctcagcgggaacggacacgtcatctagtaccgtggaatggggaatagcggaactcattcgccacccacagctaatgaaacaagcgcaagaagaaatggacattgtagttggaaaaaaccggcttgtaacagaaatggacataagccaactaacattccttcaagccattgtgaaagaaacgtttaggctacaccccgcgacgccactttccctgccaaggattgcatcagaaagctgtgaggtcaaggggtatcatgttcctaagggatcgatactctttgttaacgtgtgggccattgctcgacaatcagaattgtggaccgacccacttgaatttcggcctggtcgtttcctaatcccaggagaaaaacctaatgttgaagtgaagccaaatgatttcgaaattgtaccattcgggggaggacgaaggatttgtgcaggtatgagcctcggattgagaatggtcaatttgcttattgcaacattggttcaagcctttgattgggaattggctaatgggttagagccagaaaagcttaacatggaagaagtgtttgggattagccttcaaagggttcaacccttgttggtgcacccgaggccaaggttagcccgtcacgtatacggaacgggttaaggaaataaactgtctgtttgtaagatgaatctgtttgaatttatgtattaaatagttatgctaagaactatttttacaaataaaagtatattggtttgattgttctcgcttagcctttgctaaatcttagatagatgagttgtataacacatcatcattaactcacatcacgtggtaacgatttgtttttgagttaaaatttttaaagaaaggaaagaaagagaaagtaaatataaaaaaatttgtgttcccgagaagttttttacgaaggaagaggggagaaagagagagaattttagagaaattttgagtattttacaacaaaaatcatcctctcatttttgggatgatttggaggatcttttttctttcttttccttcgtccacttcacctccctttctttccaaaaaaatctcggaaacatagcgtaatgataaacaaaaaccaataaaaatgagcaggagcaaaccctagaaggacgaaatcttgaaaatttattctaagatttttaaaaaaaacttggcagttggaaagggcggcggat

SEQ ID NO: 13 - protein, 504 aa, Artificial Sequence
  synthetic Cineraria gF3′5′H, 4.5kb Cineraria flavonoid 3′,5,-hydroxylase (F3′5′H) genomic clone deduced amino acid sequence
MSILTLICTFITGLMFYGLVNLLSRRASRLPPGPTPWPIIGNLMHLGKLPHHSLADLAKKYGPLIHVRLGSVDVVVASSASVAGQFLKVHDANFANRPPNSGAKHMAYNYHDMVFAPYGPRWRMLRKMCSMHLFSAKALTDFRQVRQEEVTILTRVLARTGQSAVKLDQQLNVCFANTLSRMMLDRRVFGDGDPKADDYKDMVVELMTLAGQFNIGDYIPWLDLLDLQGIVKRMKKVHSQFDSFLDTIIDEHTIGTGRHVDMLSTMISLKDNADGEGGKLSFIEIKALLLNLFSAGTDTSSSTVEWGIAELIRHPQLMKQAQEEMDIVVGKNRLVTEMDISQLTFLQAIVKETFRLHPATPLSLPRIASESCEVKGYHVPKGSILFVNVWAIARQSELWTDPLEFRPGRFLIPGEKPNVEVKPNDFEIVPFGGGRRICAGMSLGLRMVNLLIATLVQAFDWELANGLEPEKLNMEEVFGISLQRVQPLLVHPRPRLARHVYGTG

SEQ ID NO: 14 - DNA, 1586 bp, Artificial Sequence
  synthetic Cineraria F3′5′H 5′, Cineraria flavonoid 3′,5,-hydroxylase (F3′5′H) gene promoter region
cagctgcaagtattagtaaaagacgttcggtttgacttttgaggtcaacacatagaaaaattctactccaattttactcgaagtaatgtgattttcaggaaagattacaaagaaactcgtaacatattaaatatgggacaatattagtattaagaacttacccagattcaaatcagtttgaaaatttgaaagttatatataaagataaaatttgacctctcaaggtcaaacagagaaatccaactccgtttatacacaaccttaacgaaattttaagaaaatatcaacgattaccaaaacagttctaacatgttaacacgtggaaacgattcgtctcttgagactaagtaaattatatttacattaatgtgtgattctgaaaaaaggtcgtcaaaatatctattaaatctaatgtacctgtagattatgggcgtgagctcgggttttgagattgggaggcgtttgaatgtagttatctttcacaggaacaacagtgtctttccatggaccaataccgacaacatattctttgatatatttgaagtaacgatctgaagtgatttctgcatacgtctgcaaatgaaaaagaaatcagattataaacatccattgcaaactatccttgcatcgtgtttggatgttcgttttaagcgagtattttatggaatagggagaatcagacaattagttgtaataaaacatgatctttaattgtgctactagtttaagttataatgataatagaaaacatttagtcttcggaaaattatataaattaccaaaaatgggtttaactgtttcaaaccaaaagtggcaagatgtcaggtcggatggattgggtaacgggtcaaaatgggttggactgaaacatgttcaaacatagcgcgtaggccgtagagattacaaaaattctccgttccaaataaggttaacagatatgactatgctgactttttaagtgtcaaatgcgattctcttttccggtatgcataaaaaactgacgacggacattacactatataaaaatttagaaggttataataaaccaagaaaatataattgtattaaattgtgtgagttatatgaattacatagaaccttttatatatggttgaattaccttgctgaacaagaaacctaaacctattagaaatgtctcaaaaatcctaagcttcaggaataccttcccggccttagcgacgaggaagatatgctagagtgtatgtgtgactcgttaaaatcatgaactagaacaaagggaaaggaacaatgttacaatctcaatgattagataggatataactcgataacaaacctaaccagcagagttagatcaagtggtaagtctttgcctttgaagacataggtcgagggttcgatcctcactccatgtggtcggaggtttattggtgaatgcatgcttagctaccgttcaaagtaactttattggtgaatgcatgcttagctaccgttcaaaatcttcaaaaagggtaattatgtctaatatgccatctaagttctaaccaacccttcaaatgttcattcctataattactaaccaattcttacgttgtcaagtaaataaa

SEQ ID NO: 15 - DNA, 545 bp, Artificial Sequence
  synthetic Cineraria F3′5′H 3′, Cineraria flavonoid 3′,5,-hydroxylase (F3′5′H) gene terminator region
ggaaataaactgtctgtttgtaagatgaatctgtttgaatttatgtattaaatagttatgctaagaactatttttacaaataaaagtatattggtttgattgttctcgcttagcctttgctaaatcttagatagatgagttgtataacacatcatcattaactcacatcacgtggtaacgatttgtttttgagttaaaatttttaaagaaaggaaagaaagagaaagtaaatataaaaaaatttgtgttcccgagaagttttttacgaaggaagaggggagaaagagagagaattttagagaaattttgagtattttacaacaaaaatcatcctctcatttttgggatgatttggaggatcttttttctttcttttccttcgtccacttcacctccctttctttccaaaaaaatctcggaaacatagcgtaatgataaacaaaaaccaataaaaatgagcaggagcaaaccctagaaggacgaaatcttgaaaatttattctaagatttttaaaaaaaacttggcagttggaaagggcggcggat

SEQ ID NO: 16 - DNA, 1000 bp, Artificial Sequence
  synthetic R. rugosa DFR 5′, 1.0 kb fragment containing Rosa rugosa dihydroflavonol-4-reductase (DFR) gene promoter region
aagcttacagtgtaattatcttctggatctacacacaaatccaacacttgcatttcaaataataggcataggcaaatcccatcatttgcttctggaagtttgtgaccaaaaagcccaactctaatgaatatgtttgcaactttgcaatagaactttccctttagggaatagtattatgtagtcatcattctttctccctaaaactggttgttgagatgactaaaattcttgacttttaagtggtaggtggtattgcatggattaggtaatggattaatgggttgcagctcacaaaattagagagaagaaagaaatcgaaacctaatacgaagtcaatggtggtcttgacacgctagcataagtctgttgtttaaaaaaaaaaaaaaaaaaaaactgaaactggtcctaaatcctaagcgtgttaggaacaatattttggctaatgatattatcacagtctctcaaaaactgtctcataggtgagtagctctgctgccgaaatcgaaatatctgattaagttggtaaatcaatttttacgttataaaagtggtatcgcaaaacaatcaaagtaattctatataaatctggaatcacacagtatgattccacataactagttgtgtggatgttatgaaggtcacatacctatatcactgctttaaagctaaccgattgttacaaaggtttcaacttgtaagtacgatgagattaatttggggtggctatcaagtatcaaccaccacgcacgtgcatcaaccgttaggagaccacgtgatcgagctcggaagctgagcctaaaccaaaacaggagcacgagcgtgttaggctaggctgctaatgttagctgggtaataatacactccctctccattcatcagtataaaattataaatatatttttagtcaccttaagaagatcgcattaaacattctccttttcgaacttccctctctcccggagtctccgatcatcacctctatctatcctaagatggatcc

SEQ ID NO: 17 - DNA, 1238 bp, Artificial Sequence
  synthetic R. rugosa F3H 5′, 1.2 kb fragment containing Rosa rugosa flavonoid 3′-hydrolase (F3′H) gene promoter region
aagcttctagttagacaaaaagctactcatatttcttacatacttgttatatgtttgtgacggtttgtcttccccttacccaatccctggctttggtttttacgtgatctccaaatactgggtacatcgccataaggagcatttagtatattaggtgatcgtgacatatatctattcggacatttattgatatgaacagagaagagaagaatggtttctatggtatttcattcctattcagataaaatattaacagctctaagagagttgaattaatttctgttaaaaaagtgatcaggtgacgagattttagtaatgaggcataattagtagggggaaatgatcatttatcctgaggcagaattgggacatgttcatccgggacggctgatctactccccacctccataatgagtatttagtatattaggccgtgacatttatcgagtcggccctcataaaaagtttgtgactataccattttggctcaagtagttatgtacttatgttgtggtggtttcatatcacacgatgcatgtttcacaaattaataaaatttcacatacatgaaacaccgtaatcgtatttatccgcattaactactattttcatttatttaaaaaccatctttctttcttctttttcttcttgttaaaaatagcaaaaagtgaaaagaagaggtttttttttttttttcaaacattttcactcttctacatacatcgtttcaataacattcttgaaaacactggcggacctatctaagggcaagtgagggctcatgcgtctactgagtttttgtttgtaatggagtaaattaatgatttactagcttgtatacaaaaaattaacctaaatattagccctcacatcatttttttttttttaatgacaatatatctctgtataaatcaaattctgaggaatcgtcactgcttaaaaatgatatccaatctagggtccatagaatgccaaatattcaagaagccaaaaaatgggggtttggtatagcttgaggctagtttcagtttgtctttgtgatgactgaaagaagagaagctccctcacgagcgctttgaggtagttgtaggcttgtagctgccaacccctttttatcggccattaaatccccaatatataccacccctcacccctcaccccacaccaaacatcatcttcttcatcgcatttttcacttgtcttccccaaaacagaacggaaatatcaagagagaattc

SEQ ID NO: 18 - DNA, 1102 bp, Artificial Sequence
  synthetic BPF3′5′H#40 5′, 2.0 kb fragment containing flavonoid 3′,5,-hydroxylase (F3′5′H) cDNA promoter region
aagcttgtgatcgacatctctctcccggctccaacttgacggggggtgttaaacgtatacaagattttctaatcttgtatatttgattttctaatatcttgtatatttgattttctattatcttgtatttgaacttttgtatttccttagtatcaggaaagttagttgtagatattattttatatttcaaatctgtatctaatacttgcctatataaaggccaactaatcaatgaaatgaacacatcaattttctcaatttctcattctctgttttcatatctattctctattttcacattttctgaaaagaaagatgcttgacatgatcagagacagttctttcttcttcatactttcgtactaaacttctcctggtccgcaactaatcttccatcattttcttgtgatcttcacttgaggatagtctctagaaaacggcacggtcacgctggataagtgtttagctagcctcgaagttgagttgcatgaattttgcgggtacgcaagtgacttgactcttatcttggacgtcttatatgctcgaccaaatgttggccaagtcgggatgctcgggttaagcctctcttaggtcaagtttatgagcgaacccctttctttgagggctctttatttgccaactcgtctgccattaaagttctattagagctctaatgctgtgtatgtggctaccgatcaccttcattctcagaggaatcctcttttcgaatttctggtactttgaaactagctgcttcaatttcagccactcgaattaaacactaaaacagaacattgagaggaacgggccctcttccaaatatagaaagaaacagataatgtcaaaagacacatcaactaggtcgagatacctgctcacatgcatcacatctaaccaactcgagtcggacgagaaatgagttcgtaactcgatgataataaggcaaaggtctaaaaccacattcggttggtggttgtgttcatggaccgatcacgtgccctaacctaacccccgcatccatccaccaacagctagtcctcgccgagtcccccaaagttcctatttatatcactaaagtccctttttctcaacatagggatcc

SEQ ID NO: 19 - DNA, 20 bp, Artificial Sequence
  synthetic amplification primer Hf1-5
tcaaaatatttacttcggcc

SEQ ID NO: 20 - DNA, 20 bp, Artificial Sequence
  synthetic amplification primer Hf1-3
tttcattcgacatcctttgg

SEQ ID NO: 21 - DNA, 20 bp, Artificial Sequence
  synthetic amplification primer CHSA+34
acgtgacaagtgtaagtatc

SEQ ID NO: 22 - DNA, 20 bp, Artificial Sequence
  synthetic amplification primer CHSA-782
gttttccaaatcttgacgtg

SEQ ID NO: 23 - DNA, 35 bp, Artificial Sequence
  synthetic nested 1st PCR amplification primer C1
gtacatattgtcgttagaacgcgtaatacgactca

SEQ ID NO: 24 - DNA, 24 bp, Artificial Sequence
  synthetic nested 1st PCR amplification reverse orientation primer BP40-i5
aggtgcatgatcggaccatacttc

SEQ ID NO: 25 - DNA, 35 bp, Artificial Sequence
  synthetic nested 2nd PCR amplification primer C2
cgttagaacgcgtaatacgactcactatagggaga

SEQ ID NO: 26 - DNA, 25 bp, Artificial Sequence
  synthetic nested 2nd PCR amplification reverse orientation primer BP-i7
gaccatacttcttagcgagtttggc

SEQ ID NO: 27 - DNA, 25 bp, Artificial Sequence
  synthetic PCR amplification primer BP40 pro-F
actcaaacaagcatctcgccatagg

SEQ ID NO: 28 - DNA, 25 bp, Artificial Sequence
  synthetic PCR amplification primer BP40 pro-HindIII-F
aagcttgtgatcgacatctctctcc

SEQ ID NO: 29 - DNA, 21 bp, Artificial Sequence
  synthetic PCR amplification primer BP40 pro-NheI-R
cgaggctagctaaacacttat

SEQ ID NO: 30 - DNA, 20 bp, Artificial Sequence
  synthetic PCR amplification primer BP40 pro-NheI-F
tttagctagcctcgaagttg

SEQ ID NO: 31 - DNA, 27 bp, Artificial Sequence
  synthetic PCR amplification primer BP40 pro-BamHI-R
ggatccctatgttgagaaaaagggact

SEQ ID NO: 32 - DNA, 24 bp, Artificial Sequence
  synthetic PCR amplification primer DFRproHindIIIF for Fragment C
taataagcttacagtgtaattatc

SEQ ID NO: 33 - DNA, 23 bp, Artificial Sequence
  synthetic PCR amplification primer DFRproNheIR for Fragment C
ttatgctagcgtgtcaagaccac

SEQ ID NO: 34 - DNA, 22 bp, Artificial Sequence
  synthetic second PCR amplification primer DFRproNheIF for Fragment D
acacgctagcataagtctgttg

SEQ ID NO: 35 - DNA, 20 bp, Artificial Sequence
  synthetic second PCR amplification primer DFRproBamHI-R for Fragment D
gcttggggatccatcttagg

SEQ ID NO: 36 - DNA, 25 bp, Artificial Sequence
  synthetic PCR amplification primer RrF3H-F
aagcttctagttagacaaaaagcta

SEQ ID NO: 37 - DNA, 25 bp, Artificial Sequence
  synthetic PCR amplification primer RrF3H
ggatcctctcttgatatttccgttc

SEQ ID NO: 38 - DNA, 36 bp, Artificial Sequence
  synthetic PCR amplification forward primer DFRint F
gcatccatggagatcttcgtgatcctggtatgtttg

SEQ ID NO: 39 - DNA, 31 bp, Artificial Sequence
  synthetic PCR amplification reverse primer DFRint R
gcattctagacttcttgttctctacaaaatc

SEQ ID NO: 40 - DNA, 20 bp, Artificial Sequence
  synthetic PCR amplification primer Chrys Red F
cctcatcctcgggtcagtac

SEQ ID NO: 41 - DNA, 21 bp, Artificial Sequence
  synthetic PCR amplification primer Chrys Red R
gcaactggaccattctcaagc

SEQ ID NO: 42 - DNA, 30 bp, Artificial Sequence
  synthetic PCR amplification primer 3369Xba.F
gcatctctagaacaccatggcctatagtcg

SEQ ID NO: 43 - DNA, 30 bp, Artificial Sequence
  synthetic PCR amplification primer 3369Xba.R
gcatctctagattcaagaggatccgcccac

SEQ ID NO: 44 - DNA, 1660 bp, Artificial Sequence
  synthetic chrysF3′H.nt, full-length Chrysanthemum flavonoid 3′-hydrolase (F3′H) cDNA clone
aaaatgaccattttagctttcgtattttacgccctcatcctcgggtcagtactctatgtatttcttaacttaagttcacgtaaatccgccagactcccacccgggccaacaccatggcctatagtcgggaacttaccacaccttggcccaatcccacaccacgcactcgcggccttagccaagaagtacgggccattgatgcacctgcggctcgggtgtgtggacgtggttgtggccgcgtctgcttccgtagctgcacagtttttaaaagttcacgacgcaaattttgctagtaggccgccaaattctggcgcgaaacatgtggcgtataattatcaggatcttgtgtttgcaccttatggtccaaggtggcgtttgttaaggaagatttgttcggtccatttgttttctgctaaagcacttgatgattttcgtcatgttcgacaggaggaggtagcagtcctaacccgcgtactactgagtgctggaaactcaccggtacagcttggccaactacttaacgtgtgtgccacaaacgccttagcacgggtaatgttaggtaggagagttttcggagacggaattgacaggtcagccaatgagttcaaagatatggtagtagagttaatggtattagcaggagaatttaaccttggtgactttattcctgtacttgacctattcgacctacaaggcattactaaaaaaatgaagaagcttcatgttcggttcgattcatttcttagtaagatcgttgaggagcataaaacggcacctggtgggttgggtcatactgatttgctgagcacgttgatttcacttaaagatgatgctgatattgaaggtgggaagcttacagatactgaaatcaaagctttgcttctgaatttattcgctgcgggaacagacacatcctctagtacagtagaatgggcaatagccgaactcattcgtcatccacaaatattaaaacaagcccgagaagagatagacgctgtagttggtcaagaccggcttgtaacagaattggacttgagccaactaacatacctccaggctcttgtgaaagaggtgtttaggctccacccttcaacgccactctccttaccaagaatatcatccgagagttgtgaggtcgatgggtattatatccctaagggatccacactcctcgttaacgtgtgggccattgcgcgagacccaaaaatgtgggcggatcctcttgaatttaggccttctcggtttttacccgggggagaaaagcccggtgctgatgttaggggaaatgattttgaagttataccatttggggcaggacgaaggatttgtgcgggtatgagcctaggcttgagaatggtccagttgctcattgcaacattggtccaaacttttgattgggaactggctaacgggttagagccggagatgctcaacatggaagaagcgtatggattgacccttcaacgggctgcacccttgatggttcacccgaagccgaggttagctccccacgtatatgaaagtatttaaggactagtttctcttttgcctttttgtttcgcaaaggttaatgaataaacgatttcatgactcagatagttatgtaaacaattgtgtttgctgtttatatatttatctatttttctagaacaaaaaaaaa

SEQ ID NO: 45 - protein, 508 aa, Artificial Sequence
  synthetic chrysF3′H.nt, full-length Chrysanthemum flavonoid 3′-hydrolase (F3′H) cDNA clone deduced amino acid sequence
MTILAFVFYALILGSVLYVFLNLSSRKSARLPPGPTPWPIVGNLPHLGPIPHHALAALAKKYGPLMHLRLGCVDVVVAASASVAAQFLKVHDANFASRPPNSGAKHVAYNYQDLVFAPYGPRWRLLRKICSVHLFSAKALDDFRHVRQEEVAVLTRVLLSAGNSPVQLGQLLNVCATNALARVMLGRRVFGDGIDRSANEFKDMVVELMVLAGEFNLGDFIPVLDLFDLQGITKKMKKLHVRFDSFLSKIVEEHKTAPGGLGHTDLLSTLISLKDDADIEGGKLTDTEIKALLLNLFAAGTDTSSSTVEWAIAELIRHPQILKQAREEIDAVVGQDRLVTELDLSQLTYLQALVKEVFRLHPSTPLSLPRISSESCEVDGYYIPKGSTLLVNVWAIARDPKMWADPLEFRPSRFLPGGEKPGADVRGNDFEVIPFGAGRRICAGMSLGLRMVQLLIATLVQTFDWELANGLEPEMLNMEEAYGLTLQRAAPLMVHPKPRLAPHVYESI